SEQUENCE LISTING (18 sequences)

SEQ ID NO: 1 - protein, 265 aa, synthetic construct
MPFYDSRPPEGWPKGSINDMDYPLLGSICAVCCVFVAGSGIWMLYRLDLGMGYSCKPYKSGRAPEVNSLSGIICLLCGTMYAAKSFDFFDGGGTPFSLNWYWYLDYVFTCPLLILDFAFTLDLPHKIRYFFAVFLTLWCGVAAFVTPSAYRFAYYALGCCWFTPFALSLMRHVKERYLVYPPKCQRWLFWACVIFFGFWPMFPILFIFSWLGTGHISQQAFYIIHAFLDLTCKSIFGILMTVFRLELEEHTEVQGLPLNEPETLS

SEQ ID NO: 2 - protein, 265 aa, synthetic construct
MPFYDSRPPEGWPRGSVNDMDYPLLGSICAISAIAIAGSGIWMLYRLDLGMGYSCKPYKSGRAPEVNSISGIVCLLCGTMYAAKSFDFFDGGGTPFSLNWYWYLDYVFTCPLLIVDFAFTLDIPQKLRYSIAVFVALWCAVAAFATPSAFRFAYYALGCCWFIPLSLFLIRDVKKRYQVYPPKCQRLLFWACVVFFGFWPLFPLLFIFSWQGSGHISRQAYYIIHAFLDLVCKSIFGFLMTFFRLELEEHTEVQGLPLKEPKVMD

SEQ ID NO: 3 - DNA, 795 bp, synthetic construct
  source 1..795: mol_type = other DNA
atgcctttctacgacagccggccccctgagggatggcctaagggctccatcaacgacatggattatccactgctgggctctatttgtgccgtgtgctgcgtgttcgtggcaggcagcggcatctggatgctgtacaggctggatctgggcatgggctactcttgtaagccatataaaagcggccgggcacctgaggtgaacagcctgtccggcatcatctgcctgctgtgcggcacaatgtacgccgccaagtccttcgacttctttgatggcggcggaacccccttttctctgaattggtactggtatctggactacgtgttcacctgccccctgctgatcctggacttcgcctttaccctggatctgcctcacaagatccggtacttctttgccgtgtttctgaccctgtggtgtggagtggcagccttcgtgacaccaagcgcctatagatttgcctactatgccctgggctgctgttggttcacaccctttgccctgtccctgatgcggcacgtgaaggagagatacctggtgtatccacccaagtgccagaggtggctgttctgggcctgcgtgatcttctttggcttctggcctatgtttccaatcctgttcatcttttcctggctgggcaccggccacatctctcagcaggccttctacatcatccacgcctttctggatctgacctgcaagtctatcttcggcatcctgatgacagtgtttcgcctggagctggaggagcacacagaggtgcagggactgcccctgaatgagcctgagacactgtcc

SEQ ID NO: 4 - DNA, 798 bp, synthetic construct
  source 1..798: mol_type = other DNA
atgcccttctatgattccaggccccccgagggctggcccaagggcagcatcaacgatatggactaccccctgctgggctccatttgcgctgtgtgctgtgtcttcgtggctggctccggtatctggatgctgtacaggctggacctgggcatgggctacagctgtaaaccttataaatccggcagggcccctgaggtgaacagcctgagcggcattatctgcctgctgtgcggcaccatgtacgctgcaaagtccttcgacttttttgacggcgggggaactcccttttctctgaactggtattggtacctggactatgtgtttacctgccctctgctgattctggacttcgccttcacgctggacctgccccataagatcaggtacttcttcgccgtgttcctgacactgtggtgcggcgtggctgccttcgtgaccccatctgcttaccgctttgcttactacgccctgggctgttgctggttcacccccttcgccctgtccctgatgcgccacgtgaaggaacggtacctggtgtaccctcccaagtgccagcggtggctgttctgggcctgcgtgatcttcttcggcttttggcctatgttccccattctgttcatctttagctggctcggcaccggccacattagccagcaggccttctacattatccacgcttttctggacctgacatgcaaatccatcttcggcatcctgatgaccgttttccggctggagctggaggagcacaccgaggtgcagggactgcccctgaatgaaccagagaccctgtcctga

SEQ ID NO: 5 - DNA, 795 bp, synthetic construct
  source 1..795: mol_type = other DNA
atgcctttctacgacagcaggccccctgagggatggcctcgcggctccgtgaacgacatggattatccactgctgggctctatctgcgccatcagcgccatcgcaatcgcaggctccggcatctggatgctgtaccggctggatctgggcatgggctactcctgtaagccatataagtctggcagagcccccgaggtgaacagcatctccggcatcgtgtgcctgctgtgcggcaccatgtacgccgccaagtctttcgacttctttgatggcggcggcacaccctttagcctgaattggtactggtatctggactacgtgttcacctgccccctgctgatcgtggacttcgccttcaccctggacatccctcagaagctgaggtattccatcgccgtgttcgtggccctgtggtgtgcagtggcagcctttgccaccccatctgccttccgctttgcctactatgccctgggctgctgttggttcatccccctgagcctgtttctgatccgggatgtgaagaagagataccaggtgtatccacccaagtgccagagactgctgttctgggcctgcgtggtgttcttcggcttctggcctctgtttccactgctgttcatcttttcttggcagggcagcggacacatctcccggcaggcctactatatcatccacgcctttctggacctggtgtgcaagtctatcttcggctttctgatgaccttctttcggctggagctggaggagcacacagaggtgcagggcctgcccctgaaggagcctaaagtgatggat

SEQ ID NO: 6 - DNA, 798 bp, synthetic construct
  source 1..798: mol_type = other DNA
atgcccttctatgattccaggccccccgagggctggcccagaggcagcgtgaacgatatggactaccccctgctgggctccatttgcgctatcagcgctatcgccatcgctggctccggtatctggatgctgtacaggctggacctgggcatgggctacagctgtaaaccttataaatccggcagggcccctgaggtgaacagcatcagcggcattgtgtgcctgctgtgcggcaccatgtacgctgcaaagtccttcgacttttttgacggcgggggaactcccttttctctgaactggtattggtacctggactatgtgtttacctgccctctgctgattgtggacttcgccttcacgctggacatcccccagaagctgaggtactccatcgccgtgttcgtggcactgtggtgcgccgtggctgccttcgccaccccatctgctttccgctttgcttactacgccctgggctgttgctggttcatccccctgtccctgttcctgattcgcgatgtgaagaaacggtaccaggtgtaccctcccaagtgccagcggctgctgttctgggcctgcgtggtgttcttcggcttttggcctctgttccccctgctgttcatctttagctggcaaggctctggccacattagcagacaggcctactacattatccacgcttttctggacctggtgtgcaaatccatcttcggcttcctgatgaccttcttccggctggagctggaggagcacaccgaggtgcagggactgcccctgaaggaaccaaaggtcatggactga

SEQ ID NO: 7 - protein, 13 aa, synthetic construct
GRKKRRQRRRPPQ

SEQ ID NO: 8 - protein, 16 aa, synthetic construct
RQIKIWFQNRRMKWKK

SEQ ID NO: 9 - protein, 26 aa, synthetic construct
GIGAVLKVLTTGLPALISWIKRKRQQ

SEQ ID NO: 10 - protein, 10 aa, synthetic construct
TKIESLKEHG

SEQ ID NO: 11 - protein, 10 aa, synthetic construct
TQIENLKEKG

SEQ ID NO: 12 - protein, 26 aa, synthetic construct
AALEALAEALEALAEALEALAEAAAA

SEQ ID NO: 13 - protein, 25 aa, synthetic construct
GLFEAIEGFIENGWEGMIEGWYGCG

SEQ ID NO: 14 - protein, 265 aa, synthetic construct; identical to SEQ ID NO: 1

SEQ ID NO: 15 - protein, 265 aa, synthetic construct; identical to SEQ ID NO: 2

SEQ ID NO: 16 - protein, 300 aa, Guillardia theta
MVASSAVITANWISFLAISASFIILLVISLRYKGPGGTESFYNGFKEQNMLTVFINLWCALAYFAKVLQSHSNDNGFAPLTVIPYVDYCTTCPLLTLDLLWCLDAPYKISSAVLVFTCLVIAVACSLAVAPFSYCWFAMGMVLFTFTYVFILSIVRQRLDFFTLCARDSNAKQSLKHLKTAVFIYFGIWLLFPLLWLLSYRAANVISNDINHIFHCILDVIAKSVYGFALLYFKMYFDKKLIESGIDEDDFAKFSKVVTTHRSEEKQKRKPAVSQSPKMYYDEAAYQDGEVESNLQSKIR

SEQ ID NO: 17 - protein, 331 aa, synthetic construct
MVSEGYGDKYLEGSMRGWYTPCTFGTPAIMAHWVTFLFLFVCTMYLARDSWTARGPGGRERWYAGYHEDYNISLYVNLFAMIAYFGKIVADSVGHNYNNVGPLVIGLGNYRYADYMLTCPLLAYDLMSQVRAPFKITGGVLIFCVLLTGTATNYYPGDEMRAGSMAWFIFGCFLYFVAYYLFYTVVKIQYTRLLALAASTEAKKAFMPLKLALYTFFSIWIVFPIVWLLGHQGLNILSNDAQECLHCACDLTAKSFYGFALAKYRKYFDKKMFDMLTELGIDGEEGLEHLEQDLKNQDIGDGKADIKRMSTTRGPGLSVISVNKHHPAFLA

SEQ ID NO: 18 - protein, 309 aa, synthetic construct
MAHAPGTDQMFYVGTMDGWYLDTKLNSVAIGAHWSCFIVLTITTFYLGYESWTSRGPSKRTSFYAGYQEEQNLALFVNFFAMLSYFGKIVADTLGHNFGDVGPFIIGFGNYRYADYMLTCPMLVYDLLYQLRAPYRVSCSAIIFAILMSGVLAEFYAEGDPRLRNGAYAWYGFGCFWFIFAYSIVMSIVAKQYSRLAQLAQDTGAEHSLHVLKFAVFTFSMLWILFPLVWAICPRGFGWIDDNWTEVAHCVCDIVAKSCYGFALARFRKTYDEELFRLLEQLGHDEDEFQKLELDMRLSSNGERLRRLS

(19) United States
(12) Patent Application Publication
(10) Pub. No.: US 2024/0101637 A1
(43) Pub. Date: Mar. 28, 2024

(54) COMPOSITONS AND METHODS FOR USE OF KALIUM CHANNEL RHODOPSINS

(71) Applicants: THE BOARD OF REGENTS OF THE UNIVERSITY OF TEXAS SYSTEM, Austin, TX (US); BAYLOR COLLEGE OF MEDICINE, Houston, TX (US)

(72) Inventors: John Lee SPUDICH, Houston, TX (US); Elena G. GOVORUNOVA, Houston, TX (US); Oleg A. SINESHCHEKOV, Houston, TX (US); Mingshan XUE, Houston, TX (US)

(73) Assignees: THE BOARD OF REGENTS OF THE UNIVERSITY OF TEXAS SYSTEM, Austin, TX (US); BAYLOR COLLEGE OF MEDICINE, Houston, TX (US)

(21) Appl. No.: 18/465,333

(22) Filed: Sep. 12, 2023

Related U.S. Application Data
(60) Provisional application No. 63/375,354, filed on Sep. 12, 2022.

(51) Int. Cl. C07K 14/705; A61K 48/00; A61P 27/02

(52) U.S. Cl. CPC C07K 14/705; A61K 48/0058; A61P 27/02

(57) ABSTRACT

Methods and compositions used to identify and characterize novel rhodopsin domains, which are kalium (potassium)-conducting channelrhodopsins. The rhodopsin domain of these kalium (potassium)-conducting channelrhodopsins have been cloned, optimized and expressed in mammalian systems and thus may be used in, among others, optogenetic applications and as therapeutic agents for electrically active cell mediated disorders.

### PRIORITY CLAIM

This application claims benefit of priority to U.S. Provisional Application Ser. No. 63/375,354, filed Sep. 12, 2022, the entire contents of which are hereby incorporated by reference.

### STATEMENT REGARDING FEDERALLY FUNDED RESEARCH

This invention was made with U.S. Government support under Grant Nos. R35GM140838 and U01NS118288, awarded by the National Institutes of Health. The government has certain rights in the invention.

### INCORPORATION OF SEQUENCE LISTING

This application contains a Sequence Listing XML, which has been submitted electronically and is hereby incorporated by reference in its entirety. Said XML Sequence Listing, created on Sep. 10, 2022, is named UTSHP0392US.xml and is 21,575 bytes in size.

### BACKGROUND OF THE INVENTION

### 1. Field of the Invention

This disclosure generally relates to the fields of molecular biology and medicine. Methods and compositions that utilize channelrhodopsins, such kalium-conducting channelrhodopsins, are provided. In some aspects, channelrhodopsins can be used for optogenetic applications or as therapeutic agents.

### 2. Description of Related Art

Optogenetics (Deisseroth, 2011; Deisseroth, 2015), refers to using optical methods for probing and controlling genetically targeted neurons within intact neural circuits. Optogenetics involves the introduction of light-activated channels and enzymes that allow manipulation of neural activity with millisecond precision while maintaining cell-type resolution through the use of specific targeting mechanisms. Because the brain is a high-speed system, millisecond-scale temporal precision is central to the concept of optogenetics, which allows probing the causal role of specific action potential patterns in defined cells.

Light control of motility behavior (phototaxis and photophobic responses) in green flagellate algae is mediated by sensory rhodopsins homologous to phototaxis receptors and light-driven ion transporters in prokaryotic organisms. In the phototaxis process, excitation of the algal sensory rhodopsins leads to generation of transmembrane photoreceptor currents. When expressed in animal cells, the algal phototaxis receptors function as light-gated cation channels, which has earned them the name “channelrhodopsins”. Channelrhodopsins have become useful molecular tools for light control of cellular activity.

Originally, the source of these light-activated channels and enzymes were several microbial opsins, including Channelrhodopsin-2 (ChR2) a single-component light-activated cation channel from algae, which allowed millisecond-scale temporal control in mammals, required only one gene to be expressed in order to work, and responded to visible-spectrum light with a chromophore (retinal) that was already present and supplied to ChR2 by the mammalian brain tissue. The experimental utility of ChR2 was quickly proven in a variety of animal models ranging from behaving mammals to classical model organisms such as flies, worms, and zebrafish, and hundreds of groups have employed ChR2 and related microbial proteins to study neural circuits. Currently, several members of this family have been recruited as molecular tools for optogenetics, i.e. regulation of cellular activity with light. Phototaxis receptors from green (chlorophyte) flagellate algae (6), best known as channelrhodopsins (ChRs) owing to their function as light-gated cation channels are widely used to depolarize genetically targeted populations of excitable cells.

Hyperpolarizing rhodopsin ion pumps have been employed to suppress neuron firing, but they transport only a single charge per captured photon and therefore have limited capacity. Recently, ChRs were engineered to conduct Cl—, but these optogenetic tools still retain some cation conductance and could be made highly light-sensitive only at the expense of greatly slowing the channel kinetics with additional mutations (Wietek et al., 2014; Berndt et al., 2014). Ideal for optogenetic hyperpolarization would be natural light-gated potassium channels optimized by evolution to be highly conductive and selective.

Described herein are modified and optimized rhodopsin domains derived from a newly identified class of channelrhodopsins, Kalium Channel Rhodopsins (KCRs), which are light-gated potassium (kalium) channels that provide membrane hyperpolarization and neuronal silencing through light-gated potassium conduction.

### SUMMARY OF THE INVENTION

Thus, in accordance with the present disclosure, there is provided a recombinant nucleic acid operatively linked to a heterologous promoter sequence, said recombinant nucleic acid comprising a kalium-conducting rhodopsin domain encoded by a nucleic acid sequence that encodes a polypeptide sequence that is at least about 90% identical to SEQ ID NO: 1 or SEQ ID NO: 2. In another embodiment, there is provided a recombinant nucleic acid operatively linked to a heterologous promoter sequence, said recombinant nucleic acid comprising a kalium-conducting rhodopsin domain encoded by a nucleic acid sequence that is at least about 90% identical to a sequence selected from the group consisting of SEQ ID NO: 3, SEQ ID NO: 4, SEQ ID NO: 5 and SEQ ID NO: 6. The recombinant nucleic acid of at least about 90% identity to a sequence selected from the group consisting of SEQ ID NO: 3, SEQ ID NO: 4, SEQ ID NO: 5 or SEQ ID NO: 6, or the complement thereof, may hybridize to filter-bound SEQ ID NO: 3, SEQ ID NO: 4, SEQ ID NO: 5 or SEQ ID NO: 6 in 0.5 M NaHPO₄, 7% sodium dodecyl sulfate, 1 mM EDTA at 65° C., and washing in 0.2×SSC/0.1% SDS at 42° C. Also provided is an expression vector comprising these recombinant nucleic acids.

In another embodiment, there is provided a recombinant host cell comprising a recombinant nucleic acid as defined herein, or the expression vector comprising the recombinant nucleic acid. The recombinant host cell of claim 4, wherein said host cell is an isolated human cell, a non-human mammalian cell, a bacterial cell, a yeast cell an insect cell, a plant cell, an isolated neuronal cell, or an isolated electrically active cell.

Also provided is a method of membrane hyperpolarization of a cell in a subject suffering from a neuron mediated disorder, said method comprising delivering to the cell of said subject an expression vector comprising a polynucleotide sequence at least about 90% identical to a sequence selected from the group consisting of SEQ ID NO: 3, SEQ ID NO: 4, SEQ ID NO: 5 or SEQ ID NO: 6, encoding a kalium-conducting rhodopsin domain expressible in said cell, wherein the expressed rhodopsin silences a signal from said neuron.

In yet another embodiment, there is provided a method of membrane hyperpolarization of a cell in a subject suffering from a neuron mediated disorder, said method comprising delivering to the cell of said subject an expression vector comprising a polynucleotide sequence that encodes the polypeptide sequence that is at least about 90% identical to SEQ ID NO: 1 or SEQ ID NO: 2, encoding a kalium-conducting rhodopsin domain expressible in said cell, wherein the expressed rhodopsin silences a signal from said neuron.

Further provided is a method of neuronal silencing in a subject suffering from a neuron mediated disorder, said method comprising delivering to a target neuron of said subject an expression vector comprising a polynucleotide that encodes a nucleic acid sequence at least about 90% identical to a sequence selected from the group consisting of SEQ ID NO: 3, SEQ ID NO: 4, SEQ ID NO: 5 or SEQ ID NO: 6, encoding a rhodopsin domain of a kalium-conducting channelrhodopsin expressible in said target neuron, wherein the expressed rhodopsin results in silencing of the signal from the target neuron.

Even further provided is a method of restoring photosensitivity to a retina of a subject suffering from vision loss or blindness, said method comprising delivering to the retina of said subject an expression vector comprising a polynucleotide that encodes a nucleic acid sequence at least about 90% identical to a sequence selected from the group consisting of SEQ ID NO: 3, SEQ ID NO: 4, SEQ ID NO: 5 or SEQ ID NO: 6, encoding a rhodopsin domain of a kalium-conducting channelrhodopsin expressible in a retinal neuron, wherein the expressed rhodopsin produces a high level of membrane potential in said retinal neuron.

These methods may comprise delivering to the cell of said subject an expression vector that encodes a rhodopsin domain; said vector comprising an open reading frame encoding a nucleic acid sequence at least about 90% identical to a sequence selected from the group consisting of SEQ ID NO: 3, SEQ ID NO: 4, SEQ ID NO: 5 or SEQ ID NO: 6, operatively linked to a promoter sequence, wherein the expressed rhodopsin produces a high level of membrane potential in said cell.

These methods may comprise delivering to the cell of said subject an expression vector that encodes a rhodopsin domain; said vector comprising an open reading frame encoding the rhodopsin domain of a kalium-conducting channelrhodopsin selected from SEQ ID NO: 3, SEQ ID NO: 4, SEQ ID NO: 5 or SEQ ID NO: 6, operatively linked to a promoter sequence, wherein the expressed rhodopsin produces a high level of membrane potential in said cell.

In each of the foregoing methods, the recombinant nucleic acid of at least about 90% identity to a sequence selected from the group consisting of SEQ ID NO: 3, SEQ ID NO: 4, SEQ ID NO: 5 or SEQ ID NO: 6, or the complement thereof, may hybridize to filter-bound SEQ ID NO: 3, SEQ ID NO: 4, SEQ ID NO: 5 or SEQ ID NO: 6 in 0.5 M NaHPO₄, 7% sodium dodecyl sulfate, 1 mM EDTA at 65° C., and washing in 0.2×SSC/0.1% SDS at 42° C. The subject may be mammalian or human. Delivery may comprise a pharmaceutically acceptable carrying agent.

Also provided is an isolated nucleic acid comprising a nucleic acid sequence that encodes a nucleic acid sequence at least about 90% identical to a sequence selected from the group consisting of SEQ ID NO: 3, SEQ ID NO: 4, SEQ ID NO: 5 or SEQ ID NO: 6. Or an expression vector encoding the same.

Still further provided are:
a method of treating a disorder in an electrically active cell in a subject suffering from a disorder that involves electrically active cells, said method comprising delivering to the cell of said subject an expression vector comprising a polynucleotide having a nucleic acid sequence at least about 90% identical to a sequence selected from the group consisting of SEQ ID NO: 3, SEQ ID NO: 4, SEQ ID NO: 5 or SEQ ID NO: 6, expressible in said cell, wherein the expressed rhodopsin silences the signal from said electrically active cell;
a method of treating a disorder in an electrically active cell in a subject suffering from a disorder that involves electrically active cells, said method comprising delivering to said subject a transgenic cell comprising an expression vector comprising a polynucleotide that encodes a nucleic acid sequence at least about 90% identical to a sequence selected from the group consisting of SEQ ID NO: 3, SEQ ID NO: 4, SEQ ID NO: 5 or SEQ ID NO: 6 expressible in said transgenic cell, wherein the expression silences the signal from an electrically active cell; or
a method of silencing an electrically active cell in a subject suffering from an electrically active cell mediated disorder, said method comprising delivering to a target neuron of said subject an expression vector comprising a polynucleotide that encodes a nucleic acid sequence at least about 90% identical to a sequence selected from the group consisting of SEQ ID NO: 3, SEQ ID NO: 4, SEQ ID NO: 5 or SEQ ID NO: 6, encoding a rhodopsin domain of an kalium-conducting channelrhodopsin expressible in said target neuron, wherein the expressed rhodopsin results in silencing of the signal from the electrically active cell.

In still yet a further embodiment, there is provided an isolated nucleic acid molecule comprising a sequence encoding a kalium-conducting channelrhodopsin having a sequence at least about 90% identical to a sequence according to SEQ ID NO: 3, SEQ ID NO: 4, SEQ ID NO: 5 or SEQ ID NO: 6. The sequence may have a sequence at least about 95% identical to a sequence according to SEQ ID NO: 3, SEQ ID NO: 4, SEQ ID NO: 5 or SEQ ID NO: 6. The nucleic acid may be a DNA, an RNA; or an mRNA. Other embodiments comprising an expression vector comprising such nucleic acid molecule, or a recombinant host cell comprising such nucleic acid molecule or expression vector. Such recombinant host cell may be is an isolated human cell, a non-human mammalian cell, a bacterial cell, a yeast cell, an insect cell, a plant cell, an isolated neuronal cell, or an isolated electrically active cell.

In even yet a further embodiment, there is provided a method of treating a subject suffering from a disorder that involves electrically active cells comprising expressing in the subject an effective amount of a kalium-conducting channelrhodopsin at the site of the electrically active cells. The subject may be suffering from neuropathic pain, and/or have an amputated limb, diabetes, multiple sclerosis and/or has undergone a surgery. The method may comprise expressing an effective amount of a kalium-conducting channelrhodopsin at the site of the pain. Expressing may comprise administering a kalium-conducting channelrhodopsin to the subject. The kalium-conducting channelrhodopsin may further comprise a cell-penetrating peptide (CPP) sequence or a cellular receptor-binding sequence. Expressing may comprise administering a vector encoding a kalium-conducting channelrhodopsin to the subject, such as an RNA vector or a DNA vector. The sequence encoding the kalium-conducting channelrhodopsin may be operably linked to a heterologous promoter, such as an inducible or a repressible promoter. The promoter may also be a tissue or cell type specific promoter, such as a neuronal cell specific promoter. The vector may be a plasmid, a viral vector or an episomal vector, and may optionally further comprises an inducible expression cassette for a suicide gene. The kalium-conducting rhodopsin domain may comprise a nucleic acid sequence at least about 90% identical to a sequence of SEQ ID NO: 3, SEQ ID NO: 4, SEQ ID NO: 5 or SEQ ID NO: 6, or the kalium-conducting rhodopsin domain may be encoded by a sequence at about 90% identical to sequence according to SEQ ID NO: 3, SEQ ID NO: 4, SEQ ID NO: 5 or SEQ ID NO: 6.

The presently disclosed methods and compositions are based, in part, on the discovery and identification of a novel class of channelrhodopsins, Kalium (potassium) Channel Rhodopsins (KCRs). Light-gated rhodopsin cation channels derived from chlorophyte algae have transformed neuroscience research through their use as membrane-depolarizing optogenetic tools for targeted photoactivation of neuron firing. Photosuppression of neuronal action potentials has been limited by the lack of equally efficient tools for membrane hyperpolarization. Described herein are modified and optimized rhodopsin domains derived from a newly identified class of channelrhodopsins, Kalium Channel Rhodopsins (ACRs), light-gated potassium (kalium) channels that provide efficient membrane hyperpolarization and rapid neuronal silencing through light-gated chloride conduction.

Sequences encoding 7TM domains of new ACR are provided. These ACRs provide new membrane-hyperpolarizing tools for use in establishing a high level of membrane potential for use as optogenetic tools for neuronal silencing of excited cells for among others, neuronal or neurologic disorders, such as but not limited to Parkinson's disease and epilepsy, as well as for cardiac disorders (as described for example by the inventors in Govorunova et al., 2016).

In some embodiments herein is disclosed a recombinant nucleic acid operatively linked to a heterologous promoter sequence, said recombinant nucleic acid comprising: a sequence that encodes a polypeptide with at least 85%, 90%, 91%, 92%, 93%, 94%, 95%, 96%, 97%, 98% or 99% identical to a sequence selected from the group consisting of SEQ ID NO: 1 or SEQ ID NO: 2; or a sequence that encodes a polypeptide comprising at least 225, 230, 235, 240, 255, 260, 265, 270, 275, 280, 285, 290, 295, 300, 305, 310, 315 or 320 contiguous amino acids from SEQ ID SEQ ID NO: 1 or SEQ ID NO: 2 or a sequence that hybridizes to the nucleotide sequence of SEQ ID NO: 3, SEQ ID NO: 4, SEQ ID NO: 5, SEQ ID NO: 6, or the complement thereof. In another embodiment, the recombinant nucleic acid comprises an expression vector. In further embodiment, the recombinant nucleic acid that hybridized to the nucleotide sequence of SEQ ID NO: 3, SEQ ID NO: 4, SEQ ID NO: 5, SEQ ID NO: 6, or the complement thereof, further comprises hybridization to filter-bound DNA in 0.5 M NaHPO₄, 7% sodium dodecyl sulfate, 1 mM EDTA at 65° C., and washing in 0.2×SSC/0.1% SDS at 42° C. In a further embodiment, there is provided a recombinant host cell comprising a recombinant nucleic acid operatively linked to a heterologous promoter sequence, said recombinant nucleic acid comprising: a sequence that encodes a polypeptide with at least 85%, 90%, 91%, 92%, 93%, 94%, 95%, 96%, 97%, 98% or 99% identity to an amino acid sequence selected from SEQ ID NO: 1 or SEQ ID NO: 2 or a sequence that encodes a polypeptide comprising at least 225, 230, 235, 240, 255, 260, 265, 270, 275, 280, 285, 290, 295, 300, 305, 310, 315 or 320 contiguous amino acids from SEQ ID SEQ ID NO: 1 or SEQ ID NO: 2 or a sequence that hybridizes to the nucleotide sequence of SEQ ID NO: 3, SEQ ID NO: 4, SEQ ID NO: 5, SEQ ID NO: 6, or the complement thereof.

In some embodiments, the host cell is an isolated human cell, a non-human mammalian cell, a bacterial cell, a yeast cell, an insect cell, or a plant cell.

In some embodiments, there is provided a method of restoring photosensitivity to a retina of a subject suffering from vision loss or blindness, said method comprising: delivering to the OFF-bipolar neurons of the retina of said subject an expression vector comprising a polynucleotide that encodes: an amino acid sequence at least about 85%, 90%, 91%, 92%, 93%, 94%, 95%, 96%, 97%, 98% or 99% identical to a sequence selected from the group consisting of SEQ ID NO: 1 or SEQ ID NO: 2 which encodes a humanized rhodopsin domain of an ACR expressible in a retinal neuron; and expressing said vector in said retinal neuron, wherein the expressed rhodopsin renders said retinal neuron photosensitive, thereby restoring photosensitivity to enable light-induced silencing of such neuron in said retina or a portion thereof. In a further embodiment, the subject is mammalian, and in a still further embodiment, the subject is human. In some aspects, the method comprises delivering to the retina of said subject an expression vector. In certain aspects, the delivery comprises a pharmaceutically acceptable carrying agent.

In some further embodiments, there is provided an isolated nucleic acid molecule comprising a sequence encoding a rhodopsin domain of an ACR having a sequence at least about 85%, 90%, 91%, 92%, 93%, 94%, 95%, 96%, 97%, 98% or 99% identical to a sequence according to SEQ ID NO: 3, SEQ ID NO: 4, SEQ ID NO: 5 or SEQ ID NO: 6, In certain aspects, the isolated nucleic acid molecule comprises a sequence that hybridized to the nucleotide sequence of one of SEQ ID NO: 3, SEQ ID NO: 4, SEQ ID NO: 5 or SEQ ID NO: 6, under stringent conditions comprising hybridization to filter-bound DNA in 0.5 M NaHPO₄, 7% sodium dodecyl sulfate, 1 mM EDTA at 65° C., and washing in 0.2×SSC/0.1% SDS at 42° C. and encodes an kalium-conducting channelrhodopsin. In some aspects, the nucleic acid is a DNA. In other aspects, the nucleic acid is an RNA (e.g., mRNA). In further embodiments, there is provided an expression vector comprising a nucleic acid molecule provided herein, such as a sequence at least about 90% identical to a sequence according to SEQ ID NO: 3, SEQ ID NO: 4, SEQ ID NO: 5, or SEQ ID NO: 6.

In an even further embodiment, there is provided a recombinant host cell comprising a nucleic acid provided herein (e.g., a sequence at least about 90% identical to a sequence according to SEQ ID NO: 3, SEQ ID NO: 4, SEQ ID NO: 5, or SEQ ID NO: 6). In some aspects, the host cell is an isolated human cell. In other aspects, the host cell is a non-human mammalian cell. In some aspects, the host cell is a bacterial cell. In certain aspects, the host cell is a yeast cell. In other aspects, the host cell is an insect cell. In some aspects, the host cell is a plant cell. In certain aspects, host cell is an isolated neuronal cell. In particular, the host cell is an isolated electrically active cell.

In another embodiment, there is provided a method of treating a subject suffering from a disorder that involves electrically active cells comprising expressing in the subject an effective amount of a sequence encoding a rhodopsin domain of an KCR kalium (potassium)-conducting channelrhodopsin at the site of the electrically active cells. In some aspects, the subject is suffering from neuropathic pain the method comprising expressing in the subject an effective amount of a kalium-conducting channelrhodopsin at the site of the pain. In certain aspects, the subject has an amputated limb, diabetes, multiple sclerosis or has undergone a surgery.

In certain aspects, expressing comprises administering a kalium-conducting channelrhodopsin to the subject. In some aspects, the kalium-conducting channelrhodopsin comprises an amino acid sequence at least about 85%, 90%, 91%, 92%, 93%, 94%, 95%, 96%, 97%, 98% or 99% identical to a sequence of SEQ ID NO:1 or SEQ ID NO: 2. In certain aspects, the kalium-conducting channelrhodopsin is encoded by a sequence about 90%, 91%, 92%, 93%, 94%, 95%, 96%, 97%, 98% or 99% identical to sequence according to SEQ ID NO: 3, SEQ ID NO: 4, SEQ ID NO: 5 or SEQ ID NO: 6. In some aspects, the kalium-conducting channelrhodopsin further comprises a cell-penetrating peptide (CPP) sequence or a cellular receptor-binding sequence. As used herein the terms “cell penetrating peptide” refers to segments of polypeptide sequence that allow a polypeptide to cross the cell membrane (e.g., the plasma membrane in the case a eukaryotic cell). Examples of CPP segments include, but are not limited to, segments derived from HIV Tat (e.g., GRKKRRQRRRPPQ (SEQ ID NO: 7)), herpes virus VP22, the Drosophila Antennapedia homeobox gene product, protegrin I, Penetratin (RQIKIWFQNRRMKWKK (SEQ ID NO: 8) or melittin (GIGAVLKVLTTGLPALISWIKRKRQQ (SEQ ID NO: 9)). In certain aspects the CPP comprises the T1 (TKIESLKEHG (SEQ ID NO: 10)), T2 (TQIENLKEKG (SEQ ID NO: 11)), 26 (AALEALAEALEALAEALEALAEAAAA (SEQ ID NO: 12)) or INF7 (GLFEAIEGFIENGWEGMIEGWYGCG (SEQ ID NO: 13)) CPP sequence.

In some aspects, expressing comprises administering a vector encoding an kalium-conducting channelrhodopsin to the subject encodes a rhodopsin domain of an KCR. In certain aspects, the vector is an RNA vector. In other aspects, the vector is a DNA vector. In some aspects, the vector is a plasmid, a viral vector or an episomal vector. In certain aspects, the vector further comprises an inducible expression cassette for a suicide gene.

In certain aspects, the sequence encoding the kalium-conducting channelrhodopsin encodes a rhodopsin domain of an KCR is operably linked to a heterologous promoter. In some aspects, the promoter is an inducible or a repressible promoter. In certain aspects, the promoter is a tissue or cell type specific promoter. In particular, the promoter is neuronal cell specific promoter.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Phylogenetic relations of KCRs with other known ChRs. The branches are colored to distinguish different ChR families. The leaves corresponding to the KCRs characterized in this study are shown as thick red lines. A full list of other ChR sequences used to create the tree can be found in (Govorunova et al., 2021). The gray circles show ultrafast bootstrap support values above 95%.

FIGS. 2A-I. KCR photocurrents evoked by continuous light and 2P excitation. FIG. 2a, The action spectra of photocurrents in the visible range (mean±sem, n=6 and 10 cells for HcKCR1 and HcKCR2, respectively) constructed from measurements of their initial slopes, as described in Methods. FIG. 2b, FIG. 2d, Photocurrent traces recorded from HcKCR1 in response to 1-s light pulses (540 nm, 6.3 mW mm-2) upon 20-mV voltage increments. FIG. 2c, FIG. 2f The IV curves (mean±sem, n=7 cells from 3 independent experiments) under indicated ionic conditions. FIG. 2e, Relative permeabilities (PX/PK); lines, mean±sem (n=7 cells from 3 independent experiments for Li+, Cs+, NMDG+, Mg2+ and Ca2+, 8 cells for Rb+ and 10 cells for Na+, each from 4 independent experiments); symbols, data from individual cells. FIG. 2g, Photocurrent traces recorded upon 2P excitation (HcKCR1, 1040 nm, 3.8 mW; HcKCR2, 1000 nm, 3.5 mW). FIG. 2h, Dependence of 2P photocurrents on the laser power (mean±sem, n=7 and 8 cells for HcKCR1 and HcKCR2, respectively; each from 3 independent experiments). FIG. 2i, Action spectra of photocurrents under 2P excitation (mean±sem, n=6 and 10 cells, each from 3 independent experiments, for HcKCR1 and HcKCR2, respectively). Source data are provided.

FIGS. 3a-c. Determination of HcKCR1 unitary conductance. FIG. 3a, Current noise in the dark (black) and under 540 nm, 60 μW mm-2 illumination (red) recorded from a HcKCR1-transfected HEK293 cell. RMS, root mean square. FIG. 3b, Power spectra of the noise in the dark (black) and under illumination (red). FIG. 3c, The difference (light minus dark) power spectrum (black) and its Lorenzian fit (red). γ, unitary conductance. For more detail see Methods.

FIGS. 4a-i. Photocurrents and photochemical conversion upon single quantum excitation. FIG. 4a, Photocurrent traces (thin solid lines) recorded from HcKCR1 in response to laser flashes upon 20-mV voltage increments superimposed with their multiexponential approximations (thick dashed lines). FIG. 4b, Voltage dependence of the three kinetic components of channel currents. FIG. 4c, Dependence of the channel current Vrev with 130 mM KCl in the bath and pipette on bath pH (mean±sem, n=3, 8, and 6 cells from 3 independent experiments for pH 5.4, 7.4, and 9.4, respectively). FIG. 4d, Time course of the peak current recovery (mean±sem, n=5 cells from 3 independent experiments). FIG. 4e, Transient absorbance changes (blue and black) and channel current (red). FIG. 4f, Photocurrent traces in the absence of permeant metal cations at bath pH 7.4. FIG. 4g, Voltage dependence of active current at different bath pH in the absence of permeant metal cations. FIG. 4h, Transient absorbance changes at 400 nm (blue), compared to active and passive (channel) currents (red and black, respectively). FIG. 4i, Photocurrent traces in the absence of permeant metal cations at bath pH 9.4. Source data are provided.

FIGS. 5a-i. Photoactivation of HcKCR1 in neurons generates robust photocurrents and efficiently suppresses neuronal firing. FIG. 5a, Fluorescent images of a cortical slice showing HcKCR1-EYFP and tdTomato expression in layer 2/3 neurons. Similar results were observed in 14 slices from 2 male and 2 female mice at the age of 3-4 weeks. Cortical layers were identified by DAPI staining. L, layer. FIG. 5b, Photocurrents traces of a HcKCR1-expressing neuron in response to a 1-s 565 nm light pulse (13.1 mW. mm-2) at holding voltages increased in 5-mV steps. FIG. 5c, The fast τ of photocurrent desensitization at the indicated voltages. P=0.2 by the two-tailed Wilcoxon test. FIG. 5d, Ratios of photocurrent at the end of illumination to the peak photocurrent. ***, P=0.0004 by the two-tailed paired t-test. FIG. 5e, FIG. 5f, IV curves of peak photocurrent (FIG. 5e) and photocurrent at the end of illumination (FIG. 5f) in individual neurons indicated by different colors. P<0.0001, R2>0.98 for all linear regressions. FIG. 5g, Reversal potentials calculated from the data in e and f. ****, P<0.0001 by the two-tailed paired t-test. FIG. 5h, Membrane voltage traces of a HcKCR1-expressing neuron in response to 0.2 (left) or 0.5 nA (right) current injections without (top) and with (bottom) 565 nm light pulses. FIG. 5i, The frequencies of action potentials evoked by different current injections with (magenta) and without (black) photoactivation. P>0.9999 for 0.1 nA and **, P=0.039 for 0.2-0.5 nA by the Multiple Wilcoxon matched-pairs signed rank test with Bonferroni-Dunn multiple corrections. Data in FIG. 5c, FIG. 5d, FIG. 5g and FIG. 5i are expressed as mean±sem, n=9 neurons in FIG. 5c, FIG. 5d, FIG. 5g and n=8 neurons in i from 1 male and 1 female mouse at the age of 3-4 weeks. Statistics source data are provided.

FIG. 6. The alignment of KCRs and representative cryptophyte BCCRs. The black lines show the predicted transmembrane helices (TM1-TM7). The Schiff base lysine is highlighted blue, the conserved aspartates corresponding to Asp85 and Asp96 of bacteriorhodopsin, red, and the residues implicated in trimer formation in ChRmine, green. GtCCR2, Guillardia theta CCR2; RaCCR1, Rhodomonas abbreviata CCR1. (SEQ ID NOS: 14-18)

FIGS. 7a-e. Electrophysiological characterization of HcKCR2 in HEK293 cells. FIG. 7a, FIG. 7b, Series of HcKCR2 photocurrents recorded in response to 1-s light pulses under indicated ionic and voltage conditions. FIG. 7c, The IV curves measured under indicated ionic conditions (mean±sem, n=7 cells from 3 independent experiments). FIG. 7d, FIG. 7e, The Vrev values determined for the peak current and current at the end of a 1-s light pulse under indicated ionic conditions. The symbols are data from individual cells, the lines are the mean±sem, n=7 cells from 3 independent experiments. *, P=0.031; n. s. (not significant), P=1 by two-sided paired sample Wilcoxon signed ranks test. Source data are provided.

FIG. 8. Analysis of relative permeabilities of HcKCR1 for metal cations and NMDG+. Left, HcKCR1 peak photocurrents recorded in response to 1-s light pulses with 130 mM KCl in the pipette and 130 mM of the indicated cation in the bath. Right, the corresponding IV curves (mean±sem, n=8 cells from 3 independent experiments). The red lines show linear approximations used to determine the Vrev. Source data are provided.

FIG. 9. Comparison of the Vrev values of HcKCR1 photocurrents measured at the time of the peak and at the end of a 1-s light pulse. The photocurrents were recorded with 130 mM KCl in the pipette and 130 mM of the indicated cation in the bath. The symbols are the data from individual cells, the lines are the mean±sem, n=10 cells for Na+, 7 cells for Li+, Cs+, NMDG+, Ca2+, K+pH 5.4, 6 cells for Mg2+, and 8 cells for K+pH 7.4 and Rb+. P values were determined by two-sided paired sample Wilcoxon signed ranks test. Source data are provided.

FIGS. 10a-f. Light intensity dependence of HcKCR1 photocurrents. FIG. 10a, A series of HcKCR1 photocurrents recorded at 20 mV in response to 200-ms light pulses of the intensity indicated in the legend. The green bar shows the duration of illumination. FIGS. 10b-f, Dependence of peak photocurrent (FIG. 10b), photocurrent at the end of the light pulse (FIG. 10c), desensitization (FIG. 10d), the time constant (T) of photocurrent rise (FIG. 10e), and τ of photocurrent decay (FIG. 10f) on the stimulus intensity. Light gray, data from individual cells, and black, mean±sem (n=5 cells from 3 independent experiments). Source data are provided.

FIGS. 11a-d. Representative photocurrents recorded under two-photon (2P) illumination at different laser powers and wavelengths. FIG. 11a, FIG. 11b, Representative HcKCR1 (FIG. 11a) and HcKCR2 (FIG. 11b) photocurrents recorded at different power levels. FIG. 11c, FIG. 11d, Representative HcKCR1 (FIG. 11c) and HcKCR2 (FIG. 11d) photocurrents recorded with light of different wavelengths. All traces were recorded using KCR-expressing HEK293A cells held at −20 mV. The red bars mark the timing of the 1-s 2P excitation periods. The jagged appearance of the traces is due to raster scanning artifacts (see Methods).

FIGS. 12a-f. Extended analysis of photocurrents and photochemical conversion upon single quantum excitation. FIG. 12a, Photocurrent traces (thin solid lines) recorded from HcKCR1 in response to 6-ns laser flashes at 20-mV voltage increments under indicated ionic conditions and their multiexponential approximations (dashed lines). FIG. 12b, The time constants (T) of the three kinetic components of channel currents at −60 (red) and 0 (black) mV (mean±sem). The data for individual cells are shown as circles; mean±sem, as lines (n=16 cells for fast opening, 13 cells for slow opening at −60 mV, 10 cells for slow opening at 0 mV, and 17 cells for closing). *, P=0.017 **, P=0.06; n.s. (not significant), P=0.877 by the two-sided Mann-Whitney test. Statistics source data are provided. FIG. 12c, The voltage dependence of the three kinetic components of channel currents. FIG. 12d, Peak amplitude channel currents recorded at varied time intervals between laser flashes. The datapoints are mean±sem, n=5 cells. Statistics source data are provided. FIG. 12e, Laser flash-induced absorption changes of HcKCR1 in detergent (black) and Pichia membranes (red). Experimental data are shown as thin solid lines, and their multiexponential approximations, as dashed lines. FIG. 12f, HcKCR1 photocurrent traces in the absence of permeant metal cations at bath pH 5.4. Experimental data are shown as thin solid lines, and their multiexponential approximations, as dashed lines.

FIGS. 13a-b. HcKCR1 expression in mouse cortical neurons. FIG. 13a, Fluorescence images showing HcKCR1-EYFP (green) and tdTomato (magenta) expression in layer 2/3 pyramidal neurons. HcKCR1-EYFP is expressed at high levels and forms some intracellular aggregates, as do many other wild-type ChRs. HcKCR2-EYFP shows the same degree of aggregation. Membrane targeting of both KCRs is confirmed by robust photocurrents (FIG. 5b and FIGS. 14a-g). FIG. 13b, The fluorescence image of HcKCR1-EYFP from FIG. 5a (left panel) was overexposed to visualize the presence of HcKCR1-EYFP in the dendrites and axons (right panel). Note, the axons of layer 2/3 pyramidal neurons ramify in layer 5. Similar results were observed in 14 slices from 2 male and 2 female mice at the age of 3-4 weeks.

FIGS. 14a-g. Photoactivation of HcKCR2 in neurons causes action potentials but can also inhibit neuronal spiking. FIG. 14a, Photocurrent traces of a HcKCR2-expressing neuron in response to a 1-s 470 nm light pulse (18.0 mW mm-2) at holding voltages increased in 5-mV steps. HcKCR2 generated robust photocurrents, but at more negative voltages the onset of illumination caused large inward currents, corresponding to action potentials (boxed segment, see its expansion in FIG. 14b). FIG. 14b, Expanded photocurrent traces of the boxed segment in FIG. 14a. FIG. 14c, FIG. 14d, IV curves of the early photocurrent before the onset of action potentials (FIG. 14c) and the current at the end of illumination (FIG. 14d) of individual neurons indicated by different colors (P<0.0001, R2>0.99 for all linear regressions). FIG. 14e, Reversal potentials calculated from the data in c and d (P=0.1, two-tailed paired t test). The reversal potentials of HcKCR2 are higher than those of HcKCR1, consistent with the results in HEK293 cells (FIGS. 7a-e). FIG. 14f, Membrane voltage traces of a HcKCR2-expressing neuron in response to −0.1 (left), 0 (middle), or 0.5 nA (right) current injections without (top) and with (bottom) 470 nm illumination. When the membrane potential was at rest or depolarized (middle and right), photoactivation of HcKCR2 often caused action potentials at the onset of light because the reversal potential was close to the action potential threshold. Nevertheless, action potentials evoked by current injections were suppressed by long pulses of light (right) due to shunting inhibition. FIG. 14g, The frequencies of action potentials evoked by different current injections with (magenta) and without (black) photoactivation (P>0.99 for all current levels by the Multiple Wilcoxon matched-pairs signed rank test with Bonferroni—Dunn multiple corrections). Data in e and g are expressed as mean±sem, n=3 neurons from 1 male and 1 female mouse at the age of 3-4 weeks.

FIG. 15. HcKCR1 photocurrent recovery in the dark in experiments with 1-s light pulses. Neurons were stimulated by two light pulses (13.1 mW mm-2) applied with a 10-s, 20-s, or 30-s interval, and the ratio of the peak or end currents evoked by the 2nd pulse to that of the 1st pulse was calculated. ITI, intertrial interval. Data are expressed as mean±sem; n=8 neurons from 1 male and 1 female mouse at the age of 3-4 weeks.

### DESCRIPTION OF ILLUSTRATIVE EMBODIMENTS

Until recently channelrhodopsins have been phototaxis receptors that function as light-gated cation channels that when transfected into animal cells, are used for photoactivation of neuron firing. Described herein are new light gated channels, kalium (potassium) channel rhodopsins (KCRs). Different ACRs and uses thereof have been described in, for example, in PCT patent application no: PCT/US2016/023095, incorporated herein by reference. The inventors have now identified a novel set of KCRs that could likewise be used to generate recombinant constructs, such as expression constructs for expressing the KCRs target cells. In some embodiments, the disclosed KCRs can be used in diagnostic and/or therapeutic methods further detailed herein.

Potassium (K+) channels are ubiquitously found in all domains of life. They are easily recognized by their highly conserved K+ channel signature sequence (MacKinnon, 2003; Mironenko et al., 2021) that encodes a K+-selectivity filter that strongly favors conductance of K+ over Na+. Described herein is a unique class of K+ channels, the members of which (i) lack the “K+ channel signature sequence” T(S)VGY(F)G that forms a tetrameric selectivity filter in previously known K+ channels, and (ii) are channelrhodopsins, retinylidene proteins gated by light.

Channelrhodopsins (ChRs) are light-gated ion channels first discovered in Chlamydomonas reinhardtii. They are used to manipulate the membrane potential of excitable animal cells with light (optogenetics). Cation conductive ChRs (CCRs) are primarily proton channels, some of which also conduct metal cations. Photoactivation of CCRs depolarizes the neuronal membrane and stimulates spiking. Anion conductive ChRs (ACRs) conduct halides and nitrate and generate hyperpolarizing or depolarizing photocurrents depending on the electrochemical potential of Cl—. ACRs suppress neuron excitability with hyperpolarizing currents or by shunting inhibition when illuminated at the Cl— reversal potential.

The dissipation of the K+ electrochemical gradient causes membrane hyperpolarization in neurons, which has stimulated efforts to engineer light-gated K+ channels. The K+/Na+ permeability ratio (PK/PNa) of C. reinhardtii ChR2 (CrChR2) is 0.3-0.55, 12 and could be increased no more than twice by mutation (Richards & Dempski, 2021). Alternatively, neuronal K+ channels have been modified by the addition of synthetic photoswitches or photoactive protein domains (Alberio et al., 2018), or controlled indirectly by co-expressing with a photosensitive adenylyl cyclase (Beck et al., 2018, Bernal Siena et al., 2018). Both approaches are promising for some applications but are limited by slow kinetics and possible cAMP-induced side effects. Herein are described two ChRs from Hyphochytrium catenoides, which were named HcKCR1 and HcKCR2 (for Hyphochytrium catenoides kalium channelrhodopsins), are highly specific, robust light-gated K+ channels with rapid kinetics.

### I. DEFINITIONS

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

As used herein, the term “about,” when used in conjunction with a percentage or other numerical amount, means plus or minus 10% of that percentage or other numerical amount. For example, the term “about 80%,” would encompass 80% plus or minus 8%.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls

As used herein, and unless otherwise indicated, the term a disorder that involves electrically active cells, such as but not limited to neuronal dysfunction, a neuron mediated disorder, ocular disorder or cardiac disorder, for which the present methods and compositions may be used include, but are not limited to, neuronal dysfunctions, disorders of the brain, the central nervous system, the peripheral nervous system, neurological conditions, disorders of memory and leaning disorders, cardiac arrhythmias, Parkinson's disease, epilepsy, ocular disorders, spinal cord injury, nerve pain associated with, but not limited to autoimmune diseases (for example, multiple sclerosis, Guillain-Barre syndrome, myasthenia gravis, lupus, and inflammatory bowel disease); cancer and the chemotherapy and radiation used to treat it; compression/trauma (for example, pinched nerves in the neck, crush injuries, and carpal tunnel syndrome); diabetic neuropathy; medication side effects; and toxic substances; motor neuron diseases (for example amyotrophic lateral sclerosis, progressive bulbar palsy, progressive muscular atrophy and primary lateral sclerosis); nutritional deficiencies (for example vitamins B6 and B12); Infectious disease; itch sensations associated with, but not limited to eczema, atopic dermatitis, dry skin and allergic itches; diseases and disorders that alter vagal nerve activity, among others. In particular embodiments are potassium channelopathies such as those associated with epilepsy, Parkinson's disease, and long-QT syndrome and other cardiac arrhythmias.

As used herein, and unless otherwise indicated, the term ocular disorders for which the present methods and compositions may be used to improve one or more parameters of vision include, but are not limited to, developmental abnormalities that affect both anterior and posterior segments of the eye. Anterior segment disorders include, but are not limited to, glaucoma, cataracts, corneal dystrophy, keratoconus. Posterior segment disorders include, but are not limited to, blinding disorders caused by photoreceptor malfunction and/or death caused by retinal dystrophies and degenerations. Retinal disorders include congenital stationary night blindness, age-related macular degeneration, congenital cone dystrophies, and a large group of retinitis-pigmentosa (RP)—related disorders.

As used herein, and unless otherwise indicated, the terms “treat,” “treating,” “treatment” and “therapy” contemplate an action that occurs while a patient is suffering from a disorder that involves electrically active cells, such as but not limited to neuronal dysfunction, a neuron mediated disorder, ocular disorder or cardiac disorder, and which reduces the severity of one or more symptoms or effect of such a disorder. Where the context allows, the terms “treat,” “treating,” and “treatment” also refers to actions taken toward ensuring that individuals at increased risk of a disorder that involves electrically active cells, such as but not limited to neuronal dysfunction, a neuron mediated disorder, ocular disorder or cardiac disorder and which reduces the severity are able to receive appropriate surgical and/or other medical intervention prior to onset of a disorder that involves electrically active cells, such as but not limited to neuronal dysfunction, a neuron mediated disorder, ocular disorder or cardiac disorder and which reduces the severity. As used herein, and unless otherwise indicated, the terms “prevent,” “preventing,” and “prevention” contemplate an action that occurs before a patient begins to suffer from a disorder that involves electrically active cells, such as but not limited to neuronal dysfunction, a neuron mediated disorder, ocular disorder or cardiac disorder, that delays the onset of, and/or inhibits or reduces the severity of a disorder that involves electrically active cells, such as but not limited to neuronal dysfunction, a neuron mediated disorder, ocular disorder or cardiac disorder. In particular embodiments are potassium channelopathies such as those associated with epilepsy, Parkinson's disease, and long-QT syndrome and other cardiac arrhythmias.

As used herein, and unless otherwise indicated, the terms “manage,” “managing,” and “management” encompass preventing, delaying, or reducing the severity of a recurrence of a disorder that involves electrically active cells, such as but not limited to neuronal dysfunction, a neuron mediated disorder, ocular disorder or cardiac disorder in a patient who has already suffered from such a disease, disorder or condition. The terms encompass modulating the threshold, development, and/or duration of the disorder that involves electrically active cells or changing how a patient responds to the disorder that involves electrically active cells or the maintenance and/or establishment of a desirable membrane potential across the membrane of a cell.

As used herein, and unless otherwise specified, a “therapeutically effective amount” of a compound is an amount sufficient to provide any therapeutic benefit in the treatment or management of a disorder that involves electrically active cells, such as but not limited to neuronal dysfunction, a neuron mediated disorder, ocular disorder or cardiac disorder, or to delay or minimize one or more symptoms associated with a disorder that involves electrically active cells, such as but not limited to neuronal dysfunction, a neuron mediated disorder, ocular disorder or cardiac disorder. A therapeutically effective amount of a compound means an amount of the compound, alone or in combination with one or more other therapies and/or therapeutic agents that provide any therapeutic benefit in the treatment or management of a disorder that involves electrically active cells, such as but not limited to neuronal dysfunction, a neuron mediated disorder, ocular disorder or cardiac disorder. In particular embodiments are therapies directed at potassium channelopathies such as those associated with epilepsy, Parkinson's disease, and long-QT syndrome and other cardiac arrhythmias.

As used herein, and unless otherwise specified, a “prophylactically effective amount” of a compound is an amount sufficient to prevent or delay the onset of a disorder that involves electrically active cells, such as but not limited to neuronal dysfunction, a neuron mediated disorder, ocular disorder or cardiac disorder or one or more symptoms associated with a disorder that involves electrically active cells or prevent or delay its recurrence. A prophylactically effective amount of a compound means an amount of the compound, alone or in combination with one or more other treatment and/or prophylactic agent that provides a prophylactic benefit in the prevention of a disorder that involves electrically active cells, such as but not limited to neuronal dysfunction, a neuron mediated disorder, ocular disorder or cardiac disorder. The term “prophylactically effective amount” can encompass an amount that prevents a disorder that involves electrically active cells, such as but not limited to neuronal dysfunction, a neuron mediated disorder, ocular disorder or cardiac disorder, improves overall prophylaxis, or enhances the prophylactic efficacy of another prophylactic agent. The “prophylactically effective amount” can be prescribed prior to, for example, the development of a disorder that involves electrically active cells, such as but not limited to neuronal dysfunction, a neuron mediated disorder, ocular disorder or cardiac disorder. In particular embodiments are potassium channelopathies such as those associated with epilepsy, Parkinson's disease, and long-QT syndrome and other cardiac arrhythmias.

As used herein, “patient” or “subject” includes mammalian organisms which are capable of suffering from a disorder that involves electrically active cells, such as but not limited to neuronal dysfunction, a neuron mediated disorder, ocular disorder or cardiac disorder, as described herein, such as human and non-human mammals, for example, but not limited to, rodents, mice, rats, non-human primates, companion animals such as dogs and cats as well as livestock, e.g., sheep, cow, horse, etc.

As used herein, the term “conservative substitution” generally refers to amino acid replacements that preserve the structure and functional properties of a protein or polypeptide. Such functionally equivalent (conservative substitution) peptide amino acid sequences include, but are not limited to, additions or substitutions of amino acid residues within the amino acid sequences encoded by a nucleotide sequence that result in a silent change, thus producing a functionally equivalent gene product. Conservative amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

As used herein, a “redshift” is a shift to longer wavelength. In contrast a “blueshift” would be a shift to shorter wavelength. These terms apply to both light-emitting and light-absorbing objects.

As used herein the phrase “rhodopsin domain” refers to the “rhodopsin fold”, a 7-transmembrane-helix (7TM) structure characteristic of rhodopsins. As used herein, the channelopsin is the apoprotein, while channelrhodopsin is the protein and retinal. As used herein the term “channelrhodopsin” describes retinylidene proteins (rhodopsins) that function as light-gated ion channels.

The percent identity or homology is determined with regard to the length of the relevant amino acid sequence. Therefore, if a polypeptide of the present invention is comprised within a larger polypeptide, the percent homology is determined with regard only to the portion of the polypeptide that corresponds to the polypeptide of the present invention and not the percent homology of the entirety of the larger polypeptide. “Percent identity” or “% identity,” with reference to nucleic acid sequences, refers to the percentage of identical nucleotides between at least two polynucleotide sequences aligned using the Basic Local Alignment Search Tool (BLAST) engine. See Tatusova et al., 1999.

“Percent identity” or “% identity,” with reference to polypeptide sequences, refers to the percentage of identical amino acids between at least two polypeptide sequences aligned using the Basic Local Alignment Search Tool (BLAST) engine. See Tatusova et al. (1999).

A. Potassium-Conducting Channelrhodopsins

Sequences encoding 7TM domain channelrhodopsins provided herein. In particular, previously unknown potassium-conducting channelrhodopsins having the amino acid sequence of SEQ ID NO: 1 or SEQ ID NO: 2, or encoded by the polynucleotide sequence of SEQ ID NO: 3, SEQ ID NO: 4, SEQ ID NO: 5, or SEQ ID NO: k are provided.

In some embodiments, are conserved variants of functional humanized rhodopsin domain or a peptide fragment thereof. A “conservative” amino acid substitution refers to the substitution of an amino acid in a polypeptide with another amino acid having similar properties, such as size or charge. In certain embodiments, a polypeptide comprising a conservative amino acid substitution maintains at least one activity of the unsubstituted polypeptide. A conservative amino acid substitution may encompass non-naturally occurring amino acid residues which are typically incorporated by chemical peptide synthesis rather than by synthesis in biological systems. These include, but are not limited to, peptidomimetics and other reversed or inverted forms of amino acid moieties.

In some embodiments, are any of the disclosed methods, wherein the rhodopsin domain of a potassium-conducting channelrhodopsin having the amino acid sequence of all or part of SEQ ID NO: 1 or SEQ ID NO: 2 or a biologically active fragment thereof that retains the biological activity of the encoded rhodopsin domain of a potassium-conducting channelrhodopsin or a biologically active conservative amino acid substitution variant of SEQ ID NO: 1 or SEQ ID NO: 2 or of said fragment.

B. Potassium-Conducting Channelrhodopsin Polypeptides

In some embodiments, are isolated polypeptides that encode a rhodopsin domain of a potassium-conducting channelrhodopsin. In some embodiments, an isolated polypeptide comprising the amino acid sequence of SEQ ID NO: 1 or SEQ ID NO: 2. In some embodiments, the isolated polypeptide has at least 85% homology to the amino acid sequence of SEQ ID NO: 1 or SEQ ID NO: 2. In some embodiments, the isolated polypeptide has between 85%-95%-100% homology to the amino acid sequence of SEQ ID NO: 1 or SEQ ID NO: 2.

In some embodiments, is a protein composition comprises a polypeptide having the amino acid sequence of SEQ ID NO: 1 or SEQ ID NO: 2. The peptide amino acid sequences that can be used in various embodiments including the potassium-conducting rhodopsin domain amino acid sequences described herein (SEQ ID NO: 1 or SEQ ID NO: 2), as well as analogues and derivatives thereof and functional fragments such as but not limited to the rhodopsin/7TM domain. In fact, in some embodiments the any desired peptide amino acid sequences encoded by particular nucleotide sequences can be used, as is the use of any polynucleotide sequences encoding all, or any portion, of desired peptide amino acid sequences. The degenerate nature of the genetic code is well-known, and, accordingly, each potassium-conducting rhodopsin domain peptide amino acid-encoding nucleotide sequence is generically representative of the well-known nucleic acid “triplet” codon, or in many cases codons, that can encode the amino acid. As such, as contemplated herein, the kalium (potassium)-conducting channelrhodopsin peptide amino acid sequences described herein, when taken together with the genetic code (see, e.g., “Molecular Cell Biology”, Table 4-1 at page 109 (Darnell et al., 1986)), are generically representative of all the various permutations and combinations of nucleic acid sequences that can encode such amino acid sequences.

Such functionally equivalent peptide amino acid sequences (conservative substitutions) include, but are not limited to, additions or substitutions of amino acid residues within the amino acid sequences encoded by a nucleotide sequence, but that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Naturally occurring residues may be divided into classes based on common side chain properties: hydrophobic (Met, Ala, Val, Leu, Ile); neutral hydrophilic (Cys, Ser, Thr, Asn, Gln); acidic (Asp, Glu); basic (His, Lys, Arg); residues that influence chain orientation (Gly, Pro); and aromatic (Trp, Tyr, Phe). For example, non-conservative substitutions may involve the exchange of a member of one of these classes for a member from another class.

In making substitutions, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, in certain instances, is understood in the art (Kyte et al., 1982). It is known that in certain instances, certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

Substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included.

A skilled artisan will be able to determine suitable variants of a polypeptide as set forth herein using well-known techniques. In certain embodiments, one skilled in the art may identify suitable areas of the molecule that may be changed without destroying activity by targeting regions not believed to be important for activity. In certain embodiments, one can identify residues and portions of the molecules that are conserved among similar polypeptides.

In certain embodiments, even areas that may be important for biological activity or for structure may be subject to conservative amino acid substitutions without destroying the biological activity or without adversely affecting the polypeptide structure.

Additionally, in certain embodiments, one skilled in the art can review structure-function studies identifying residues in similar polypeptides that are important for activity or structure. In view of such a comparison, in certain embodiments, one can predict the importance of amino acid residues in a protein that correspond to amino acid residues which are important for activity or structure in similar proteins. In certain embodiments, one skilled in the art may opt for chemically similar amino acid substitutions for such predicted important amino acid residues.

In certain embodiments, one skilled in the art can also analyze the three-dimensional structure and amino acid sequence in relation to that structure in similar polypeptides. In certain embodiments, in view of such information, one skilled in the art may predict the alignment of amino acid residues of a polypeptide with respect to its three-dimensional structure. In certain embodiments, one skilled in the art may choose not to make radical changes to amino acid residues predicted to be on the surface of the protein, since such residues may be involved in important interactions with other molecules.

Moreover, in certain embodiments, one skilled in the art may generate test variants containing a single amino acid substitution at each desired amino acid residue. In certain embodiments, the variants can then be screened using activity assays known to those skilled in the art. In certain embodiments, such variants could be used to gather information about suitable variants. For example, in certain embodiments, if one discovered that a change to a particular amino acid residue resulted in destroyed, undesirably reduced, or unsuitable activity, variants with such a change may be avoided. In other words, in certain embodiments, based on information gathered from such routine experiments, one skilled in the art can readily determine the amino acids where further substitutions should be avoided either alone or in combination with other mutations.

A number of scientific publications have been devoted to the prediction of secondary structure. See, e.g., (Moult, 1996; Chou et al., 1974a; Chou et al., 1974b; Chou et al., 1978; Chou et al., 1979a; Chou et al., 1979b). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The growth of the protein structural database (PDB) has provided enhanced predictability of secondary structure, including the potential number of folds within a polypeptide's structure. See, e.g., Holm et al., 1999. It has been suggested (Brenner et al., 1997) that there are a limited number of folds in a given polypeptide or protein and that once a critical number of structures have been resolved, structural prediction will become dramatically more accurate.

Additional methods of predicting secondary structure include “threading” (see, e.g., Jones, 1997; Sippl et al., 1996), “profile analysis” (see, e.g., Bowie et al., 1991); Gribskov et al., 1990); Gribskov et al., 1987), and “evolutionary linkage” (see, e.g., Holm et al., 1999), and Brenner et al., 1997).

In certain embodiments, a variant of the reference channelrhodopsin or rhodopsin domain, such as those encoded by SEQ ID NO: 2 or SEQ ID NO: 4 includes a glycosylation variant wherein the number and/or type of glycosylation sites have been altered relative to the amino acid sequence of the reference kalium (potassium)-conducting channelrhodopsin or rhodopsin domain. In certain embodiments, a variant of a polypeptide comprises a greater or a lesser number of N-linked glycosylation sites relative to a native polypeptide. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. In certain embodiments, a rearrangement of N-linked carbohydrate chains is provided, wherein one or more N-linked glycosylation sites (typically those that are naturally occurring) are eliminated and one or more new N-linked sites are created. Exemplary variants include cysteine variants wherein one or more cysteine residues are deleted from or substituted for another amino acid (e.g., serine) relative to the amino acid sequence of the reference channelrhodopsin or rhodopsin domain (e.g., such as those provided as SEQ ID NO: 1 or SEQ ID NO: 2). In certain embodiments, cysteine variants may be useful when polypeptides and proteins must be refolded into a biologically active conformation such as after the isolation of insoluble inclusion bodies. In certain embodiments, cysteine variants have fewer cysteine residues than the native polypeptide. In certain embodiments, cysteine variants have an even number of cysteine residues to minimize interactions resulting from unpaired cysteines.

According to certain embodiments, amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in a naturally-occurring sequence (in certain embodiments, in the portion of the polypeptide outside the domain(s) forming intermolecular contacts). In certain embodiments, a conservative amino acid substitution typically may not substantially change the structural characteristics of the reference sequence (e.g., in certain embodiments, a replacement amino acid should not tend to break a helix that occurs in the reference sequence or disrupt other types of secondary structure that characterizes the reference sequence).

Examples of certain art-recognized polypeptide secondary and tertiary structures are described, for example, in Proteins, Structures and Molecular Principles (Creighton & Freeman 1984); Introduction to Protein Structure (Branden & Tooze, 1991); and Thornton et al., 1991.

In other embodiments, are methods and compositions that provide an KCR with improved properties and characteristics that enhance the application of the compositions in, among other things, optogenetic techniques and therapies directed at potassium channelopathies such as those associated with epilepsy, Parkinson's disease, and long-QT syndrome and other cardiac arrhythmias.

Improved properties include, those described herein, such as adaptation to human codon usage and synthesis. These embodiments provide greater sensitivity and efficient membrane hyperpolarization and neuronal silencing through light-gated cation potassium conduction.

C. Fusion Proteins

The use of fusion proteins in which a polypeptide or peptide, or a truncated or mutant version of peptide is fused to an unrelated or homologous protein, polypeptide, or peptide, and can be designed on the basis of the desired peptide encoding nucleic acid and/or amino acid sequences described herein. Such fusion proteins include, but are not limited to: IgFc fusions, which stabilize proteins or peptides and prolong half-life in vivo; fusions to any amino acid sequence that allows the fusion protein to be anchored to the cell membrane; or fusions to an enzyme, fluorescent protein, or luminescent protein that provides a marker function. Fusion proteins to homologous proteins include, but are not limited to, those that are produced from genes that are engineered to encode a portion of the kalium-conducting channelrhodopsin fused to a portion of a homologous (orthologous or paralogous) protein of the same of related function. For example, chimeras between different channelrhodopsins may be made to combine beneficial properties uniquely present in each. In some aspects, a chimeric channelrhodopsin of the embodiments comprises about 50%, 55%, 60%, 65%, 70%, 75%, 80%, 85%, 90% or 95% of its sequence from a first channelrhodopsin and the remaining sequence from a second channelrhodopsin. In some aspects, a chimeric channelrhodopsin comprises the rhodopsin domain of a first channelrhodopsin and the remaining sequence from a second channelrhodopsin. In yet further aspects, a chimeric channelrhodopsin can comprise 1, 2, 3, 4, 5 or 6 of its transmembrane domains from a first channelrhodopsin and the remaining transmembrane domains from a second channelrhodopsin.

In certain embodiments, a fusion protein may be readily purified by utilizing an antibody that selectively binds to the fusion protein being expressed. In alternate embodiments, a fusion protein may be purified by subcloning peptide encoding nucleic acid sequence into a recombination plasmid, or a portion thereof, is translationally fused to an amino-terminal (N-terminal) or carboxy-terminal (C-terminal) tag consisting of six histidine residues (a “His-tag”; see, e.g., Janknecht et al., 1991). Extracts from cells expressing such a construct are loaded onto Ni²⁺ nitriloacetic acid-agarose columns, and histidine-tagged proteins are selectively eluted with imidazole-containing buffers.

D. Nucleic Acids Encoding Channelrhodopsins

In some embodiments, a recombinant nucleic acid operatively linked to a heterologous promoter sequence, said recombinant nucleic acid comprising: a sequence that encodes a peptide with at least 85% homology to an amino acid sequence selected from SEQ ID NO: 1 or SEQ ID NO: 2 or a sequence that encodes a peptide comprising 225 contiguous amino acids selected from SEQ ID NO: 1 or SEQ ID NO: 2; or a sequence that hybridizes to the nucleotide sequence of SEQ ID NO: 3, SEQ ID NO: 4, SEQ ID NO: 5, SEQ ID NO: 6, or the complement thereof.

In some embodiments, are isolated nucleic acid molecules comprising a nucleotide sequence that encode a kalium-conducting channelrhodopsin. In some embodiments, the rhodopsin domain encodes the peptides whose sequence is described in SEQ ID NO: 1 or SEQ ID NO: 2. In some embodiments, are isolated nucleic acid molecules comprising a nucleotide sequence that encodes the amino acid sequence shown in SEQ ID NO: 3, SEQ ID NO: 4, SEQ ID NO: 5 or SEQ ID NO: 6. In some embodiments, are expression vectors comprising a nucleic acid sequence that encodes the amino acid sequences of SEQ ID NO: 1 or SEQ ID NO: 2. In some embodiments, are host cells comprising an expression vector comprising a nucleic acid sequence that encodes the amino acid sequences of SEQ ID NO: 1 or SEQ ID NO: 2 or a nucleic acid sequence, fragment of portion thereof of the nucleic acid sequences of SEQ ID NO: 3, SEQ ID NO: 4, SEQ ID NO: 5 or SEQ ID NO: 6.

In some embodiments, are isolated nucleic acid molecules comprising a nucleotide sequence that was derived from cDNA and encode the rhodopsin domain of an KCR. In some embodiments, the rhodopsin domain encodes the peptides whose sequence is described in SEQ ID NO: 1 or SEQ ID NO: 2. In some embodiments, are isolated nucleic acid molecules that were derived from cDNA that comprise a nucleotide sequence that encodes the amino acid sequence shown in SEQ ID NO: 1 or SEQ ID NO: 2. In some embodiments, are expression vectors comprising a nucleic acid sequence that encodes the amino acid sequences of SEQ ID NO: 1 or SEQ ID NO: 2. In some embodiments, are host cells comprising a recombinant expression vector comprising a nucleic acid sequence that was derived from cDNA and encode the amino acid sequences of SEQ ID NO: 1 or SEQ ID NO: 2.

In some embodiments, isolated nucleic acid molecules are provided comprising a nucleotide sequence that encodes the rhodopsin domain of a kalium-conducting channelrhodopsin. In some embodiments, the rhodopsin encodes a peptide whose sequence is shown in SEQ ID NO: 1 or SEQ ID NO: 2. In some embodiments, are isolated nucleic acid molecules comprising a nucleotide sequence that encodes the amino acid sequence shown in SEQ ID NO: 1 or SEQ ID NO: 2. In some embodiments, are expression vectors comprising a nucleic acid sequence that encodes the amino acid sequences of SEQ ID NO: 1 or SEQ ID NO: 2. In some embodiments, are host cells comprising a expression vector comprising a nucleic acid sequence that encodes the amino acid sequences of SEQ ID NO: 1 or SEQ ID NO: 2. In some embodiments, are peptides comprising a sequence that encodes the rhodopsin domain of an kalium-conducting channelrhodopsin. In some embodiments, are isolated peptides comprising an amino acid sequence of SEQ ID NO: 1 or SEQ ID NO: 2. In some embodiments, the isolated peptides comprise an amino acid sequence of SEQ ID NO: 1 or SEQ ID NO: 2, or fragments thereof.

In some embodiments, are isolated nucleic acid molecules wherein said nucleic acid molecule has a sequence of SEQ ID NO: 3, SEQ ID NO: 4, SEQ ID NO: 5 or SEQ ID NO: 6. In other embodiments, are expression vectors comprising a nucleic acid sequence selected from that shown in SEQ ID NO: 3, SEQ ID NO: 4, SEQ ID NO: 5 or SEQ ID NO: 6 and those that encode the amino acid sequences shown in SEQ ID NO: 1 or SEQ ID NO: 24. In some embodiments, are host cells comprising an expression vector comprising a nucleic acid sequence shown in SEQ ID NO: 3, SEQ ID NO: 4, SEQ ID NO: 5 or SEQ ID NO: 6 and those that encode the amino acid sequences of SEQ ID NO: 1 or SEQ ID NO: 2. In some embodiments, an isolated nucleic acid comprises a nucleotide sequence that encodes the rhodopsin domain of a potassium-conducting channelrhodopsin. In some embodiments, the nucleotide sequence encodes at least 16, 20, 33, 35, or 75 contiguous amino acids of SEQ ID NO: 1 or SEQ ID NO: 2. In some embodiments, the nucleotide sequence encodes a peptide comprising any contiguous portion of SEQ ID NO: 1 or SEQ ID NO: 2.

In some embodiments, an isolated nucleic acid comprising a nucleotide sequence that encodes a functional domain of a kalium-conducting channelrhodopsin. In some embodiments are isolated nucleic acid that encodes at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 150, 175, 200, 205, 210, 215, 220, 225, 228, 229, 230, 235, 240 250, 255, 260, 265, 270, 275, 280, 285, 290, 295, 296 or more contiguous amino acids of SEQ ID NO: 1 or SEQ ID NO: 2. Further, in some embodiments, any range derivable between any of the above-described integers.

In other embodiments, there is provided an isolated polypeptide or an isolated nucleic acid encoding a polypeptide having in some embodiments between about 70% and about 75%; in further embodiments between about 75% and about 80%; in further still embodiments between about 80% and 90%; or even more further between about 90% and about 99% of amino acids (for example 95%) that are identical to (or homologous to) the amino acids of SEQ ID NO: for SEQ ID NO: 2, or fragments thereof.

In other embodiments, the present invention provides for an isolated nucleic acid encoding a polypeptide having between about 70% and about 75%; or more preferably between about 75% and about 80%; or more preferably between about 80% and 90%; or even more preferably between about 90% and about 99% of amino acids that are identical to the amino acids of SEQ ID NO: 1 or SEQ ID NO: 2, or fragments thereof.

In some embodiments, the nucleic acid segments, regardless of the length of the coding sequence itself, may be combined with other DNA sequences, such as promoters, enhancers, polyadenylation signals, additional restriction enzyme sites, multiple cloning sites, other coding segments, and the like. In some embodiments, for example, are recombinant nucleic acids comprising a nucleotide sequence that encodes amino acids of SEQ ID NO: 1 or SEQ ID NO: 2 or fragments thereof, operably linked to a heterologous promoter.

In certain embodiments the invention provides an isolated nucleic acid obtained by amplification from a template nucleic acid using a primer selected from appropriate primer that can be used with SEQ ID NO: 3, SEQ ID NO: 4, SEQ ID NO: 5 or SEQ ID NO: 6.

In some embodiments, are any of the disclosed methods wherein the expression vectors include, but are not limited to, AAV viral vector. In some embodiments, are any of the disclosed methods wherein the promoter is a constitutive promoter. In some embodiments, are any of the disclosed methods wherein the constitutive promoter includes, but is not limited to, a CMV promoter or a hybrid CMV enhancer/chicken β-actin (CAG) promoter. In some embodiments, are any of the disclosed methods wherein the promoter includes, but is not limited to, an inducible and/or a cell type-specific promoter.

In some embodiments, there is provided a cDNA-derived nucleic acid comprising a nucleic acid sequence that encodes an amino acid sequence selected from SEQ ID NO: 1 or SEQ ID NO: 2 wherein the cDNA-derived nucleic acid comprises the nucleic acid sequence of SEQ ID NO: 3, SEQ ID NO: 4, SEQ ID NO: 5 or SEQ ID NO: 6. In other embodiments is an expression vector comprising the cDNA-derived nucleic acid comprising a nucleic acid sequence that encodes an amino acid sequence selected from SEQ ID NO: 1 or SEQ ID NO: 2.

Rhodopsin domain nucleic acid sequences for use in the disclosed methods and compositions include, but are not limited to, the active portion of the rhodopsin domains presently disclosed and encoded by the nucleic acid sequences of SEQ ID NO: 3, SEQ ID NO: 4, SEQ ID NO: 5 or SEQ ID NO: 6.

In some embodiments, the use of an active portion of a presently disclosed kalium-conducting channelrhodopsin, such as but not limited to the rhodopsin domain, includes all or portions of the sequences described herein (and expression vectors comprising the same), and additionally contemplates the use of any nucleotide sequence encoding a contiguous an active portion of the presently disclosed kalium-conducting channelrhodopsins, such as but not limited to the rhodopsin domain, open reading frame (ORF) that hybridizes to a complement of a kalium-conducting channelrhodopsin or channelopsin sequence described herein under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel et al., 1989), and encodes a functionally equivalent kalium-conducting channelrhodopsin (or active portion thereof, such as but not limited to the rhodopsin domain) gene product or the active portion thereof. Additionally, contemplated is the use of any nucleotide sequence that hybridizes to the complement of a DNA sequence that encodes a kalium-conducting channelrhodopsin amino acid sequence under moderately stringent conditions, e.g., washing in 0.2×SSC/0.1% SDS at 42° C. (Ausubel et al., 1989), yet still encodes a functionally equivalent kalium-conducting channelrhodopsin product. Functional equivalents of kalium-conducting channelrhodopsin include, but are not limited to, naturally occurring versions of kalium-conducting channelrhodopsin present in other or the same species (orthologs, paralogs and more generally homologs), and mutant versions of kalium-conducting channelrhodopsin, whether naturally occurring or engineered (by site directed mutagenesis, gene shuffling, or directed evolution, as described in, for example, U.S. Pat. No. 5,837,458) or active portion thereof, such as but not limited to the rhodopsin domain. The disclosure also includes the use of degenerate nucleic acid variants (due to the redundancy of the genetic code) of the identified channelrhodopsin polynucleotide sequences.

Additionally contemplated is the use of polynucleotides encoding kalium-conducting channelrhodopsin ORFs, or their functional equivalents, encoded by polynucleotide sequences that are about 99%, 95%, 90%, or about 85% similar to the corresponding regions of the kalium-conducting channelrhodopsin sequences described herein (as measured by BLAST sequence comparison analysis using, for example, the University of Wisconsin GCG sequence analysis package (SEQUENCHER 3.0, Gene Codes Corporation, Ann Arbor, MI) using default parameters).

The nucleic acid segments of the embodiments, regardless of the length of the coding sequence itself, may be combined with other sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, nucleic acid fragments may be prepared which include a short stretch complementary to nucleic acids that encode the polypeptides of SEQ ID NO: 1 or SEQ ID NO: 2, such as about 10 to 15 or 20, 30, or 40 or so nucleotides, and which are up to 2000 or so base pairs in length. DNA segments with total lengths of about 8000, 7000, 6000, 5000, 4000, 3000, 2000, 1000, 500, 200, 100 and about 50 base pairs in length are also contemplated to be useful.

In some embodiments, isolated nucleic acids that encode the amino acids of a channelrhodopsin or fragment thereof and recombinant vectors incorporating nucleic acid sequences which encode a channelrhodopsin protein or peptide and that includes within its amino acid sequence an amino acid sequence in accordance with SEQ ID NO: 1 or SEQ ID NO: 2. In some embodiments, a purified nucleic acid segment that encodes a protein that encodes a channelrhodopsin or fragment thereof, the recombinant vector may be further defined as an expression vector comprising a promoter operatively linked to said channelrhodopsin-encoding nucleic acid segment.

In additional embodiments, is a host cell, made recombinant with a recombinant vector comprising channelrhodopsin-encoding nucleic acid segments. The recombinant host cell may be a prokaryotic cell or a eukaryotic cell. As used herein, the term “engineered” or “recombinant” cell is intended to refer to a cell into which a recombinant gene, such as a gene encoding a channelrhodopsin, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a copy of a genomic gene or a cDNA gene, or will include genes positioned adjacent to a promoter not naturally associated with the particular introduced gene. In some embodiments, nucleic acid molecules having sequence regions consisting of contiguous nucleotide stretches of about 14, 15-20, 30, 40, 50, or even of about 100 to about 200 nucleotides or so, identical or complementary to the channelrhodopsin-encoding nucleic acid sequences.

In still further aspects, there is provided a host cell comprising a nucleic acid molecule of the embodiments (e.g., that encodes a KCR). Thus, in some embodiments a host cell is an: isolated human cell; a non-human mammalian cell; a bacterial cell; a yeast cell; an insect cell; or a plant cell. In alternative embodiments, the nucleic acid sequences described can be targeted to the genome of a cell using a CRISPR-associated protein-9 nuclease (Cas9) based system for genome-editing and genome targeting. In some embodiments, delivery to some cells may require delivery systems, such as, but not limited to those based on lentivirus (LVs), adenovirus (AdV) and adenoassociated (AAV).

An exemplary DNA binding protein is an RNA guided DNA binding protein of a Type II CRISPR System. An exemplary DNA binding protein is a Cas9 protein. According to one aspect, an engineered Cas9-gRNA system is provided which enables RNA-guided genome cutting in a site-specific manner, if desired, and modification of the genome by insertion of exogenous channelrhodopsin-encoding nucleic acids provided herein. The guide RNAs are complementary to target sites or target loci on the DNA. The guide RNAs can be crRNA-tracrRNA chimeras. The Cas9 binds at or near target genomic DNA. The one or more guide RNAs bind at or near target genomic DNA. The Cas9 cuts the target genomic DNA and exogenous donor DNA is inserted into the DNA at the cut site.

Accordingly, methods are directed to the use of a guide RNA with a Cas9 protein and an exogenous channelrhodopsin-encoding nucleic acid to multiplex insertions of exogenous channelrhodopsin-encoding nucleic acids into DNA within a cell expressing Cas9 by cycling the insertion of nucleic acid encoding the RNA and exogenous donor nucleic acid, expressing the RNA, colocalizing the RNA, Cas9 and DNA in a manner to cut the DNA, and insertion of the exogenous donor nucleic acid. The method steps can be cycled in any desired number to result in any desired number of DNA modifications.

E. Recombinant Expression

While the desired peptide amino acid sequences described can be chemically synthesized (see, e.g., Creighton, 1984), large polypeptides sequences may advantageously be produced by recombinant DNA technology using techniques well-known in the art for expressing nucleic acids containing a nucleic acid sequence that encodes the desired peptide. Such methods can be used to construct expression vectors containing peptide encoding nucleotide sequences and appropriate transcriptional and translational control signals. These methods include, for example, in vitro recombinant DNA techniques, synthetic techniques, and in vivo genetic recombination (see, e.g., Ausubel et al., 1989). Alternatively, RNA and/or DNA encoding desired peptide encoding nucleotide sequences may be chemically synthesized using, for example, synthesizers (see, e.g., Gait, 1984).

A variety of host-expression vector systems may be utilized to express peptide encoding nucleotide sequences. When the desired peptide or polypeptide is soluble or a soluble derivative, the peptide or polypeptide can be recovered from the host cell culture, i.e., from the host cell in cases where the peptide or polypeptide is not secreted, and from the culture media in cases where the peptide or polypeptide is secreted by the host cell. However, suitable expression systems also encompass engineered host cells that express the desired polypeptide or functional equivalents anchored in the cell membrane. Purification or enrichment of the desired peptide from such expression systems can be accomplished using appropriate detergents and lipid micelles, and methods well-known to those skilled in the art. Furthermore, such engineered host cells themselves may be used in situations where it is desired not only to retain the structural and functional characteristics of the peptide, but to assess biological activity, e.g., in certain drug screening assays.

In certain applications, transient expression systems are desired. However, for long-term, high-yield production of recombinant proteins or peptides, stable expression is generally preferred. For example, cell lines that stably express the desired protein, polypeptide, peptide, or fusion protein may be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells are allowed to grow for about 1-2 days in an enriched media, and then switched to a selective media. The selectable marker in the recombinant plasmid confers resistance to the selection, and allows cells to stably integrate the plasmid into their chromosomes and grow to form foci, which in turn can be cloned and expanded into cell lines. This method may advantageously be used to engineer cell lines that express the desired gene products or portions thereof. Such engineered cell lines may be particularly useful in screening and evaluation of compounds that affect the endogenous activity of a desired protein, polypeptide or peptide.

A number of selection systems may be used, including, but not limited to, the herpes simplex virus thymidine kinase (Wigler et al., 1977), hypoxanthine-guanine phosphoribosyltransferase (Szybalska & Szybalski, 1962), and adenine phosphoribosyltransferase (Lowy et al., 1980) genes, which can be employed in tk⁻, hgprt⁻ or aprt⁻ cells, respectively. Anti-metabolite resistance can also be used as the basis of selection for the following genes: dihydrofolate reductase (dhfr), which confers resistance to methotrexate (Wigler et al., 1980; O'Hare et al., 1981); guanine phosphoribosyl transferase (gpt), which confers resistance to mycophenolic acid (Mulligan & Berg, 1981); neomycin phosphotransferase (neo), which confers resistance to the aminoglycoside G-418 (Colbere-Garapin et al., 1981); and hygromycin B phosphotransferase (hpt), which confers resistance to hygromycin (Santerre et al., 1984).

Host cells/expression systems that may be used for purpose of providing compositions to be used in the disclosed methods include, but are not limited to, microorganisms such as bacteria (e.g., E. coli, B. subtilis) transformed with a recombinant bacteriophage DNA, plasmid DNA, or cosmid DNA expression vector containing a desired peptide encoding nucleotide sequence; yeast (e.g., Saccharomyces cerevisiae, Pichia pastoris) transformed with a recombinant yeast expression vector containing a desired peptide encoding nucleotide sequence; insect cell systems infected with a recombinant virus expression vector (e.g., baculovirus) containing a desired peptide encoding nucleotide sequence; plant cell systems infected with a recombinant virus expression vector (e.g., cauliflower mosaic virus, CaMV; tobacco mosaic virus, TMV), or transformed with a recombinant plasmid expression vector (e.g., Ti plasmid), containing a desired peptide encoding nucleotide sequence; or mammalian cell systems (e.g., COS, CHO, BHK, 293, 3T3) harboring a recombinant expression construct containing a desired peptide encoding nucleotide sequence and a promoter derived from the genome of mammalian cells (e.g., metallothionein promoter) or from mammalian viruses (e.g., the adenovirus late promoter, the vaccinia virus 7.5K promoter).

In bacterial systems, a number of different expression vectors may be advantageously selected depending upon the use intended for the desired gene product being expressed. For example, when a large quantity of such a protein is to be produced, such as for the generation of pharmaceutical compositions comprising a desired peptide, or for raising antibodies to the protein, vectors that direct the expression of high levels of fusion protein products that are readily purified may be desirable. Such vectors include, but are not limited to: the E. coli expression vector pUR278 (Ruther & Müller-Hill, 1983), in which a desired peptide encoding sequence may be ligated individually into the vector in frame with the lacZ coding region so that a fusion protein is produced; pIN vectors (Inouye & Inouye, 1985; Van Heeke & Schuster, 1989); and the like. pGEX vectors (GE Healthcare, Piscataway, NJ) may also be used to express a desired peptide moiety as a fusion protein with glutathione S-transferase (GST). In general, such fusion proteins are soluble and can easily be purified from lysed cells by adsorption to glutathione-agarose beads, followed by elution in the presence of free glutathione. The pGEX vectors are designed to include thrombin or factor Xa protease cleavage sites so that the cloned desired peptide encoding gene product can be released from the GST moiety.

In an exemplary insect system, Autographa californica nuclear polyhedrosis virus (AcNPV) is used as a vector to express a desired peptide encoding sequence. The virus grows in Spodoptera frugiperda cells. A desired peptide encoding sequence may be cloned individually into a non-essential region (for example the polyhedrin gene) of the virus, and placed under control of an AcNPV promoter (for example the polyhedrin promoter). Successful insertion of a desired peptide encoding sequence will result in inactivation of the polyhedrin gene and production of non-occluded recombinant virus (i.e., virus lacking the proteinaceous coat coded for by the polyhedrin gene). The recombinant viruses are then used to infect Spodoptera frugiperda cells in which the inserted polynucleotide is expressed (see, e.g., Smith et al., 1983; U.S. Pat. No. 4,215,051).

In mammalian host cells, a number of viral-based expression systems may be utilized. In cases where an adenovirus is used as an expression vector, a desired peptide encoding nucleotide sequence may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing desired peptide products in infected hosts (see, e.g., Logan & Shenk, 1984). Specific initiation signals may also be required for efficient translation of inserted desired peptide encoding nucleotide sequences. These signals include the ATG initiation codon and adjacent sequences. In some cases exogenous translational control signals, including, perhaps, the ATG initiation codon, may be provided. Furthermore, the initiation codon should be in phase with the reading frame of the desired peptide encoding coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Nevins, 1986).

In yeast, a number of vectors containing constitutive or inducible promoters may be used. For a review, see, e.g (Bitter et al., 1987; Glover, 1986; Bitter, 1987; Strathern et al., 1981), and (Strathern et al., 1982).

In plants, a variety of different plant expression vectors can be used, and expression of a desired peptide encoding sequence may be driven by any of a number of promoters. For example, viral promoters such as the 35S RNA or 19S RNA promoters of CaMV (Brisson et al., 1984), or the coat protein promoter of TMV (Takamatsu et al., 1987) may be used. Alternatively, plant promoters such as the promoter of the small subunit of RUBISCO (Coruzzi et al., 1984; Broglie et al., 1984), or heat shock promoters, e.g., soybean hsp17.5-E or hsp17.3-B (Gurley et al., 1986) may be used. These constructs can be introduced into plant cells using, for example, Ti plasmids, Ri plasmids, plant virus vectors, direct DNA transformation, microinjection, or electroporation. For reviews of such techniques, see, e.g., Weissbach & Weissbach (Schuler and Zielinski, 1988), and (Grierson & Covey, 1988).

In addition, a host cell strain may be chosen that modulates the expression of the inserted desired peptide encoding sequence, or modifies and processes the desired peptide encoding nucleic acid sequence in a desired fashion. Such modifications (e.g., glycosylation) and processing (e.g., cleavage) of protein products may affect certain functions of the protein. Different host cells have characteristic and specific mechanisms for post-translational processing and modification of proteins and peptides. Appropriate cell lines or host systems can be chosen to ensure the correct or desired modification and processing of the desired protein, polypeptide, or peptide expressed. To this end, eukaryotic host cells that possess the cellular machinery for desired processing of the primary transcript, and glycosylation and/or phosphorylation of desired peptide encoding nucleic acid sequence be used. Such mammalian host cells include, but are not limited to, Chinese hamster ovary (CHO), VERO, baby hamster kidney (BHK), HeLa, monkey kidney (COS), MDCK, 293, 3T3, WI38, human hepatocellular carcinoma (e.g., Hep G2), and U937 cells.

In some embodiments, a recombinant host cell comprising one of the nucleic acid sequences described. In some embodiments, a protein composition comprising one of the polypeptides described.

### II. METHODS OF USE

In some embodiments, molecular engineered variants (some with improved activity) of the described kalium-conducting channelrhodopsin by site-specific mutagenesis and chimera construction. In some embodiments, the channelrhodopsins serve as receptors for phototaxis and the photophobic response. Their photoexcitation initiates depolarization of the cell membrane.

In some embodiments, the rhodopsin domains of several kalium-conducting channelrhodopsins were cloned and determined to have channel activity when they were expressed in mammalian HEK293 cells. Using these methods new potassium-conducting channelrhodopsin variants, were determined to have improved properties with regards to potassium-channelopathies such as those associated with epilepsy, Parkinson's disease, and long-QT syndrome and other cardiac arrhythmias, as well as optogenetics.

One of the major challenges for optogenetic applications, especially in living animals, are scattering of the stimulating light by biological tissues and its absorption by hemoglobin. Optogenetic tools with long-wavelength absorption would exhibit minimal light attenuation from these effects, but most microbial rhodopsins do not fall into this category. For instance, the absorption maximum of ChR2, which possesses several other useful properties and is thereby most frequently used as a depolarizing tool in optogenetics, is 470 nm.

Long-wavelength absorption by optogenetic tools is generally considered desirable to increase the penetration depth of the stimulus light by minimizing tissue scattering and absorption by hemoglobin. In some embodiments, the long-wavelength sensitivity of optogenetic microbial rhodopsins is enhanced using 3,4-Dehydroretinal (A2 retinal). A2 retinal (3,4-dehydroretinal) is a natural retinoid, its 11-cis form being found in photoreceptor cells of certain invertebrates, fish and amphibians, where it may constitute the only retinal, or an additional chromophore to A1 retinal. The presence of an additional double bond in the β-ionone ring of the chromophore results in pigments that absorb light at longer wavelengths, as compared to those formed with A1 (regular) retinal. Variations in A1/A2 ratio cause natural adaptive tuning of spectral sensitivity of vision in the organisms during adaptation to external conditions. Reconstitution of bleached microbial rhodopsins (bacteriorhodopsin, halorhodopsin, sensory rhodopsins I and II) in vitro with all-trans 3,4-dehydroretinal (A2 retinal) also shifts their absorption spectra to longer wavelengths. In some embodiments, spectral properties of optogenetic tools were modified by incorporation of all-trans A2 retinal. The addition of A2 retinal, both ion pumps and channelrhodopsins form functional pigments with significantly red-shifted absorption.

In some embodiments, the long-wavelength sensitivity of optogenetic microbial rhodopsins is enhanced using A2 retinal. In some embodiments, chromophore substitution provides a complementary strategy to improve the efficiency of optogenetic tools. Substitution of A1 retinal by A2 retinal significantly shifts the spectral sensitivity of tested rhodopsins to longer wavelengths typically without altering other aspects of their function.

Optogenetic techniques involve the introduction of light-activated channels and enzymes that allow manipulation of neural activity and control of neuronal function. Thus, in some embodiments, the disclosed methods and compositions can be introduced into cells and facilitate the manipulation of the cells activity and function. See, for example, US publication 20130090454 of U.S. application Ser. No. 13/622,809, as well as, M9attis et al., 2012); and (Zhang et al., 2011).

Optogenetic techniques, and thus the disclosed methods and compositions, can be used to characterize the functions of complex neural circuits and information processing in the normal brain and during various neurological conditions; functionally map the cerebral cortex; characterize and manipulate the process of learning and memory; characterize and manipulate the process of synaptic transmission and plasticity; provide light-controlled induction of gene expression; provide optical control of cell motility and other activities.

Clinical applications of the disclosed methods and compositions include (but are not limited to) potassium channelopathies such as those associated with epilepsy, Parkinson's disease, and long-QT syndrome and other cardiac arrhythmias as well as optogenetic approaches to therapy such as: restoration of vision by introduction of channelrhodopsins in post-receptor neurons in the retina for ocular disorder gene-therapy treatment of age-dependent macular degeneration, diabetic retinopathy, and retinitis pigmentosa, as well as other conditions which result in loss of photoreceptor cells; control of cardiac function by using channelrhodopsins incorporated into excitable cardiac muscle cells in the atrioventricular bundle (bundle of His) to control heart beat rhythm rather than an electrical pacemaker device; restoration of dopamine-related movement dysfunction in Parkinsonian patients; amelioration of depression; recovery of breathing after spinal cord injury; provide noninvasive control of stem cell differentiation and assess specific contributions of transplanted cells to tissue and network function. Any group of electrically active cells may be amenable to KCR suppression, including, but not limited to those listed above and cardiomyocytes. Such KCRs are also potentially useful for efficient photoinhibition of cardiomyocyte action potentials thereby enabling treatment of cardiac dysfunctions including, but not limited to, tachycardia. In some embodiments, the presently described compositions and methods can be used to facilitate optical stimulation of cardiac cells and tissues, without negative electrophysiological effects of current cardiac anti-arrhythmia therapies and alleviate symptoms by stimulating or silencing specific regions with abnormal excitation in the heart or the brain. In some embodiments, such optogenetic-based techniques could be used to silence, restore or reset irregular heartbeat in patients some of which now receive implantable devices.

In some embodiments, the presently described compositions and methods can be used to influence cardiac cells or regions by either direct viral gene delivery (such as but not limited to AAV) or by delivery of KCR-carrying donor cells (such as but not limited to cardiomyocytes, Purkinje and His bundle cells, etc.) generated or transformed in culture.

In some embodiments, a method of membrane hyperpolarization of a cell in a subject suffering from a neuron mediated disorder. said method comprising: delivering to the cell of said subject an expression vector comprising a polynucleotide that encodes an amino acid sequence selected from the group consisting of SEQ ID NO: 1 or SEQ ID NO: 2, which encodes a rhodopsin domain of a kalium-conducting channelrhodopsin expressible in said cell; and expressing said vector in said cell, wherein the expression of the rhodopsin results in membrane hyper depolarization.

In some embodiments, a method of neuronal silencing in a subject suffering from a neuron mediated disorder, said method comprising: delivering to a target neuron of said subject an expression vector comprising a polynucleotide that encodes an amino acid sequence selected from the group consisting of SEQ ID NO: 1 or SEQ ID NO: 2, which encodes a rhodopsin domain of a kalium-conducting channelrhodopsin expressible in said target neuron; and expressing said vector in said target neuron, wherein the expression of the rhodopsin results in silencing of the signal from the target neuron.

In some embodiments, a method of restoring photosensitivity to a retina of a subject suffering from vision loss or blindness, said method comprising: delivering to the retina of said subject an expression vector comprising a polynucleotide that encodes an amino acid sequence selected from the group consisting of SEQ ID NO: 2 or SEQ ID NO: 4 which encodes a humanized rhodopsin domain of an kalium-conducting channelrhodopsin expressible in a retinal neuron; and expressing said vector in said retinal neuron, wherein the expressed rhodopsin renders a high level of membrane potential in said retinal neuron.

Therefore, in some embodiments a kalium-conducting channelrhodopsin, light-gated kalium channels that provide membrane hyper depolarization is provided and may be used to enhance optogenetic techniques and optogenetic approaches to therapy. Kalium-conducting channelrhodopsins, functional or active portions thereof, such as but not limited to the rhodopsin domain, and functional equivalents include, but are not limited to, naturally occurring versions of KCR and those that are orthologs and homologs, and mutant versions of KCR, whether naturally occurring or engineered (by site directed mutagenesis, gene shuffling, or directed evolution, as described in, for example, U.S. Pat. No. 5,837,458). Also included are the use of degenerate nucleic acid variants (due to the redundancy of the genetic code) of the disclosed algae KCR derived polynucleotide sequences.

In some embodiments, are methods of treating a neuronal disorder, comprising: (a) delivering to a target neuron a nucleic acid expression vector that encodes a rhodopsin domain of a kalium-conducting channelrhodopsin, expressible in said target neuron, said vector comprising an open reading frame encoding the rhodopsin domain of an kalium-conducting channelrhodopsin, operatively linked to a promoter sequence, and optionally, a transcriptional regulatory sequence; and (b) expressing said vector in said target neuron, wherein the expressed rhodopsin that results in membrane hyperpolarization and neuronal silencing of said target neuron upon exposure to light. In some embodiments, the rhodopsin domain is encoded by SEQ ID NO: 3, SEQ ID NO: 4, SEQ ID NO: 5 or SEQ ID NO: 6.

In some embodiments, are methods of treating a neuronal disorder, comprising: (a) delivering to a target neuron a nucleic acid expression vector that encodes a rhodopsin domain of a kalium-conducting channelrhodopsin, expressible in said target neuron, said vector comprising an open reading frame encoding the rhodopsin domain of an kalium-conducting channelrhodopsin, operatively linked to a promoter sequence, and optionally, a transcriptional regulatory sequence; and (b) expressing said vector in said target neuron, wherein the expressed rhodopsin silences said target neuron upon exposure to light. In some embodiments, the rhodopsin domain is encoded by SEQ ID NO: 1 or SEQ ID NO: 2.

In some embodiments, are methods of restoring light sensitivity to a retina, comprising: (a) delivering to a retinal neuron a nucleic acid expression vector that encodes a rhodopsin domain of a kalium-conducting channelrhodopsin, expressible in the retinal neuron; said vector comprising an open reading frame encoding the rhodopsin domain of a kalium-conducting channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring light sensitivity to said retina or a portion thereof. In some embodiments, the rhodopsin domain is encoded by SEQ ID NO: 1 or SEQ ID NO: 2.

In some embodiments, are methods of restoring light sensitivity to a retina, comprising: (a) delivering to a retinal neuron a nucleic acid expression vector that encodes a rhodopsin domain of a kalium-conducting channelrhodopsin, expressible in the retinal neuron; said vector comprising an open reading frame encoding the rhodopsin domain of a kalium-conducting channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring light sensitivity to said retina or a portion thereof. In some embodiments, the rhodopsin domain is encoded by SEQ ID NO: 3, SEQ ID NO: 4, SEQ ID NO: 5 or SEQ ID NO: 6

In some embodiments, are methods of restoring photosensitivity to a retina of a subject suffering from vision loss or blindness in whom retinal photoreceptor cells are degenerating or have degenerated and died, said method comprising: (a) delivering to the retina of said subject a nucleic acid vector that encodes a rhodopsin domain of a kalium-conducting channelrhodopsin expressible in a retinal neuron; said vector comprising an open reading frame encoding the rhodopsin domain of a kalium-conducting channelrhodopsin operatively linked to a promoter sequence, and optionally, a transcriptional regulatory sequence; and (b) expressing said vector in said retinal neuron, wherein the expressed rhodopsin renders said retinal neuron photosensitive, thereby restoring photosensitivity to said retina or a portion thereof. In some embodiments, the rhodopsin domain is encoded by SEQ ID NO: 1 or SEQ ID NO: 2.

In some embodiments, the presently described compositions and methods can be used to facilitate optical stimulation of cardiac cells and tissues, without negative electrophysiological effects of current cardiac anti-arrhythmia therapies and alleviate symptoms by stimulating or silencing specific regions with abnormal excitation in the heart or the brain. In some embodiments, such optogenetic based techniques could be used to silence, restore or reset irregular heartbeat in patients some of which now receive implantable devices.

In some embodiments, the presently described compositions and methods can be used to influence cardiac cells or regions by either direct viral gene delivery (such as but not limited to AAV) or by delivery of KCR-carrying donor cells (such as but not limited to cardiomyocytes, Purkinje and His bundle cells, etc.) generated or transformed in culture.

In some embodiments is a method of treating a disorder in an electrically active cell in a subject suffering from a disorder that involves electrically active cells, said method comprising: (a) delivering to the cell of said subject an expression vector comprising a polynucleotide that encodes an amino acid sequence selected from SEQ ID NO: 1 or SEQ ID NO: 2 expressible in said cell; and (b) expressing said vector in said electrically active cell, wherein the expressed rhodopsin silences the signal from said electrically active cell.

In some embodiments is a method of treating a disorder in an electrically active cell in a subject suffering from a disorder that involves electrically active cells, said method comprising: (a) delivering to said subject a transgenic cell comprising an expression vector comprising a polynucleotide that encodes an amino acid sequence selected from SEQ ID NO: 1 or SEQ ID NO: 2 expressible in said transgenic cell; and (b) expressing said vector in said transgenic cell, wherein the expression silences the signal from an electrically active cell.

In some embodiments is a method of silencing an electrically active cell in a subject suffering from an electrically active cell mediated disorder, said method comprising: (a) delivering to a target neuron of said subject an expression vector comprising a polynucleotide that encodes an amino acid sequence selected from SEQ ID NO: 1 or SEQ ID NO: 2 which encodes a rhodopsin domain of a kalium-conducting channelrhodopsin expressible in said target neuron; and (b) expressing said vector in said target electrically active cell, wherein the expressed rhodopsin results in silencing of the signal from the electrically active cell. In some embodiments is a recombinant host cell, wherein said host cell is an isolated an electrically active cell.

In some embodiments, a method of treating a neuronal disorder comprises: (a) delivering to a target neuron a nucleic acid expression vector that encodes a rhodopsin domain of a kalium-conducting channelrhodopsin, expressible in said target neuron; said vector comprising an open reading frame encoding the rhodopsin domain of a kalium-conducting channelrhodopsin operatively linked to a promoter sequence, and optionally, transcriptional regulatory sequences; and (b) expressing the expression vector in the target neuron, wherein the expressed kalium-conducting channelrhodopsin silences the target neuron upon exposure to light. In some embodiments an above-described expression vector also comprises one or more transcriptional regulatory sequences operably linked to the promoter and rhodopsin domain sequences. In some embodiments, the rhodopsin domain of a kalium-conducting channelrhodopsin has the amino acid sequence of all or part of SEQ ID NO: 1 or SEQ ID NO: 2 and the rhodopsin domain sequences of SEQ ID NO: 1 or SEQ ID NO: 2, or a biologically active fragment thereof that retains the biological activity of the encoded rhodopsin domain of a channelrhodopsin or is a biologically active conservative amino acid substitution variant of SEQ ID NO: 1 or SEQ ID NO: 2 or of said fragment. In some embodiments, the expression vector comprises an AAV viral vector. In some embodiments, the promoter is a constitutive promoter. In some embodiments, the constitutive promoter is a CMV promoter or a hybrid CMV enhancer/chicken β-actin (CAG) promoter. In some embodiments, the promoter is an inducible and/or a cell type-specific promoter.

In some embodiments, a method of restoring light sensitivity to a retina comprises (a) delivering to a retinal neuron in a subject a nucleic acid expression vector that encodes a rhodopsin domain of a kalium-conducting channelrhodopsin, expressible in the retinal neuron; said expression vector comprising an open reading frame encoding the rhodopsin domain of a kalium-conducting channelrhodopsin operatively linked to a promoter sequence, and optionally, one or more transcriptional regulatory sequences; and (b) expressing the expression vector in the retinal neuron, wherein the expressed rhodopsin renders the retinal neuron photosensitive, thereby restoring light sensitivity to the retina or a portion thereof. In some embodiments, the rhodopsin domain of the kalium-conducting channelrhodopsin has the amino acid sequence of all or part of SEQ ID NO: 1 or SEQ ID NO: 2, or a biologically active fragment thereof that retains the biological activity of the encoded rhodopsin domain of a kalium-conducting channelrhodopsin or is a biologically active conservative amino acid substitution variant of SEQ ID NO: 1 or SEQ ID NO: 2, or of said fragment. In some embodiments, the expression vector comprises an AAV (e.g., AAV2) viral vector. In some embodiments, the promoter is a constitutive promoter. In some embodiments, the constitutive promoter is a CMV promoter or a hybrid CMV enhancer/chicken β-actin (CAG) promoter. In some embodiments, the promoter is an inducible and/or a cell type-specific promoter.

In some embodiments, a method of restoring photosensitivity to a retina of a subject suffering from vision loss or blindness in whom retinal photoreceptor cells are degenerating or have degenerated and died comprises: (a) delivering to the retina of the subject a nucleic acid expression vector that encodes a rhodopsin domain of a kalium-conducting channelrhodopsin expressible in retinal neurons; said expression vector comprising an open reading frame encoding the rhodopsin domain of a kalium-conducting channelrhodopsin operatively linked to a promoter sequence, and optionally, transcriptional regulatory sequences; and (b) expressing the expression vector in the retinal neuron, wherein the expression of the rhodopsin renders the retinal neuron photosensitive, thereby restoring photosensitivity to said retina or a portion thereof. In some embodiments, the rhodopsin domain of a kalium-conducting channelrhodopsin has the amino acid sequence of all or part of SEQ ID NO: 1 or SEQ ID NO: 2 or the rhodopsin domain sequences of SEQ ID NO: 1 or SEQ ID NO: 2, or a biologically active fragment thereof that retains the biological activity of the encoded rhodopsin domain of a kalium-conducting channelrhodopsin or is a biologically active conservative amino acid substitution variant SEQ ID NO: 1 or SEQ ID NO: 2, or of said fragment. In some embodiments, the expression vector comprises an AAV (e.g., AAV2) viral vector. In some embodiments, the promoter is a constitutive promoter. In some embodiments, the constitutive promoter is a CMV promoter or a hybrid CMV enhancer/chicken β-actin (CAG) promoter. In other embodiments, the promoter is an inducible and/or a cell type-specific promoter.

A. Compositions as Therapeutics

The use of channelrhodopsins, or active fragments thereof such as but not limited to the rhodopsin domain as therapeutics. In certain embodiments the presently disclosed compositions and are used to improve optogenetic techniques and applications as well as can be used to aid in diagnosis, prevention, and/or treatment of among other things neuron mediated disorders, neurologic disorders (such as, but not limited to epilepsy, Parkinson's disease, and long-QT syndrome and other cardiac arrhythmias) and ocular disorders.

In certain embodiments the presently disclosed compositions can be administered in combination with one or more additional compounds or agents (“additional active agents”) for the treatment, management, and/or prevention of among other things neuron mediated disorders, neurologic disorders (such as, but not limited to epilepsy, Parkinson's disease, and long-QT syndrome and other cardiac arrhythmias) and ocular disorders. Such therapies can be administered to a patient at therapeutically effective doses to treat or ameliorate, among other things, neuron mediated disorders, neurologic disorders (such as, but not limited to epilepsy, Parkinson's disease, and long-QT syndrome and other cardiac arrhythmias) and ocular disorders. A therapeutically effective dose refers to that amount of the compound sufficient to result in any delay in onset, amelioration, or retardation of disease symptoms.

Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, expressed as the ratio LD₅₀/ED₅₀. Compositions that exhibit large therapeutic indices are preferred. Compounds that exhibit toxic side effects may be used in certain embodiments, however, care should usually be taken to design delivery systems that target such compositions preferentially to the site of affected tissue, in order to minimize potential damage to uninfected cells and, thereby, reduce side effects.

Data obtained from cell culture assays and animal studies can be used in formulating a range of dosages for use in humans. The dosages of such compositions lie preferably within a range of circulating concentrations that include the ED₅₀ with little or no toxicity. The dosage may vary within this range depending on the dosage form employed and the route of administration utilized. For any composition, the therapeutically effective dose can be estimated initially from cell culture assays. A dose may be formulated in animal models to achieve a circulating plasma concentration range that includes the IC₅₀ (i.e., the concentration of the test composition that achieves a half-maximal inhibition of symptoms) as determined in cell culture. Such information can be used to more accurately determine useful doses in humans. Plasma levels may be measured, for example, by high performance liquid chromatography.

When the therapeutic treatment of among other things neurologic disorders (such as Parkinson's disease) and as therapy for ocular disorders is contemplated, the appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human studies of efficacy are undertaken, Phase I clinical studies help establish safe doses.

Additionally, the bioactive agent may be coupled or complexed with a variety of well-established compositions or structures that, for instance, enhance the stability of the bioactive agent, or otherwise enhance its pharmacological properties (e.g., increase in vivo half-life, reduce toxicity, etc.).

Such therapeutic agents can be administered by any number of methods known to those of ordinary skill in the art including, but not limited to, inhalation, subcutaneous (sub-q), intravenous (I.V.), intraperitoneal (I.P.), intramuscular (I.M.), or intrathecal injection, or topically applied (transderm, ointments, creams, salves, eye drops, and the like), as described in greater detail below.

B. Pharmaceutical Compositions

Pharmaceutical compositions for use in accordance with the presently described compositions may be formulated in conventional manners using one or more physiologically acceptable carriers or excipients.

The pharmaceutical compositions can comprise formulation materials for modifying, maintaining, or preserving, for example, the pH, osmolarity, viscosity, clarity, color, isotonicity, odor, sterility, stability, rate of dissolution or release, adsorption or penetration of the composition. Suitable formulation materials include, but are not limited to: amino acids (for example, glycine, glutamine, asparagine, arginine and lysine); antimicrobials; antioxidants (for example, ascorbic acid, sodium sulfite and sodium hydrogen-sulfite); buffers (for example, borate, bicarbonate, Tris-HCl, citrates, phosphates and other organic acids); bulking agents (for example, mannitol and glycine); chelating agents (for example, ethylenediamine tetraacetic acid (EDTA)); complexing agents (for example, caffeine, polyvinylpyrrolidone, beta-cyclodextrin, and hydroxypropyl-beta-cyclodextrin); fillers; monosaccharides, disaccharides, and other carbohydrates (for example, glucose, mannose and dextrins); proteins (for example, serum albumin, gelatin and immunoglobulins); coloring, flavoring, and diluting agents; emulsifying agents; hydrophilic polymers (for example, polyvinylpyrrolidone); low molecular weight polypeptides; salt-forming counterions (for example, sodium); preservatives (for example, benzalkonium chloride, benzoic acid, salicylic acid, thimerosal, phenethyl alcohol, methylparaben, propylparaben, chlorhexidine, sorbic acid and hydrogen peroxide); solvents (for example, glycerin, propylene glycol and polyethylene glycol); sugar alcohols (for example, mannitol and sorbitol); suspending agents; surfactants or wetting agents (for example, pluronics, PEG, sorbitan esters, polysorbates (for example, polysorbate 20 and polysorbate 80), triton, tromethamine, lecithin, cholesterol, and tyloxapal); stability enhancing agents (for example, sucrose and sorbitol); tonicity enhancing agents (for example, alkali metal halides (for example, sodium or potassium chloride), mannitol, and sorbitol); delivery vehicles; diluents; excipients; and pharmaceutical adjuvants (Gennaro, 1990).

Additionally, the described therapeutic peptides can be linked to a half-life extending vehicle. Certain exemplary half-life extending vehicles are known in the art, and include, but are not limited to, the Fc domain, polyethylene glycol, and dextran (see, e.g., PCT Patent Application Publication No. WO 99/25044).

These agents may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The agents may also be formulated as compositions for rectal administration such as suppositories or retention enemas, e.g., containing conventional suppository bases such as cocoa butter or other glycerides.

In addition to the formulations described previously, the agents may also be formulated as a depot preparation. Such long-acting formulations may be administered by implantation (for example subcutaneously or intramuscularly) or by intramuscular injection. For example, agents may be formulated with suitable polymeric or hydrophobic materials (for example as an emulsion in an acceptable oil), ion exchange resins, or as sparingly soluble derivatives, for example, as a sparingly soluble salt. The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

Active compositions can be administered by controlled release means or by delivery devices that are well-known to those of ordinary skill in the art. Examples include, but are not limited to, those described in U.S. Pat. Nos. 3,845,770, 3,916,899, 3,536,809, 3,598,123, 4,008,719, 5,674,533, 5,059,595, 5,591,767, 5,120,548, 5,073,543, 5,639,476, 5,354,556, and 5,733,566. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof, to provide the desired release profile in varying proportions. Exemplary sustained release matrices include, but are not limited to, polyesters, hydrogels, polylactides (see, e.g., U.S. Pat. No. 3,773,919 and European Patent Application Publication No. EP 058,481), copolymers of L-glutamic acid and gamma ethyl-L-glutamate (see, e.g., Sidman et al., 1983), poly (2-hydroxyethyl-methacrylate) (see, e.g., Langer et al., 1981, and Langer, 1982), ethylene vinyl acetate (Langer et al., 1981), and poly-D(−)-3-hydroxybutyric acid (European Patent Application Publication No. EP 133,988). Sustained release compositions may include liposomes, which can be prepared by any of several methods known in the art (see, e.g., Eppstein et al., 1985, and European Patent Application Publication Nos. EP 036,676, EP 088,046, and EP 143,949). Suitable controlled-release formulations known to those of ordinary skill in the art, including those described herein, can be readily selected for use with the presently disclosed compositions. Certain embodiments encompass single unit dosage forms suitable for oral administration such as, but not limited to, tablets, capsules, gelcaps, and caplets that are adapted for controlled-release.

All controlled-release pharmaceutical products have a common goal of improving therapy over that achieved by their non-controlled counterparts. Ideally, use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of active ingredient that promptly produces the desired therapeutic effect, and gradually and continually release other amounts of active ingredient to maintain this level of therapeutic or prophylactic effect over an extended period of time. In order to maintain this relatively constant level of active ingredient in the body, the drug must be released from the dosage form at a rate that will replace the amount of active ingredient being metabolized and excreted from the body. Controlled-release of an active ingredient can be stimulated by various conditions including, but not limited to, pH, temperature, enzymes, water, or other physiological conditions or compositions.

In some cases, active ingredients of the disclosed methods and compositions are preferably not administered to a patient at the same time or by the same route of administration. Therefore, in some embodiments are kits that, when used by the medical practitioner, can simplify the administration of appropriate amounts of active ingredients to a patient.

A typical kit comprises a single unit dosage form of one or more of the therapeutic agents disclosed, alone or in combination with a single unit dosage form of another agent that may be used in combination with the disclosed compositions. Disclosed kits can further comprise devices that are used to administer the active ingredients. Examples of such devices include, but are not limited to, syringes, drip bags, patches, and inhalers.

Disclosed kits can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. However, in specific embodiments, the disclosed formulations do not contain any alcohols or other co-solvents, oils or proteins.

C. Transgenic Animals

The present disclosure provides methods and compositions for the creation and use of both human and non-human transgenic animals that carry an algae derived kalium-conducting channelrhodopsin transgene in all their cells, as well as non-human transgenic animals that carry an algae derived kalium-conducting channelrhodopsin transgene in some, but not all their cells, for example in certain electrically active cells. Human and non-human mammals of any species, including, but not limited to, mice, rats, rabbits, guinea pigs, pigs, micro-pigs, goats, and non-human primates, e.g., baboons, monkeys, and chimpanzees, can be used to generate transgenic animals carrying an algae derived kalium-conducting channelrhodopsin polynucleotide (and/or expressing an algae derived polypeptide) may be integrated as a single transgene or in concatamers, e.g., head-to-head or head-to-tail tandems. An algae-derived kalium-conducting channelrhodopsin transgene may also be selectively introduced into and activated in a particular cell-type (see, e.g., Lakso et al., 1992). The regulatory sequences required for such a cell-type specific activation will depend upon the particular cell-type of interest, and will be apparent to those of skill in the art.

Should it be desired that an algae-derived kalium-conducting channelrhodopsin, or fragment thereof, transgene be integrated into the chromosomal site of the endogenous copy of the mammalian potassium-conducting channelrhodopsin gene, gene targeting is generally preferred. Briefly, when such a technique is to be utilized, vectors containing some nucleotide sequences homologous to the endogenous kalium-conducting channelrhodopsin gene are designed for the purpose of integrating, via homologous recombination with chromosomal sequences, into and disrupting the function of the endogenous channelrhodopsin gene (i.e., “knock-out” animals). In this way, the expression of the endogenous channelrhodopsin gene may also be eliminated by inserting non-functional sequences into the endogenous channelrhodopsin gene. The transgene may also be selectively introduced into a particular cell-type, thus inactivating the endogenous channelrhodopsin gene in only that cell-type (see, e.g., Gu et al., 1994). The regulatory sequences required for such a cell-type specific inactivation will depend upon the particular cell-type of interest, and will be apparent to those of skill in the art.

Any technique known in the art may be used to introduce a channelrhodopsin, or fragment thereof, transgene into animals to produce the founder lines of transgenic animals. Such techniques include but are not limited to: pronuclear microinjection (U.S. Pat. No. 4,873,191); retrovirus-mediated gene transfer into germ lines (van der Putten et al., 1985); gene targeting in embryonic stem cells (Thompson et al., 1989); electroporation of embryos (Lo, 1983); sperm-mediated gene transfer (Lavitrano et al., 1989); and positive-negative selection, as described in U.S. Pat. No. 5,464,764. For a review of such techniques, see, e.g., Gordon, 1989.

Once transgenic animals have been generated, the expression of the recombinant channelrhodopsin gene, or fragment thereof, may be assayed utilizing standard techniques. Initial screening may be accomplished by Southern blot analysis or PCR techniques to analyze animal tissues to assay whether integration of the channelrhodopsin transgene has taken place. The level of mRNA expression of the channelrhodopsin transgene in the tissues of the transgenic animals may also be assessed using techniques that include, but are not limited to, Northern blot analysis of cell-type samples obtained from the animal, in situ hybridization analysis, and RT-PCR. Samples of an algae derived channelrhodopsin-expressing tissue can also be evaluated immunocytochemically using antibodies selective for the channelrhodopsin transgene product.

D. Transgene Based Therapies

In certain embodiments the presently disclosed compositions and are used to improve optogenetic techniques and applications as well as can be used to aid in diagnosis, prevention, and/or treatment of neurologic disorders, (such as, but not limited to epilepsy, Parkinson's disease, and long-QT syndrome and other cardiac arrhythmias) and ocular disorders.

In some embodiments, methods and compositions are used to identify and characterize multiple channelrhodopsins derived from algae. The cloning and expression of the rhodopsin domain of the channelrhodopsins and expression in mammalian cells demonstrates that these channelrhodopsins have improved characteristics that can be used for optogenetic applications as well as therapeutic agents.

For example, a disclosed method and composition may be used in, among other things, retinal gene therapy for mammals (as described in, among others, U.S. Pat. Nos. 5,827,702, 7,824,869 and US Patent Publication Number 20100015095 as well as in WIPO publications WO 2000/15822 and WO 1998/48097). A genetically engineered ocular cell is produced by contacting the cell with an exogenous nucleic acid under conditions in which the exogenous nucleic acid is taken up by the cell for expression. The exogenous nucleic acid is described as a retrovirus, an adenovirus, an adeno-associated virus or a plasmid. Retinal gene transfer of a reporter gene, green fluorescent protein (GFP), using a recombinant adeno-associated virus (rAAV) was demonstrated in normal primates (Bennett et al., 1999). The rescue of photoreceptors using gene therapy in a model of rapid degeneration of photoreceptors using mutations of the RP65 gene and replacement therapy with the normal gene to replace or supplant the mutant gene (See, for example, US Patent Publication 2004/0022766) has been used to treat a naturally-occurring dog model of severe disease of retinal degenerations—the RPE65 mutant dog, which is analogous to human LCA. By expressing photosensitive membrane-channels or molecules in surviving retinal neurons of the diseased retina by viral based gene therapy method, the present invention may produce permanent treatment of the vision loss or blindness with high spatial and temporal resolution for the restored vision.

The nucleic acids sequences that encode an active portion of the presently disclosed kalium-conducting channelrhodopsins, include but are not limited to the nucleic acid sequences that encode the rhodopsin domains identified in SEQ ID NO: 1 or SEQ ID NO: 2 or the optimized rhodopsin domain sequences of SEQ ID NO: 3, SEQ ID NO: 4, SEQ ID NO: 5 or SEQ ID NO: 6.

In some embodiments, introduction and expression of channelrhodopsins, such as those described herein, in ocular neuronal cells, for example, impart light sensitivity to such retinas and restoring one or more aspects of visual responses and functional vision to a subject suffering from such degeneration. By restoring light sensitivity to a retina lacking this capacity, due to disease, a mechanism for the most basic light-responses that are required for vision is provided. In some embodiments, the functional domains of kalium-conducting channelrhodopsins may be used to restore light sensitivity to the retinas that have undergone rod and cone degeneration by expressing the channelrhodopsin in inner retinal neurons in vivo. In some embodiments these channelrhodopsins may be introduced using techniques that include, but are not limited to, retinal implants, cortical implants, lateral geniculate nucleus implants, or optic nerve implants.

In some embodiments, the kalium-conducting channelrhodopsins are inserted into the retinal neurons that survived after the rods and cones have died in an area or portion of the retina of a subject, using the transfer of nucleic acids, alone or within an expression vector. Such expression vectors may be constructed, for example, by introduction of the desired nucleic acid sequence into a virus system known to be of use for gene therapy applications, such as, but not limited to, AAV (e.g., AAV2), retroviruses and alike.

In some embodiments the kalium-conducting channelrhodopsins may be inserted into retinal interneurons. These cells then can become light sensitive and send signals via the optic nerve and higher order visual pathways to the visual cortex where visual perception occurs, as has been demonstrated electrophysiologicly in mice. In some embodiments, among other routes, intravitreal and/or subretinal injections may be used to deliver channelrhodopsin molecules or virus vectors expressing the same.

In some embodiments, the active portion of the presently disclosed algal derived kalium-conducting channelrhodopsins, such as but not limited to the rhodopsin domain of these kalium-conducting channelrhodopsins, can be used to restore light sensitivity to a retina, by delivering to retinal neurons a nucleic acid expression vector that encodes algal derived kalium-conducting channelrhodopsins (such as but not limited to the rhodopsin domain of these kalium-conducting channelrhodopsins) that is expressible in the neurons, which vector comprises an open reading frame encoding the rhodopsin, and operatively linked thereto, a promoter sequence, and optionally, transcriptional regulatory sequences; and expressing the vector in the neurons, thereby restoring light sensitivity.

In certain embodiments the channel rhodopsin can be algal derived kalium-conducting channelrhodopsins such as, but not limited to functional domains of kalium-conducting channelrhodopsins, such as, but not limited to, SEQ ID NO: 1 or SEQ ID NO: 2 or a biologically active fragment or conservative amino acid substitution variant thereof, such as but not limited to the rhodopsin domain. The vector system may be recombinant AAV (e.g., AAV2), the promoter may be a constitutive promoter such as, but not limited to, a CMV promoter or a hybrid CMV enhancer/chicken β-actin promoter (CAG).

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Bioinformatics and molecular biology. Initially, the predicted protein sequences encoded by the genes Hypho2016_00006030 and Hypho2016 00006031 (395 and 383 amino acid residues, respectively) were obtained from the database provided by reference16 (world-wide-web at ebi.ac.uk/biostudies/studies/S-BSST46), the file hyphochytrium_catenoides_predicted_proteins_renamed_modified.fasta. However, a large part of TM6 was missing from the Hypho2016_00006030 prediction. Therefore, the inventors corrected its TM domain by performing TBLASTN search of the WGS data for H. catenoides strain ATCC 18719 (accession numbers FLMG00000000.1 and CAFC00000000.2) at the National Center for Biotechnology Information (NCBI), using the sequence provided16 as a query. The resultant alignment allowed us to recover the full sequence (401 residues). DNA polynucleotides encoding the corrected transmembrane domains (residues 1-265) of Hypho2016_00006030 (HcKCR2) and Hypho2016_00006031 (HcKCR1) were optimized for human codon usage and synthesized at Genscript Biotech Corporation. The truncation points (20 residues after the end of helix 7) were determined by prediction of protein topology using the TMHMM 2.0 server43. The sequence information was deposited to GenBank (accession numbers MZ826861 and MZ826862, respectively).

Rhodopsin sequences were aligned using MUSCLE as implemented in MegAlign Pro software v. 17.1.1 (DNASTAR Lasergene) with default parameters. Phylogeny was analyzed with IQ-TREE v. 2.1.244 using automatic model selection and ultrafast bootstrap approximation (1000 replicates)45. The best tree was visualized and annotated with iTOL v. 6.346.

For expression in HEK293/HEK293A (human embryonic kidney) cells the polynucleotides encoding the transmembrane domains of HcKCR1 and HcKCR2 were cloned into the mammalian expression vector pcDNA3.1 (Life Technologies) in frame with a C-terminal mCherry tag. For expression in P. pastoris the polynucleotide encoding the transmembrane domain of HcKCR1 was fused with a C-terminal His7 tag and cloned into the pPICZaA vector (Invitrogen). For expression in mouse cortical neurons HcKCR1 and HcKCR2 were tagged with EYFP (enhanced yellow fluorescent protein) at the C-terminus and cloned into the pAAV-CAG vector.

HEK293/HEK293A cell culture and transfection. No cell lines from the list of known misidentified cell lines maintained by the International Cell Line Authentication Committee were used in this study. The HEK293 cells were obtained from the American Type Culture Collection (ATCC; catalog #CRL-1573), and the HEK293A cells, from Thermo Fisher Scientific (catalog #R70507), and grown in high-glucose Dulbecco's Modified Eagle medium supplemented with 5% fetal bovine serum (FBS), 2 mM glutamine, 100 unit/mL penicillin, and 100 μg/mL streptomycin at 37° C. with 5% CO₂. The HEK293 cells were plated on 2-cm diameter plastic dishes 48-72 hrs before experiments, grown for 24 hrs and transfected with 10 μl of ScreenFectA transfection reagent (Waco Chemicals USA, Richmond) using 3 μg DNA per dish. The HEK293A cells were plated on 30-70 kD poly-D-lysine-coated 12-mm circular coverslips (Carolina cover glass #0, 633009) in 24-well plates (P24-1.5H-N, Cellvis) at 30% confluence, and transfected with 1.2 μL, of FuGENE HD transfection reagent (E2311, Promega) using 400 ng DNA per well 48-72 hrs before measurements. Immediately after transfection, all-trans-retinal (Sigma) was added as a stock solution in ethanol at the final concentration of 5 μM.

One-photon (1P) patch clamp electrophysiology. Whole-cell voltage clamp recordings in HEK293 cells were performed with an Axopatch 200B amplifier (Molecular Devices). The signals were digitized with a Digidata 1440A using pClampEx 10.7 software (both from Molecular Devices). Patch pipettes with resistances of 2-5 MΩ were fabricated from borosilicate glass. The composition of all solutions is shown in Supplementary Table 1. A 4 M salt bridge was used in all experiments. All IV dependencies were corrected for liquid junction potentials (LJP) calculated using the ClampEx built-in LJP calculator (Supplementary Table 1). Continuous light pulses were provided by a Polychrome IV light source (T.I.L.L. Photonics GMBH) in combination with a mechanical shutter (Uniblitz Model LS6, Vincent Associates, Rochester; half-opening time 0.5 ms). Maximal quantum density at the focal plane of the 40× objective lens was ˜7 mW mm-2 at 540 nm. The action spectra were constructed by calculation of the initial slope of photocurrent in the linear range of the dependence on the quantum density (<25 μW mm-2), corrected for the quantum density measured at each wavelength and normalized to the maximal value. Desensitization of photocurrents under continuous illumination was quantified as the difference between the peak value and that at the end of a 1-s light pulse, divided by the peak value. Laser excitation was provided by a Minilite II Nd:YAG laser (532 nm, pulsewidth 6 ns, energy 12 mJ; Continuum). Initial analysis of the recorded data was carried out using ClampFit 10.7. The current traces were logarithmically filtered using LogPro software47. Curve fitting was performed by Origin Pro 2016 software (OriginLab Corporation). All measurements were carried out at room temperature (25° C.).

Noise analysis. HcKCR1 photocurrent traces were recorded at −60 mV at room temperature for 20 s in the dark and during a 20-s light pulse of intensity eliciting a half-maximal response with 130 mM KCl in the bath and pipette. The plateau current was fit with a double exponential, and the fit signal was subtracted from the current trace. Power spectra for the light and dark conditions were calculated from the current traces using pClamp software. Three spectra for each condition were calculated for each of five cells and pooled together to obtain the mean light and dark spectra. The difference (mean light minus mean dark spectrum) was fit between 1 Hz and 1 kHz with a Lorentzian function to determine the zero-frequency asymptote S(0) and the corner frequency fc. The unitary conductance (γ) was estimated from these parameters and the amplitude of the whole-cell channel current (I), the holding potential (Vh) and the reversal potential of the channel current (Vr) using the formula: γ=πS(0)fc/2I(Vh−Vr).

Two-photon (2P) patch clamp electrophysiology. Infrared 2P excitation of HcKCRs expressed in HEK293A cells was conducted on an inverted microscope with multi-photon capability (A1R-MP, Nikon Instruments). A coverslip seeded with the transfected cells was placed in a custom glass-bottom chamber based on Chamlide EC (Live Cell Instrument) with a glass bottom made with a 24×24 mm cover glass #1 (Erie Scientific, 89082-270). Cells were continuously perfused with external solution (Supplementary Table 1, the osmolarity of which adjusted to 290 mOsm/L with D-(+)-glucose), at ˜4 mL/min with a peristaltic pump (505DU, Watson Marlow). The osmolarity of the pipette solution (Supplementary Table 1) was adjusted to 280 mOsm/L. Whole-cell voltage clamp recordings were carried out using a MultiClamp 700B amplifier (Molecular Devices). The cells were held at −20 mV, with the command voltage compensated for the 4.4 mV LJP calculated using the ClampEx 11.1 (Molecular Devices) build-in calculator. The signal was digitized with an Axon Digidata 1550B1 Low Noise system with HumSilencer (Molecular Devices), and the current was recorded at 10 kHz using ClampEx 11.1. The65etwees resistance (Ra) and the membrane resistance (Rm) were tested before and after each recording. All measurements were carried out at room temperature (22-23° C.).

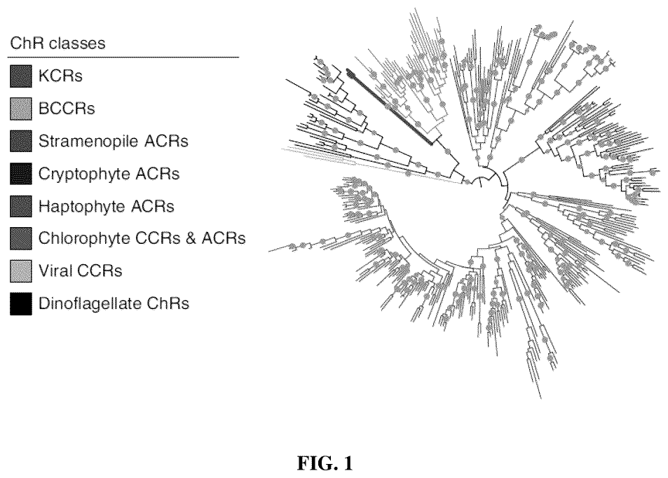

The excitation infrared (NIR) light was generated by a titanium: sapphire femtosecond laser (Chameleon Ultra II, Coherent) with a repetition rate of 80 MHz and a tuning range between 680 and 1080 nm. Laser power was tuned using an acousto-optic modulator and delivered to the sample plane through a 40×0.95-NA objective (CFI Plan Apochromat Lambda, Nikon Instruments). Scanning across XY regions-of-interest was achieved using a pair of galvanometer scanners. Thirty sequential raster scans (total duration ˜1 s) were conducted over a 16×16 μm (64×64 pixels) area with a dwell time of 2.7 I's per pixel and no time gap between each scan of a pulse. The dimensions of the excitation area denoted above were chosen to fit an approximate size of an average HEK293A cell. The jagged appearance of the photocurrent traces (FIG. 1g) is owing to raster scanning artifacts such as laser being turned off when flying back to the starting pixel after each scan. The interval between pulses was ˜60 s to avoid KCR desensitization. The absence of significant desensitization was confirmed by a <10% decrease in the peak photocurrent 65etweenn light pulses at the same power and wavelength (3.5 mW, 1040 nm for HcKCR1, and 4.8 mW, 960 nm for HcKCR2) between the start and end of each recording.

To quantify the power dependence of 2P activation of HcKCRs, 2P illumination at the approximate peak wavelength was used (1040 and 960 nm for HcKCR1 and HcKCR2, respectively). The excitation power at the sample plane was varied between ˜0.1 and ˜6 mW, as measured by a microscope slide power sensor (S170C, Thorlabs). To determine the two-photon action spectra of HcKCRs, the excitation wavelength was varied from 800 to 1080 nm in 40-nm increments. At each wavelength, the laser was tuned to ˜3.5 mW at the sample plane (in the linear range of the power dependence). The maximal deviation from the mean power at the sample plane per recording was <10%. The 2P action spectra were constructed by measuring the initial linear slope of the photocurrent rise at each wavelength. The obtained values were then corrected for the exact power measured at each wavelength. The data points were connected with a spline line using Origin Pro 2016 software (OriginLab Corporation) and normalized to the maximal value of the spline curve.

Purification of HcKCR1 from Pichia pastoris. The HcKCR1-7His-pPICZaA plasmid was linearized with SacI and delivered into P. pastoris SMD1168 by electroporation. A single colony resistant to 0.5 mg/ml zeocin was picked and inoculated into buffered complex glycerol medium, after which the cells were transferred to buffered complex methanol (0.5%) medium supplemented with 5 μM all-trans-retinal (Sigma-Aldrich) and grown at 30° C. with shaking at 200 rpm. After 24 h, the cells were harvested and disrupted in 100 ml of ice-cold buffer A (20 mM Hepes, pH 7.4, 150 mM NaCl, 1 mM EDTA, 5% glycerol) using a bead beater. Cell debris was removed by centrifugation at 5,000×g for 10 min. Membrane fragments were collected by ultracentrifugation at 190,000×g for 1 h, and then solubilized in 20 ml of buffer B (20 mM Hepes, pH 7.5, 350 mM NaCl, 5% glycerol) and 1% dodecyl-β-D-maltopyranoside (DDM) at 4° C. for 1 h. Non-solubilized material was removed by ultracentrifugation at 110,000×g for 1 h. The supernatant was mixed with nickel-nitrilotriacetic acid resin (Qiagen) with 15 mM imidazole and incubated at 4° C. for 1 h. After washing the resin with buffer B containing 0.02% DDM and 40 mM imidazole, the protein was eluted with buffer B containing 0.02% DDM and 300 mM imidazole, concentrated using Amicon® Ultra centrifugal filters (Millipore) at 4° C., and washed with buffer B containing 0.02% DDM to remove imidazole.

UV-visual absorption spectroscopy and flash photolysis. Absorption spectra of purified HcKCR1 were recorded using a Cary 4000 spectrophotometer (Varian). Light-induced absorption changes were measured with a laboratory-constructed crossbeam apparatus. Excitation flashes (532 nm, 6 ns, 12 mJ) were provided by a Minilite II Nd:YAG laser (Continuum). Measuring light was from a 250-W incandescent tungsten lamp combined with a McPherson monochromator (model 272). Absorption changes were detected with a Hamamatsu Photonics photomultiplier tube (model R928), protected from excitation laser flashes by a second monochromator of the same type. Signals were amplified by a low noise current amplifier (model SR445A; Stanford Research Systems) and digitized with a GaGe Octopus digitizer board (model CS8327, DynamicSignals LLC), maximum sampling rate 50 MHz. Logarithmic filtration of the data was performed using the GageCon program (Waschuk et al., 2005).

Mice. All procedures to maintain and use mice were approved by the Institutional Animal Care and Use Committee at Baylor College of Medicine. ICR (CD-1) female mice were purchased from Baylor College of Medicine Center for Comparative Medicine. C57BL6/J male mice were obtained from Jackson Laboratory (stock numbers 000664). Mice were maintained on a 14 hr:10 hr light:dark cycle with regular mouse chow and water ad libitum. The temperature was maintained at 21-25° and humidity at 40-60%. Experiments were performed during the light cycle. Both male and female mice were used in the experiments at the age of 3-4 weeks.

In utero electroporation. Female ICR mice were crossed with male C57BL6/J mice to obtain timed pregnancies. In utero electroporation was used to deliver the transgenes (Xue et al., 2014). To express HcKCR1 or HcKCR2 in the layer 2/3 pyramidal neurons of the somatosensory cortex, pAAV-CAG-HcKCR1-EYFP or pAAV-CAG-HcKCR2-EYFP (2.5 μg/μl as final concentration) was mixed with pCAG-tdTomato (0.1 μg/μl as final concentration) and Fast Green (Sigma-Aldrich, 0.01% final concentration) for injection. On embryonic day 14.5-15, female mice were anesthetized and a beveled glass micropipette (tip size 100-μm outer diameter, 50-μm inner diameter) was used to penetrate the uterus and the embryo skull to inject about 1.5 μl of DNA solution into one lateral ventricle. Five pulses of current (voltage 39 V, duration 50 ms) were delivered at 1 Hz with a Tweezertrode (5-mm diameter) and a square-wave pulse generator (Gemini X2, BTX Harvard Bioscience). The electrode paddles were positioned in parallel with the brain's sagittal plane. The cathode contacted the same lateral side of the brain of the injected ventricle to target the somatosensory cortex. Transfected pups were identified by the transcranial fluorescence of tdTomato with the MZ10F stereomicroscope (Leica) 1 day after birth.

Brain slice electrophysiology. Mice were used at the age of 3-4 weeks for acute brain slice electrophysiology experiments. Mice were anesthetized by an intraperitoneal injection of a ketamine and xylazine mix (80 mg/kg and 16 mg/kg, respectively) and transcardially perfused with cold (0-4° C.) slice cutting solution containing 80 mM NaCl, 2.5 mM KCl, 1.3 mM NaH₂PO₄, 26 mM NaHCO₃, 4 mM MgCl₂, 0.5 mM CaCl₂, 20 mM D-glucose, 75 mM sucrose and 0.5 mM sodium ascorbate (315 mOsm/L, pH 7.4, saturated with 95% O₂/5% CO₂). Brains were removed and sectioned in the cutting solution with a VT1200S vibratome (Leica) to obtain 300 μm coronal slices. Slices were incubated in a custom-made interface holding chamber containing slice cutting solution saturated with 95% O₂/5% CO₂ at 34° C. for 30 min and then at room temperature for 20 min to 10 hr until they were transferred to the recording chamber.

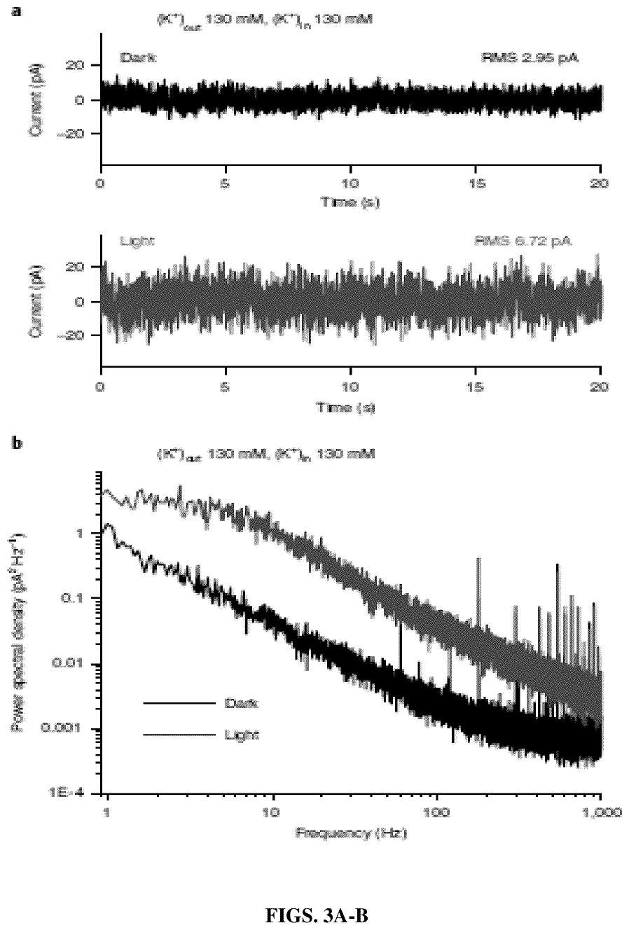

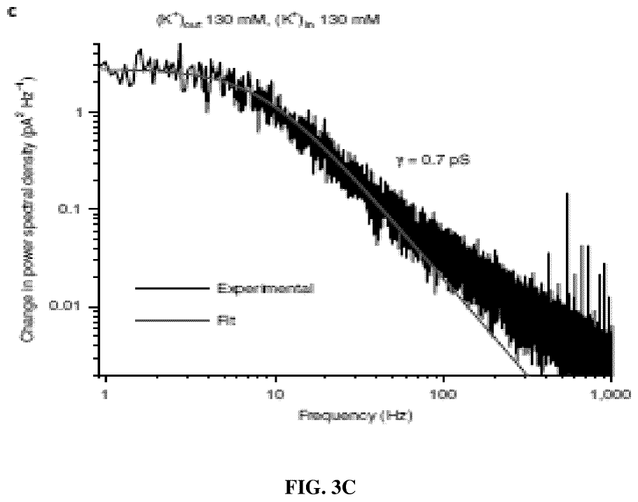

Recordings were performed on submerged slices in artificial cerebrospinal fluid (ACSF) containing 119 mM NaCl, 2.5 mM KCl, 1.3 mM NaH₂PO₄, 26 mM NaHCO₃, 1.3 mM MgCl₂, 2.5 mM CaCl₂), 20 mM D-glucose and 0.5 mM sodium ascorbate (305 mOsm/L, pH 7.4, saturated with 95% O₂/5% CO₂, perfused at 3 ml/min) at 30-32° C. For whole-cell recordings, a K+-based pipette solution containing 142 mM K+-gluconate, 10 mM HEPES, 1 mM EGTA, 2.5 mM MgCl₂, 4 mM ATP-Mg, 0.3 mM GTP-Na, 10 mM Na2-phosphocreatine (295 mOsm/L, pH 7.35) was used. Membrane potentials reported in FIGS. 3a-c were not corrected for liquid junction potential that was experimentally measured as 12.5 mV.

Neurons were visualized with video-assisted infrared differential interference contrast imaging, and fluorescent neurons were identified by epifluorescence imaging under a water immersion objective (40×, 0.8 numerical aperture) on an upright SliceScope Pro 1000 microscope (Scientifica) with an infrared IR-1000 CCD camera (DAGE-MTI). Data were low-pass filtered at 4 kHz and acquired at 10 kHz with an Axon Multiclamp 700B amplifier and an Axon Digidata 1440A Data Acquisition System under the control of Clampex 10.7 (Molecular Devices). Data were analyzed offline using Clampfit (Molecular Devices). For photostimulation, green light was emitted from a collimated 565 nm LED (light-emitting diode; Thorlabs M565L3) to stimulate HcKCR1-expressing neurons and blue light, from a collimated 470 nm LED (Thorlabs M470L3) to stimulate HcKCR2-expressing neurons. The LEDs were driven by a LED driver (Throlabs LEDD1B) under the control of an Axon Digidata 1440A Data Acquisition System and Clampex 10.7. Light was delivered through the reflected light fluorescence illuminator port and the 40× objective.

Photocurrents were recorded by whole-cell voltage clamp in response to 1-s 565 nm light stimulation (13.1 mW mm⁻²) for HcKCR1 and 470 nm light stimulation (18.0 mW mm-2) for HcKCR2. Only recordings with Ra<20 MΩ were included. To test the recovery of photocurrents in the dark, HcKCR1-expressing neurons were held at −45 mV and stimulated with various inter-trial intervals (ITI). To test current-voltage relationship, photocurrents were recorded at membrane voltages from −85 to −45 mV (for HcKCR1) or −70 to −30 mV (for HcKCR2) with 5-mV steps and 30-s ITI. Action potentials of HcKCR1- or HcKCR2-expressing neurons were evoked by injecting a series of 1.5-s depolarizing current pulses (0.1-0.5 nA) in whole-cell current clamp mode. 1-s 565 nm (13.1 mW/mm 2) or 470 nm (18.0 mW mm-2) light stimulation was applied in the middle of current injections with 30-s ITI for HcKCR1 or HcKCR2, respectively. Light stimulation and control trials were interleaved.

Fluorescent imaging of HcKCR expression in neurons. After electrophysiology recordings, brain slices were fixed overnight in 4% paraformaldehyde in PBS (pH 7.4), cryoprotected with 30% sucrose in PBS, and frozen in optimum cutting-temperature medium until sectioning. A HM 450 Sliding Microtome (Thermo Scientific) was used to further section the slices to obtain 50 μm slices. Images were acquired on an Axio Zoom.V16 Fluorescence Stereo Zoom Microscope (Zeiss) or a Sp8X Confocal Microscope (Leica) and processed using MATLAB2021b (MathWorks) and Imaris v. 9.2 (Oxford Instruments). Images were taken from 14 brain slices of 2 male and 2 female mice at the age of 3-4 weeks.

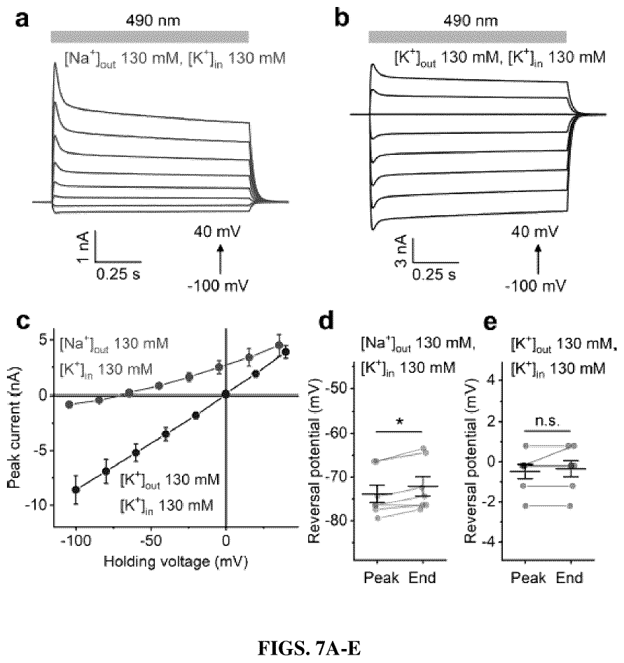

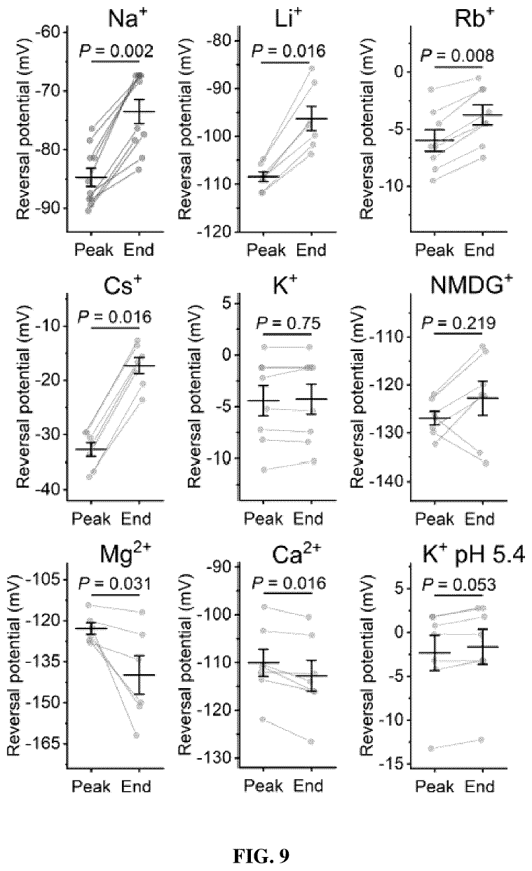

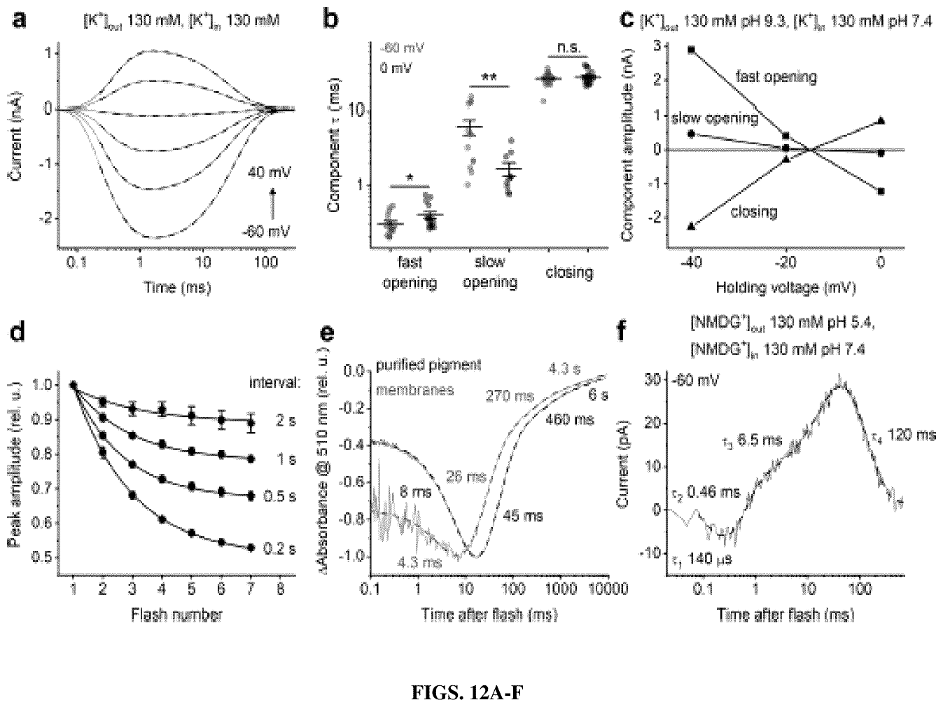

Statistics and reproducibility. In experiments in HEK293/HEK293A cells, identical batches of cell culture were randomly assigned for transfection with each tested construct. Such independently transfected batches were considered independent experiments. At least three separate batches of culture were transfected independently with each construct, their exact number is indicated in figure captions. Photocurrent traces recorded from different cells transfected with the same construct were considered biological replicates (reported as n values). In experiments with 1P excitation with pulses of continuous light and 2P excitation, only one trace per cell/condition was recorded. In experiments under single-turnover conditions (ns laser flashes) used to evaluate channel kinetics by curve fitting, six traces recorded from the same cell were averaged to improve the signal-to-noise ratio. The traces recorded from the same cell were considered technical replicates and never used to derive statistics; statistical analysis was performed on averaged traces (one from each cell) considered as biological replicates. Individual transfected HEK293 cells were selected for patching by inspecting their tag fluorescence; non-fluorescent cells were excluded. Cells in which a GQ seal was not established were automatically discarded from measurements. Photocurrent traces were excluded from the analyses if the Rm decreased to <500 MΩ, or Ra increased to >20 MΩ during recording. In cells that satisfied these criteria, all attempts at replication were successful. Descriptive statistics was calculated by Origin Pro 2016 software. The data are presented as mean±sem values, as indicated in the figure legends; the data from individual cells are also shown when appropriate. No statistical methods were used to pre-determine sample sizes but our sample sizes are similar to those reported in previous publications8,10,34. No normal distribution of the data was assumed; when a specific statistics hypothesis was tested, two-tailed, non-parametric tests [the paired sample signed ranks Wilcoxon test (FIGS. 7d-e and FIG. 9) and the Mann-Whitney test (FIG. 12b)] were used as implemented in Origin software.

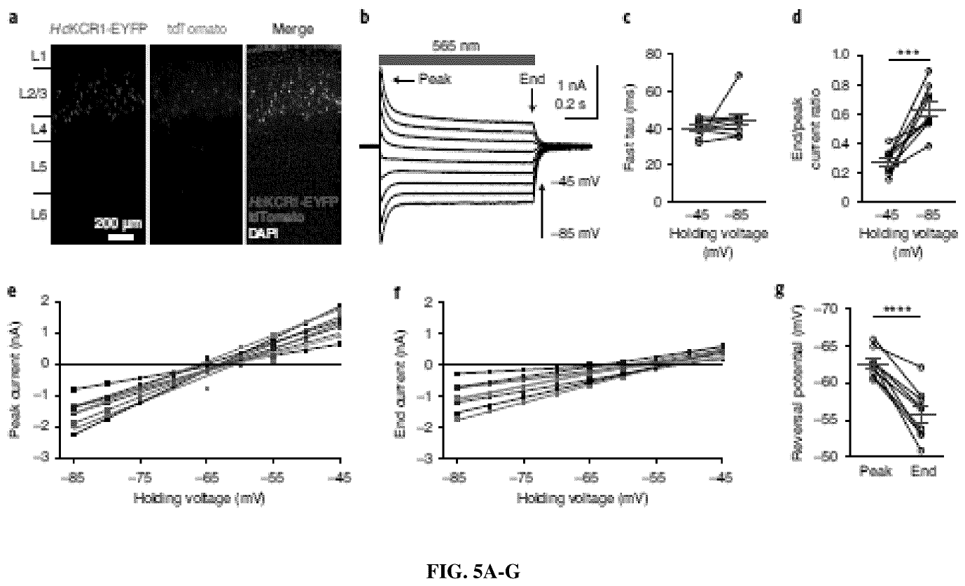

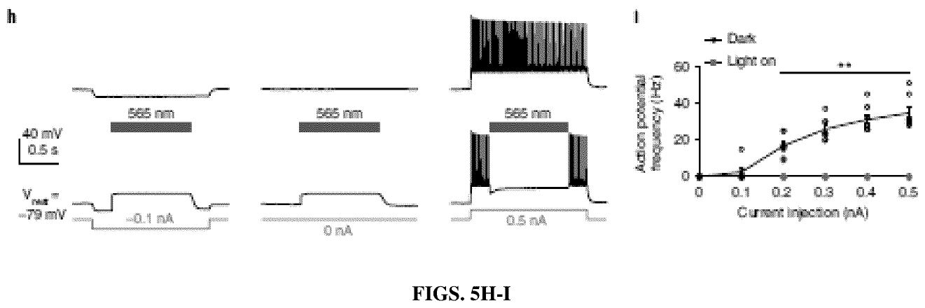

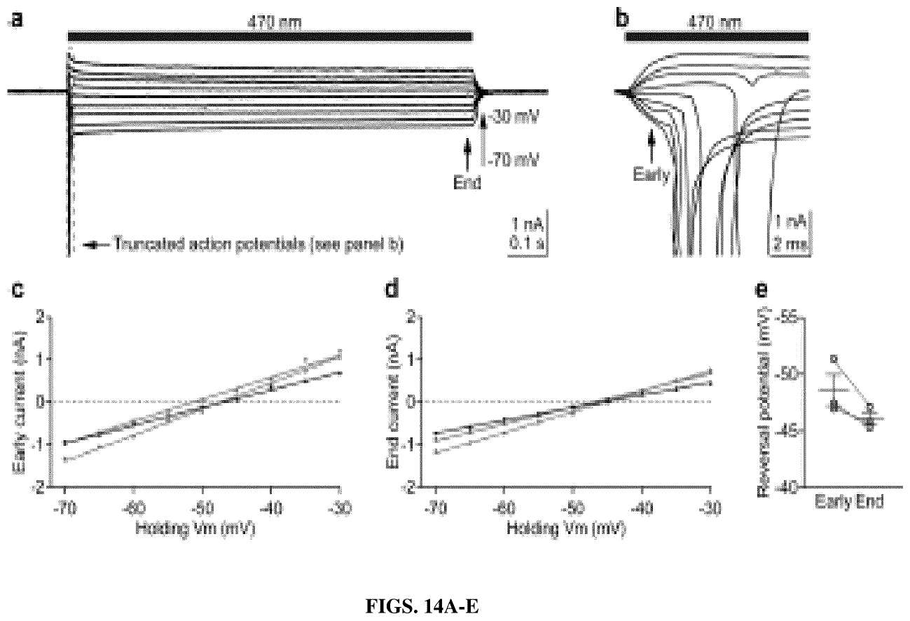

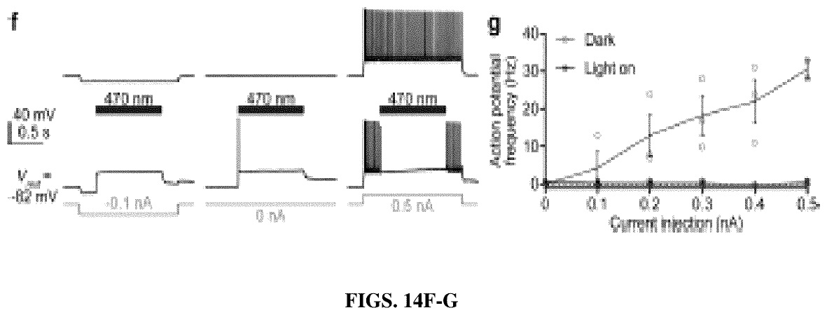

For experiments in neurons, pregnant mice were randomly assigned for in utero electroporation with each tested construct. Statistical analyses were performed with Prism 9 (GraphPad Software). The normality of the data in FIG. 5d, FIG. 5g, FIG. 5i, FIG. 14e, and FIG. 14g were tested by the Anderson-Darling test, D'Agostino & Pearson test, Shapiro-Wilk test, and Kolmogorov-Smirnov test (data in the source files). If the data passed the normality tests, then a statistical test that assumes a Gaussian distribution was used. Otherwise, a statistical test that assumes a non-Gaussian distribution was used. The paired t-test was used in FIG. 5d, FIG. 5g and FIG. 14e, the Wilcoxon matched-pairs signed rank test in FIG. 5c, and the Multiple Wilcoxon matched-pairs signed rank test with multiple corrections in FIG. 5i and FIG. 14g. All statistical tests were two-tailed with an α of 0.05. All reported sample numbers (n) represent biological replicates that are the numbers of recorded neurons.

Data collection and analysis were not performed blind to the conditions of the experiments, because this was an observational study aimed at characterization of KCRs, not their comparison with previously used tools.

Data availability. The file ‘hyphochytrium_catenoides_predicted_proteins_renamed_modified.fasta’ that contains the sequence information of predicted H. catenoides proteins is available from world-wide-web at ebi.ac.uk/biostudies/studies/S-BSST46. The whole genome shotgun sequences FLMG00000000.1 and CAFC00000000.2 are available from world-wide-webe at ncbi.nlm.nih.gov/nuccore/FLMG00000000.1 and world-wide-web at ncbi.nlm.nih.gov/nuccore/CAFC00000000.2, respectively. The sequences of HcKCR1 and HcKCR2 expression constructs are available from GenBank (accession numbers MZ826861 and MZ826862, respectively). The plasmids encoding HcKCR1-mCherry, HcKCR2-mCherry, HcKCR1-EYFP, and HcKCR2-EYFP in a mammalian expression vector backbone are available from Addgene (plasmids 177336, 177337, 182021, and 182022, respectively). Numerical source data are provided with this paper.

Code availability. LogPro, custom software used for logarithmic filtration (noise reduction) of photocurrent traces, is freely available at Zenodo (zenodo.org/record/6461999 #.Y17Zx-jMIuF).

### Example 2—Characterization of KCRs

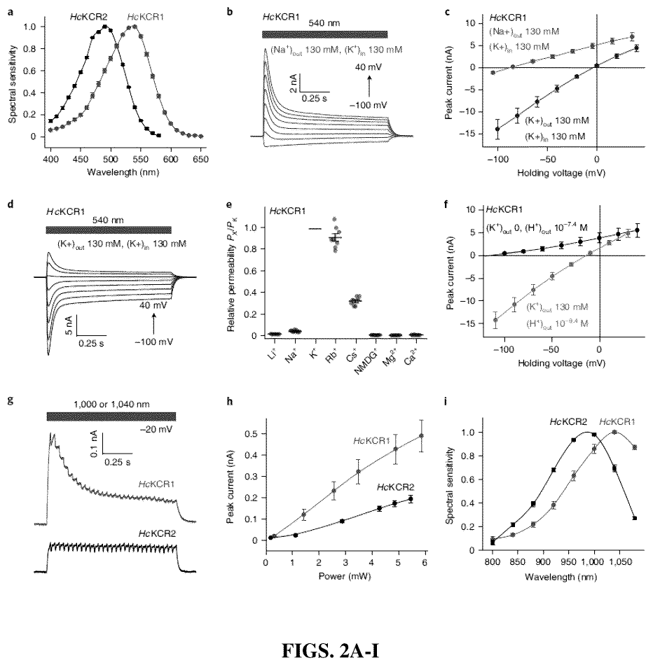

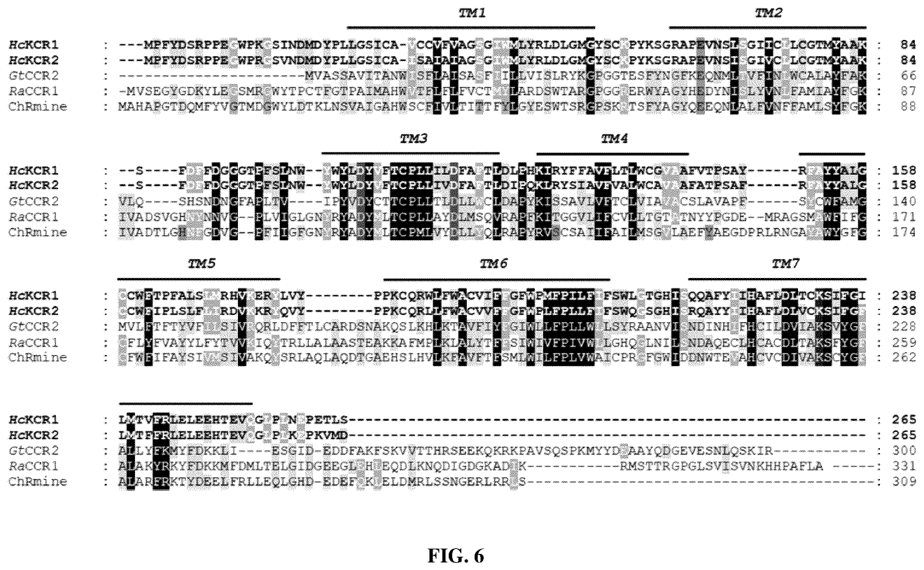

H. catenoides is a heterotrophic organism with a sequenced genome (Leonard, et al., 2018). Two predicted H. catenoides proteins show homology to bacteriorhodopsin-like CCRs (BCCRs) from cryptophyte algae (FIG. 1). In contrast to other known ChRs, BCCRs contain two Asp residues in helix (Wiegert et al., 2017) that are conserved in the H. catenoides homologs (FIG. 6). Recent high-resolution structures of a BCCR known as ChRmine revealed its trimeric organization (Tucker et al., 2021, Kishi et al., 2022), in contrast to other ChRs that form dimers. However, out of the residues implicated in trimer formation in ChRmine, only Glu68 is conserved in the H. catenoides homologs (FIG. 6). Mammalian-codon adapted versions of the polynucleotides encoding the heptahelical transmembrane (rhodopsin) domains were synthesized and expressed them in human embryonic kidney (HEK293) cells as C-terminal mCherry fusions. Both were photoactive; the action spectra of their photocurrents are shown in FIG. 2a. The number 1 to the more red-shifted paralog (spectral maximum 540 nm) and the number 2 to the blue-absorbing pigment (spectral maximum 490 nm).

Photocurrents in response to continuous light. A series of photocurrents generated by HcKCR1 in response to 1-s illumination under physiological ionic conditions (Supplementary Table 1) is shown in FIG. 2b. The voltage dependence of the peak current (IV curve) showed a slight inward rectification and a reversal potential (Vrev) of −85±2 mV (mean±sem, n=10 cells) (FIG. 2c, red). Such behavior has not been observed in any previously tested ChRs and could only be explained by selectivity for K+ over Na+, as the concentration of Cl— was nearly identical on the two sides of the membrane. This conclusion was confirmed by the shift of the Vrev to −3±1 mV (mean±sem, n=7 cells) measured when Na+ in the bath was replaced with K+(FIG. 2c and FIG. 2d, blue). The PK/Pna permeability ratio of HcKCR1 calculated using the modified Goldman-Hodgkin-Katz (GHK) voltage equation (Hille, 2001) was 23, 60 times greater than that of CrChR2. Similar results were obtained with HcKCR2 (FIGS. 7a-e), except that the PK/Pna value of HcKCR2 was 17.

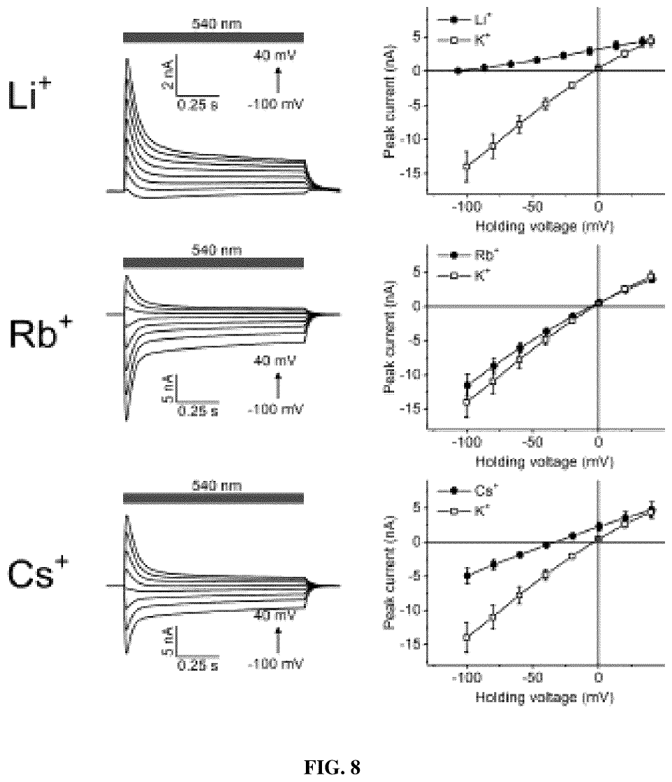

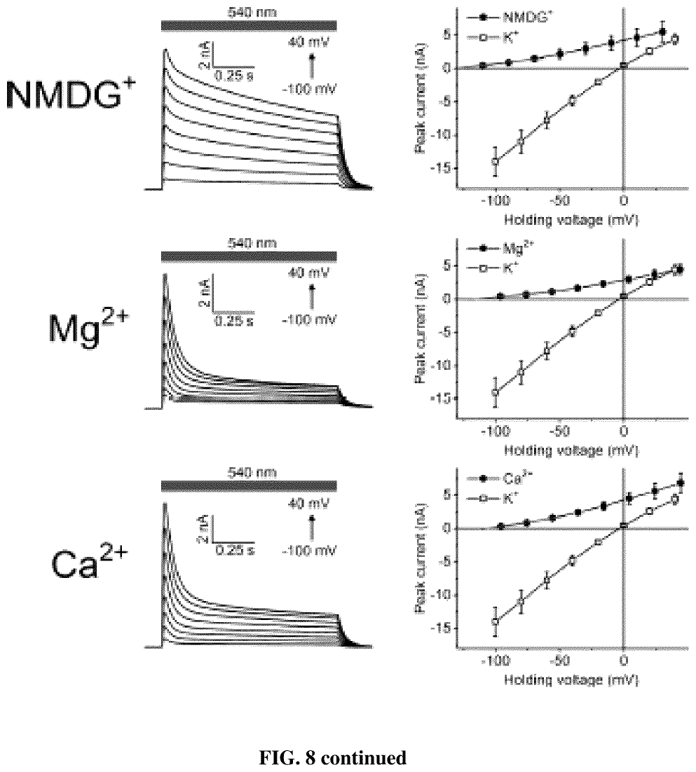

As HcKCR1 exhibited a more red-shifted spectrum, larger current amplitude, and higher selectivity for K+ than HcKCR2, this channel was chosen for a more detailed characterization. Using a similar procedure as described above for Na+, the PX/PK ratios for other metal cations and N-methyl-D-glucamine (NMDG+) were determined. Representative series of photocurrent traces and mean IV curves are shown in FIG. 8, and the PX/PK values, in FIG. 2e. The permeability sequence of HcKCR1 was K+>Rb+>Cs+>Na+>Li+>NMDG+≅Mg2+≅Ca²⁺, as of most voltage- and ligand-gated K+ channels (Eisenman & Horn, 1983). The upper limit of the PH/PK ratio calculated from the experiment shown in FIG. 1f was ˜3×10⁴, which is ˜80 times lower than that of CrChR (Ernst et al., 2014; Richards & Dempski, 2012).

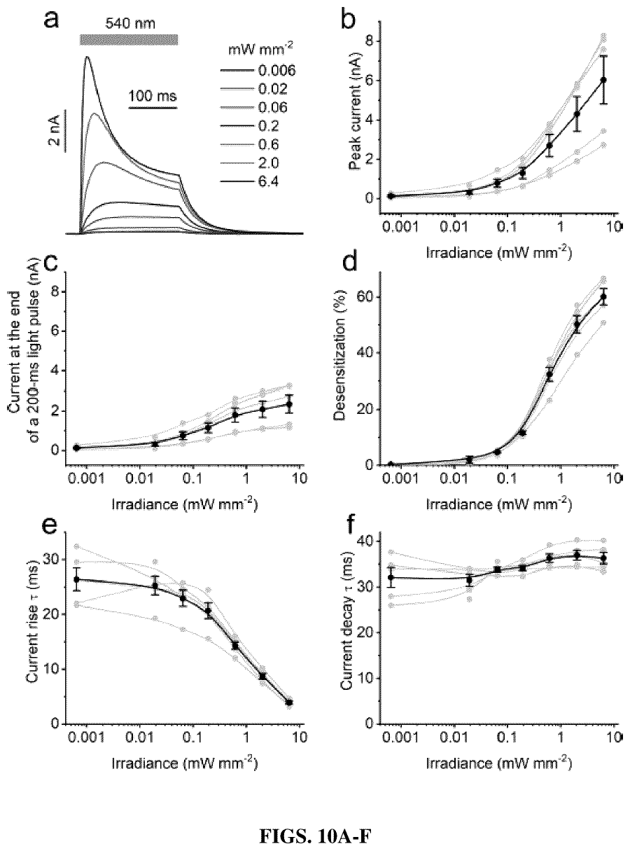

HcKCR1 and HcKCR2 photocurrents decreased to a lower stationary level during illumination (a phenomenon known as desensitization; FIG. 2b and FIG. 7a, respectively). At 0 mV in the Na+ bath, desensitization of HcKCR2 after 1-s illumination (490 nm, 7.9 mW mm-2) was only 32±3% (n=7 cells), whereas that of HcKCR1 (540 nm, 6.4 mW mm-2) was 77±2% (n=9 cells). In the Na+ bath, the Vrev of the desensitized HcKCR1 photocurrent was shifted ˜10 mV to more positive values from that of the peak current (FIGS. 10a-f). Positive Vrev shifts during illumination were also observed with Li+, Rb+ or Cs+ in the bath, but not with other tested cations (FIG. 9). FIG. 10a shows a series of HcKCR1 traces recorded at different stimulus intensities. Desensitized currents exhibited an earlier light saturation than the peak currents (FIG. 10b and FIG. 10c, respectively), as observed in other ChRs9. FIGS. 10d-f shows the dependence of photocurrent desensitization, rise and decay on the light intensity.

ChRs are not suitable for single-channel recording, but their unitary conductance can be estimated by stationary noise analysis (Govorunova et al., 2015; Feldbauer et al., 2009). The noise recorded from HcKCR1-transfected cells increased under continuous illumination, compared to dark conditions (FIG. 3a). The power density spectra for the light and dark noise are shown in FIG. 3b. The unitary conductance (γ), derived from fitting of a Lorentzian function to the light-minus-dark difference spectrum (FIG. 3c), was ˜0.7 pS.

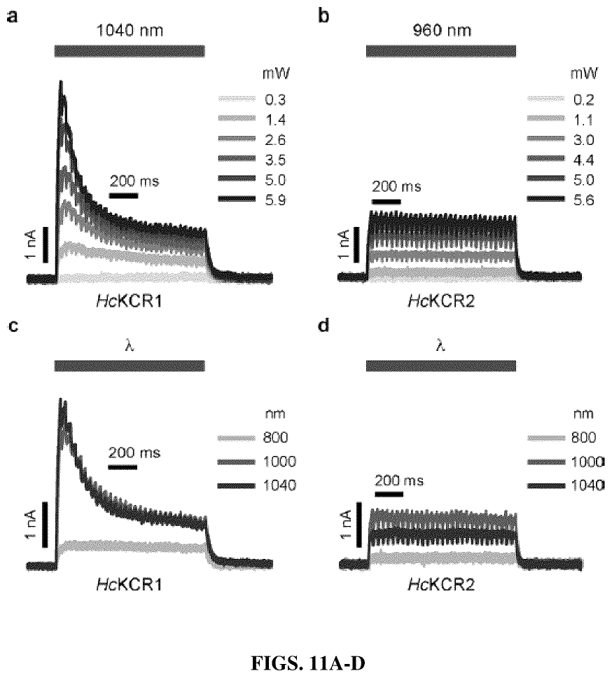

Some rhodopsins absorbing visible light can be excited by infrared (IR) illumination owing to absorption of the second quantum by a virtual energy state generated by absorption of the first photon. After non-radiative deexcitation the molecule returns to the same excited state independently of the wavelength of the absorbed photon(s). Therefore, a high-frequency pulsed IR stimulation is equivalent to stimulation with continuous visible light. Raster scanning was conducted with a conventional two-photon (2P) laser to determine whether HcKCRs can be activated by 2P illumination. As expected, 2P-evoked photocurrent traces (FIG. 2g and FIGS. 11a-b) were similar to those recorded under continuous one-photon (1P) illumination in the visible range (FIG. 2b and FIG. 7a). At the maximal power tested, the peak current amplitude did not saturate (FIG. 2h). The laser wavelength was varied between 800 and 1,080 nm while keeping the power constant (FIGS. 11c-d). The 2P action spectra obtained by measuring the slope of the current rise exhibited the maxima at ˜1,040 and ˜990 nm for HcKCR1 and HcKCR2, respectively (FIG. 2i).

Characterization of HcKCR1 under single-turnover conditions. Photocurrent kinetics measured under continuous illumination reflects the time course of ChR molecules photoactivated at different times during illumination, rather than simultaneously photoactivated ChRs needed to measure the kinetics of channel gating. Furthermore, during continued illumination, photoactive intermediates of the photocycle may absorb a second photon and initiate their own photocycle, the intermediates of which may have different ion selectivity than those of the primary photocycle. Also, the fluence rate of continuous light is insufficient to resolve fast charge displacements within ChR molecules, which can be monitored by a single-turnover laser flash (Sineshchekov et al., 2013).

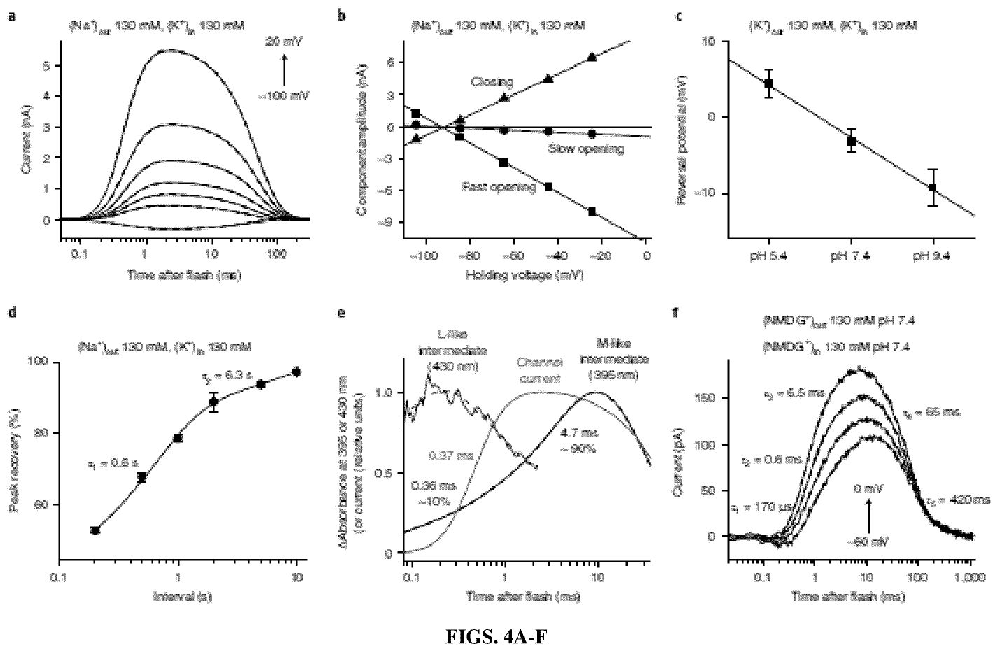

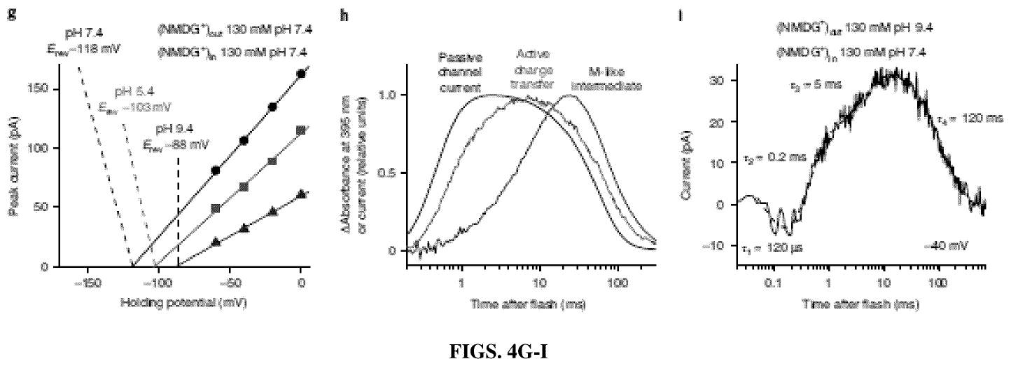

Accordingly, a 6-ns laser flash excitation was used, thereby avoiding second-photon photoproducts, to follow the kinetics of channel gating by monitoring passive channel flux and probe for active (i.e., driven by the energy of the absorbed photon rather than the electrochemical gradient) charge movements within the protein. Regardless of ionic gradients, channel currents could be fit with three exponentials (FIG. 4a and FIG. 12a). Channel opening was biphasic, as in GtACR123 and CrChR224, but channel closing monophasic. The fast opening accelerated, and the slow opening slowed upon depolarization (FIG. 12b). The Vrev of the amplitudes of the three kinetics components were the same in all experimental conditions (FIG. 4b and FIG. 12c), indicating that the relative permeability does not change during the channel cycle. Under symmetrical ionic conditions (130 mM KCl in the bath and pipette) the Vrev only weakly depended on bath pH (FIG. 4c), which confirmed the low permeability of HcKCR1 for protons detected under continuous illumination (FIG. 20. The photocurrent recovery was biphasic with τ=0.6 and 6.6 s (FIG. 4d and FIG. 12d).

Photoactivated rhodopsins undergo a cycle of chemical conversions through a series of spectrally distinct intermediates (Ernst et al., 2014; Kandori, 2020) usually named K, L, M and N/O, following the nomenclature initiated by research in bacteriorhodopsin (Lanyi, 2006). Identification of these intermediates and their kinetics is essential for elucidation of molecular mechanisms of ChR function. For analysis of photochemical conversions, HcKCR1 was expressed in Pichia and purified it in non-denaturing detergent. In both detergent-purified pigment and Pichia membranes multiphasic recovery of the initial unphotolyzed state (FIG. 12e) was observed with τ values similar to those determined electrophysiologically (FIG. 4d). Absorbance at 430 nm dropped concomitant with an increase at 395 nm, characteristic of a transition from an L-like to an M-like intermediate (FIG. 4e). The large blue-shift defining the L to M transition results from proton transfer from the protonated retinylidene Schiff base to the proton acceptor (Kandori, 2020; Lanyi, 2006). Opening of the channel was observed upon transition from the late L to the early M intermediate, which implies the Schiff base deprotonation and channel opening are correlated, but does not necessarily mean a causal relationship. The M rise was biphasic, and τ of the fast component was similar to that of channel opening, unlike GtACR1, in which channel opening takes place in the L state (Sineshchekov et al., 2015; Dreier et al., 2021), and chlorophyte CCRs, in which M rise precedes channel opening (Sineshchekov et al., 2013; Verhoefen et al., 2010).

In addition to passive ion conductance, photoactivation of ChRs results in active intramolecular charge displacements, most notably charge movement caused directly by photoisomerization of all-trans to 13-cis retinal and proton transfers between the retinylidene Schiff base and carboxylates or water molecules in its vicinity (Sineshchekov et al., 2013). Both the acceptor and the donor of the Schiff base proton in the proton pump bacteriorhodopsin are conserved in KCRs, as in cryptophyte BCCRs (FIG. 6). In previously characterized BCCRs, channel activity is tightly coupled to vectorial active translocation of protons within the molecule (Sineshchekov et al., 2017). The inventors probed for intramolecular charge displacements in HcKCR1 by recording photocurrents in the absence of permeable metal cations (FIG. 4f). The initial unresolved negative component of charge movement is a typical reflection of retinal isomerization (Sineshchekov et al., 2013). The voltage dependence of the positive peak crossed the X axis at large negative values characteristic of active charge movement (FIG. 4g). These values remained close when the extracellular pH was varied over 4 units (i.e., 240 mV), indicating that the photoactive site was barely accessible to protons from outside. The rise of positive photocurrent was biphasic with T values similar to those of the components of M rise, indicating active proton transfer from the Schiff base to an outwardly located acceptor (FIG. 4e and FIG. 4f). Both passive channel current and active charge transfer peaked before maximal M accumulation, and decayed in the time window of M decrease (FIG. 4h). This observation suggests that reprotonation of the Schiff base at least partially takes place from an initially protonated acceptor, and there is no proton pumping across the membrane. At pH 9.4 the rates of both positive rise components only slightly increased (FIG. 4i). The decay of active charge movement could be fit with two exponentials with τ=65 and 420 ms (FIG. 40, or a single one with τ˜120 ms (FIG. 4i and FIG. 12f).

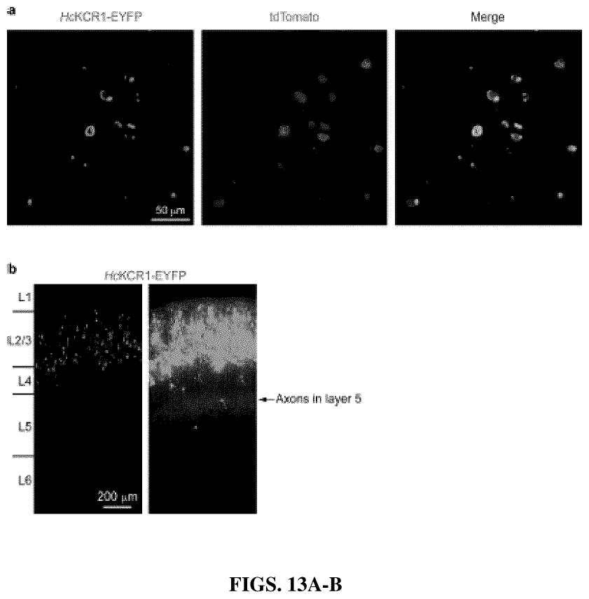

Neuronal inhibition using HcKCRs. Having characterized HcKCRs in HEK293 cells, the inventors next sought to determine the applicability of HcKCRs for optogenetic silencing in neurons. They selectively expressed HcKCR1 or HcKCR2 fused with EYFP in layer 2/3 pyramidal neurons of the mouse somatosensory cortex by in utero electroporation at embryonic day 14.5 or 15 (FIG. 5a). HcKCRs-EYFP were expressed in neurons at high levels and were present in the somata (FIG. 13a), dendrites, and axons (FIG. 13b). Like many native ChRs, they formed some intracellular aggregates (FIG. 13a), indicating incomplete membrane trafficking. Acute brain slices were prepared from 3-4-week-old mice and performed whole-cell voltage clamp recordings from HcKCR1- or HcKCR2-expressing neurons (for solution compositions see Methods). Consistent with its lower PK/Pna value, photoactivation of HcKCR2 sometimes induced an action potential at the onset of illumination (FIGS. 14a-g). Therefore, the inventors focused on HcKCR1 and further characterized its properties in neurons.

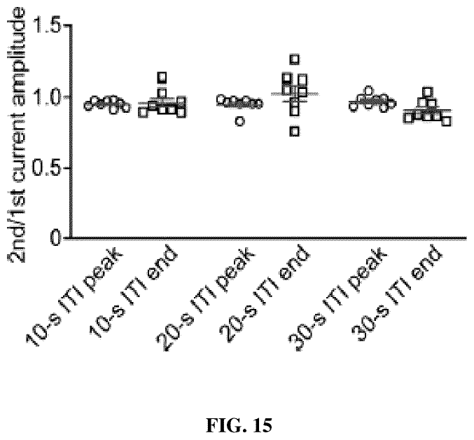

In response to pulses of green light (13.1 mW mm-2), HcKCR1 generated robust photocurrents (FIG. 5b) that recovered quickly in the dark (FIG. 15). The desensitization of photocurrents was best fit by two exponentials with a fast τ of ˜40 ms (FIG. 5c) and a slow τ of 0.6±0.1 and 0.5±0.2 s at −45 and −85 mV, respectively (mean±sem, n=9 neurons from 1 male and 1 female mice, 3-4 weeks old). The ratio of the photocurrent amplitude at the end of 1-s illumination to the peak amplitude increased when the membrane was hyperpolarized (FIG. 5d). The IV curves showed a reversal potential of −63 mV for peak photocurrents and −56 mV for end photocurrents (FIGS. 5e-g), indicating that channel states formed upon absorption of a second photon under continuous light stimulation alter the relative permeability in favor of Na+.

Current clamp recordings were done to study HcKCR1 as a neuronal silencing tool. HcKCR1-expressing neurons showed resting membrane potentials of −77.2±1.3 mV, input resistances of 133.7±11.3 MΩ, and action potential thresholds of −37.7±0.7 mV (mean±sem, n=8 neurons from 1 male and 1 female mice, 3-4 weeks old). When the membrane potential was at rest, photoactivation of HcKCR1 depolarized the membrane to −57.9±1.4 mV (mean±sem) at the end of 1-s illumination (FIG. 5h). When the membrane potential was hyperpolarized to −90.0±1.4 mV (mean±sem) by injecting −0.1 nA current, photoactivation of HcKCR1 depolarized the membrane to a similar value of −58.6±2.1 mV (mean±sem) at the end of illumination (FIG. 5h). These results indicate that the photocurrents are sufficient to bring the membrane potential to the reversal potential of HcKCR1 (FIG. 5f, FIG. 5g). When current injections depolarized the membrane potential to evoke action potentials, photoactivation of HcKCR1 inhibited all action potentials during illumination (FIG. 5h, FIG. 5i), demonstrating that HcKCR1 is a potent optogenetic silencer of mouse cortical neurons.

### Example 3—Discussion

Among currently known ChRs, KCR sequences are most closely related to those of cryptophyte BCCRs (FIG. 1). However, out of 15 so far tested BCCRs functional in heterologous systems, none exhibits K+ selectivity30-32. Therefore, KCRs can be regarded as a separate functional class of ChRs for their unique selectivity properties. The discovery of KCRs reveals an alternative mechanism for K+ selection, as compared to that of the well-characterized voltage- and ligand gated K+ channels, and our analysis of KCR photocurrents under single-turnover conditions lays the basis for its elucidation.

The relationship between Schiff base protonation, active proton transfers, and passive conductance of ChRs is one of the key questions in ChR research. By kinetic comparison of single-turnover photocurrents and transient absorption changes it was shown that opening of the HcKCR1 channel takes place upon transition of the pigment from the late L-like intermediate to the early M-like intermediate (with the deprotonated Schiff base) (FIG. 4e). This is a basic difference of HcKCR1 from both classical CCRs and ACRs. HcKCR1 also differs from these ChRs by a strong isolation of the photoactive site from protons in the external aqueous phase (FIG. 4g).

Another important finding is that the channel's selectivity doesn't change during the single-turnover photocycle (FIG. 4b). This means that the Vrev shift observed under continuous illumination in the Na+ bath (FIG. 5g and FIG. 9) is caused by the absorption of a second quantum by photocycle intermediates. Among physiological cations, only Na+ caused such Vrev shift (FIG. 9), which means that the relative permeability for K+ is reduced in favor of Na+. Previous studies have shown that different long-lived photocycle intermediates accumulate upon prolonged illumination in different ChRs (Kuhne et al., 2019; Sineshchekov et al., 2020; Oppermann et al., 2019). Characterization of the photocycle intermediates and their conductance, vital for engineering of KCRs with better characteristics (e.g., less desensitization), is in progress.

Red light better penetrates biological tissue and therefore red-shifted tools are desirable for optogenetic applications. The HcKCR1 absorption maximum is 540 nm, but ChRs are known with even more red-shifted spectra (590-610 nm), such as Chrimson (Klapoetke et al., 2014) and RubyACRs (Govorunova et al., 2020; Patent Publication Number US20220056100A1). The absorption wavelength in rhodopsins is mostly determined by residues of the retinal-binding pocket. Comparison of the pockets (Supplementary Table 2) suggests residue positions, mutations at which are likely to shift the spectrum of HcKCR1 to longer wavelengths. For example, Gly 158 in HcKCR1, which corresponds to Ser 141 in bacteriorhodopsin, is conserved in Chrimson and RubyACRs. This polar Ser, located near the f3-ionone ring of the retinal chromophore, causes a red spectral shift by electrostatic interaction (Hoffmann et al., 2006).

HcKCR1 expands the optogenetic toolbox with a natural K+-selective tool that benefits from the high efficiency provided by evolution, enabling direct, rapid, and potent photocontrol of K+ transmembrane gradients in mouse cortical neurons (FIGS. 5a-i). Its major advantage is 1,000 times faster current kinetics than that of other optogenetic (Supplementary Table 3) and photopharmacogenetic tools (reviewed in Wiegert et al., 2017) available for manipulation of the K+ gradients. The unitary conductance of HcKCR1 measured under prolonged illumination (i.e. in a desensitized state) is similar to that of GtACRs9 and ˜20 times higher than that of CrChR221. The PK/Pna of HcKCR1 (˜23) is higher than that of the viral potassium channel KCV (˜9) (Plugge et al., 2000), but smaller than that of mammalian voltage-gated K+ channels (100-1,000) (Mironenko et al., 2021). Residual permeability for Na+ shifts the Vrev of HcKCR1 photocurrents to more depolarized values from the Nernst equilibrium potential for K+. This could lead to photoinduced depolarization in some neuronal types and/or activity states, which needs to be tested in future experiments. Mechanistic understanding of HcKCR1 conductance may help engineer this channel for better K+ selectivity to avoid this potential problem. The intracellular aggregation of HcKCRs in neurons is likely to affect cell health, particularly in long-term experiments. Research is in progress to test whether this is the case, and whether additional trafficking motifs will reduce intracellular aggregation and improve membrane targeting of HcKCRs.

2P illumination with IR light is the method of choice for optogenetic stimulation or inhibition with single-cell resolution in three-dimensional tissues in vivo39,40. Our experiments show that both KCRs can be activated using 2P illumination (FIG. 2g-I and FIGS. 11a-d, paving the way for their deployment for in vivo optogenetic silencing experiments targeting individual cells or neuronal processes41. The 2P spectra of both KCRs are well matched to the excitation wavelengths of common (Ti:Sapphire) lasers used in neuroscience labs, which can typically be tuned from ˜700 nm to ˜1,020-1,300 nm. The most powerful commercially available 2P lasers, which excite at ˜1,040 nm, enable simultaneous 2P illumination of large ensembles of single neurons in rodents40. Given its peak excitation at ˜1,040 nm, HcKCR1 is expected to be ideally suited for such applications. As 2P photocurrents did not saturate at the highest tested laser power (FIG. 2h), larger currents should be achievable. Alternatively, more efficient illumination methods such as scanless 2P excitation (Papagiakoumou et al., 2010) can be used.

HcKCRs are the first known ChRs highly selective for K+ over Na+. They present an alternative mechanism for K+ selectivity, compared with voltage- and ligand-gated K+ channels. Owing to its higher K+ selectivity, larger photocurrents, and more red-shifted absorption, HcKCR1 is better suited as a tool for optogenetic neuronal silencing than HcKCR2. HcKCRs will be useful for manipulation of K+ gradients in various cell types with high spatiotemporal precision, and for the study and treatment of K+ channelopathies.

Supplementary Table 1. Composition of pipette and bath solutions and liquid junction
potentials in experiments with HEK293/HEK293A cells.

| | | | | | | | | | | | LJP |
|---|---|---|---|---|---|---|---|---|---|---|---|
| | | | | | | | | | | | pip. |
| | NaCl | KCl | LiCl | RbCl | CsCl | NMDG | MgCl₂ | CaCl₂ | HEPES | Glucose | stand |
| Pipette | — | 130 | — | — | — | — | 2 | — | 10 | — | — |
| standard | | | | | | | | | | | |
| Pipette | — | — | — | — | — | 130 | 2 | — | 10 | — | — |
| NMDG⁺ | | | | | | | | | | | |
| Bath Na⁺ | 130 | — | — | — | — | — | 2 | 2 | 10 | 10 | 4.4 |
| Bath K⁺ | — | 130 | — | — | — | — | 2 | 2 | 10 | 10 | 0.2 |
| Bath Li⁺ | — | — | 130 | — | — | — | 2 | 2 | 10 | 10 | 6.7 |
| Bath Rb⁺ | — | — | — | 130 | — | — | 2 | 2 | 10 | 10 | −0.5 |
| Bath Cs⁺ | — | — | — | — | 130 | — | 2 | 2 | 10 | 10 | −0.4 |
| Bath | — | — | — | — | — | 130 | 2 | 2 | 10 | 10 | 9.9 |
| NMDG⁺ | | | | | | | | | | | |
| Bath Mg²⁺ | — | — | — | — | — | — | 65 | 2 | 10 | 10 | −3.8 |
| Bath Ca²⁺ | — | — | — | — | — | — | 2 | 65 | 10 | 10 | −4.2 |

Abbreviations:
HEPES, 4-(2-hydroxyethyl)-1-piperazineethanesulfonic acid;
LJP, liquid junction potential;
NMDG, N-Methyl-D-glueamine.
All concentrations are in mM.

Supplementary Table 2. Comparison of amino acid residues forming the retinal-binding
pockets of HcKCR1 and red-shifted ChRs.

| | 49 | 53 | 57 | 85 | 89 | 90 | 93 | 115 | 138 | 141 | 142 | 145 | 182 | 185 | 188 | 189 |
|---|---|---|---|---|---|---|---|---|---|---|---|---|---|---|---|---|
| HcKCR1 | L | I | C | D | T | C | L | L | Y | G | C | F | W | F | P | F |
| Chrimson | V | E | V | E | S | C | I | C | Y | S | C | G | W | Y | P | W |
| RubyACRs | V | T | Y | F | T | C | I | D | N | S | Y | C | Q | F | I | Y |
| CsCCR | I | E | Y | G | T | T | I | N | F | G | C | M | W | F | P | W |
| BR | V | A | Y | D | T | T | L | D | W | S | T | M | W | F | P | W |

Abbreviations:
CsCCR, Cristomastix stigmatica cation channelrhodopsin;
BR, bacteriorhodopsin.
The numbers are the residue positions in bacteriorhodopsin.

Supplementary Table 3. Comparison of HcKCR1 with previously available tools for
optogenetic manipulation of K⁺ gradients.

| | | | | Half- | | | |
|---|---|---|---|---|---|---|---|
| | | | | saturation | | | |
| | | | | light | | | |
| | | | | intensity | | | |
| | | | P_{K}/ | (mW | Rise τ | Decay τ | |
| Name | Modality | Activation | P_{Na} | mm⁻²) | (ms) | (ms) | Reference |
| HcKCR1_mCherry | natural | Direct | 23 | 0.2* | 3.6 | 36 | This |
| | channelrhodopsin | | | | | | study |
| BLINK2 | viral K⁺ channel | Direct | 9.3^{#} | n.d. | 162,000 | 444,000 | 13, 38 |
| | K_{CV} fused with | | | | | | |
| | LOV2 domain | | | | | | |
| SthK-T-YFP-bPAC- | Spirochaeta K⁺ | Indirect | n.d. | 0.5 | 4,000 | 254,000 | 14 |
| Ex | channel fused | | | | | | |
| | with Beggiatoa | | | | | | |
| | photoactivated | | | | | | |
| | adenylyl cyclase | | | | | | |
| SthK-P2A- | Spirochaeta K⁺ | Indirect | n.d. | 0.85 | 12,000 | 33,000 | 15 |
| bPAC_mCherry | channel | | | | | | |
| | coexpressed | | | | | | |
| | with Beggiatoa | | | | | | |
| | photoactivated | | | | | | |
| | adenylyl cyclase | | | | | | |

*For current at the end of a 200-ms light pulse;
^{#}determined in natural K_{CV};
n.d., not determined.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IV. REFERENCES

The following references, to the extent that they provide exemplary procedural or other details supplementary to those set forth herein, are specifically incorporated herein by reference.
Alberio et al., Nat Methods 15, 969-976, (2018).
Beck et al., Front. Neurosci. 12, 643, (2018).
Bernal Sierra et al., Nat. Commun. 9, 4611, (2018).
Deisseroth, Nat. Methods 8, 26-29, (2011).
MacKinnon, FEBS Lett. 555, 62-65, (2003).
Dreier et al., Commun Biol 4, 578, (2021).
Eisenman & Horn, J. Membr. Biol. 76, 197-225, (1983).
Ernst et al., Chem. Rev. 114, 126-163, (2014).
Feldbauer et al., Proc. Natl. Acad. Sci. USA 106, 12317-12322, (2009).
Govorunova et al., Science 349, 647-650, (2015).
Govorunova et al., Biophys. J. 110, 2302-2304, (2016).
Govorunova et al., Proc. Natl. Acad. Sci. USA 117, 22833-22840, (2020).
Govorunova et al., MBio 12, e0165621, (2021).
Hille, Sinauer Associates, (2001).
Hoffmann, et al., J. Am. Chem. Soc. 128, 10808-10818, (2006).
Kandori, Biophys. Rev. 12, 355-361, (2020).
Klapoetke et al., Nat. Methods 11, 338-346, (2014).
Kishi, et al. Cell 185, 672-689.e623, (2022).
Krogh et al., J. Mol. Biol. 305, 567-580, (2001).
Kuhne et al., Proc. Natl. Acad. Sci. USA 116, 9380-9389, (2019).
Lanyi, Biochim. Biophys. Acta 1757, 1012-1018, (2006).
Leonard et al., Open Biol. 8, 170184, (2018).
Mironenko et al., J. Mol. Biol. 433, 167002, (2021).
Oppermann et al., Nat. Commun. 10, 3315, (2019).
Papagiakoumou et al., Nat. Methods 7, 848-854, (2010).
Plugge et al., Science 287, 1641-1644, (2000).
Richards & Dempski, PLoS One 7, e50018, (2012).
Sineshchekov et al., Proc. Natl. Acad. Sci. USA 114, E9512-E9519, (2017).
Sineshchekov et al., Biophys. J. 104, 807-817, (2013).
Sineshchekov et al., Proc. Natl. Acad. Sci. USA 112, 14236-14241, (2015).
Sineshchekov et al., mBio 11, e00657-00620, (2020).
Tucker et al., BioRxiv, (2021).
Wiegert et al., Neuron 95, 504-529, (2017).
Waschuk et al., Proc. Natl. Acad. Sci. USA 102, 6879-6883, (2005).
Xue et al., Nature 511, 596-600, (2014).
Verhoefen et al., Chemphyschem 11, 3113-3122, (2010).

1. A recombinant nucleic acid operatively linked to a heterologous promoter sequence, said recombinant nucleic acid comprising:
(a) a kalium-conducting rhodopsin domain encoded by a nucleic acid sequence that encodes a polypeptide sequence that is at least about 90% identical to SEQ ID NO: 1 or SEQ ID NO: 2; and/or
(b) a kalium-conducting rhodopsin domain encoded by a nucleic acid sequence that is at least about 90% identical to a sequence selected from the group consisting of SEQ ID NO: 3, SEQ ID NO: 4, SEQ ID NO: 5 and SEQ ID NO: 6.

2. (canceled)

3. An expression vector comprising the recombinant nucleic acid of claim 1.

4. A recombinant host cell comprising a recombinant nucleic acid of claim 1.

5. The recombinant host cell of claim 4, wherein said host cell is an isolated human cell.

6. The recombinant host cell of claim 4, wherein said host cell is a non-human mammalian cell.

7. The recombinant host cell of claim 4, wherein said host cell is a bacterial cell or a yeast cell.

8.-11. (canceled)

12. A method of membrane hyperpolarization of a cell in a subject suffering from a neuron mediated disorder, said method comprising delivering to the said cell of said subject an expression vector comprising:
(a) a polynucleotide sequence that encodes a kalium-conducting rhodopsin domain that is at least about 90% identical to SEQ ID NO: 1 or SEQ ID NO: 2; and/or
(b) a polynucleotide sequence at least about 90% identical to a sequence selected from the group consisting of SEQ ID NO: 3, SEQ ID NO: 4, SEQ ID NO: 5, and SEQ ID NO: 6, encoding a kalium-conducting rhodopsin domain expressible in said cell.

13. The method of claim 12, wherein the said expressed rhodopsin domain silences a signal from said cell.

14. The method of claim 12, wherein said cell is a target neuron, wherein said expressed rhodopsin results in silencing of a signal from said target neuron.

15. The method of claim 12, wherein said cell is a retinal neuron, wherein the said expressed rhodopsin produces a high level of membrane potential in said retinal neuron.

16. The method of claim 12, wherein, said polynucleotide sequence is operatively linked to a promoter sequence.

17-18. (canceled)

19. The method of claim 12, wherein said subject is mammalian.

20. The method of claim 12, wherein said subject is human.

21. The method of claim 12, wherein said delivering comprises a pharmaceutically acceptable carrying agent.

22-26. (canceled)

27. An isolated nucleic acid molecule comprising:
(a) a polynucleotide sequence that encodes a kalium-conducting rhodopsin domain that is at least about 90% identical to SEQ ID NO: 1 or SEQ ID NO: 2; and/or
(b) a polynucleotide sequence encoding a kalium-conducting channelrhodopsin domain having a sequence at least about 90% identical to a sequence selected from the group consisting of SEQ ID NO: 3, SEQ ID NO: 4, SEQ ID NO: 5, and SEQ ID NO: 6.

28. (canceled)

29. The isolated nucleic acid molecule of claim 27, where said nucleic acid is a DNA.

30. The isolated nucleic acid molecule of claim 27, where said nucleic acid is an RNA.

31. The isolated nucleic acid molecule of claim 30, where said nucleic acid is an mRNA.

32. An expression vector comprising a nucleic acid molecule according to claim 27.

33-52. (canceled)

53. The vector of claim 32, wherein the vector is a plasmid, a viral vector, or an episomal vector.

54-57. (canceled)
